(19) United States
(12) Patent Application Publication
(10) Pub. No.: US 2022/0332776 A1
(43) Pub. Date: Oct. 20, 2022

(54) ANTIBODY CHEMICALLY INDUCED DIMERIZERS (AbCID) AS MOLECULAR SWITCHES AND USES THEREOF

(71) Applicant: The Regents of the University of California, Oakland, CA (US)

(72) Inventors: James A. WELLS, Oakland, CA (US); Zachary B. HILL, Oakland, CA (US); Alexander J. MARTINKO, Oakland, CA (US)

(21) Appl. No.: 17/633,743

(22) PCT Filed: Aug. 6, 2020
(86) PCT No.: PCT/US2020/045160

§ 371 Date: Feb. 8, 2022

Related U.S. Application Data
(60) Provisional application No. 62/885,164, filed on Aug. 9, 2019.

(51) Int. Cl. C07K 14/47; C12N 9/22; C07K 16/28; A61K 38/46; A61K 38/17; A61K 39/395; A61K 35/17; A61P 37/02; C07K 16/44; A61K 31/454; A61K 31/711

(52) U.S. Cl. CPC C07K 14/4702; C12N 9/22; C07K 16/2809; C07K 16/2803; A61K 38/465; A61K 38/1709; A61K 39/3955; A61K 35/17; A61P 37/02; C07K 16/44; A61K 31/454; A61K 31/711; C07K 2319/03; C07K 2319/02; C07K 2319/33; A61K 2039/5158

(57) ABSTRACT

The present invention provides antibody-based chemically induced dimerizers (AbCIDs) comprising an antibody domain that recognizes a chemical epitope created by the binding of an IMiD to a binding partner, such as cereblon, and methods to rapidly generate such AbCIDs. Several aspects described herein relate to systems, compositions, and methods including components of a chemically induced dimerizer (CID), such as an antibody chemically induced dimerizer (AbCID).

### CROSS-REFERENCE TO RELATED PATENT APPLICATIONS

The present patent application claims benefit of priority to U.S. Provisional Patent Application No. 62/885,164, filed Aug. 9, 2019. The disclosure of the above-referenced application is herein expressly incorporated by reference in its entirety, including any drawings.

### STATEMENT AS TO RIGHTS TO INVENTIONS MADE UNDER FEDERALLY SPONSORED RESEARCH OR DEVELOPMENT

This invention was made with government support under grant nos. R01 CA191018 and R01 GM097316 awarded by the National Institutes of Health. The government has certain rights in the invention.

### FIELD

The present disclosure relates to synthetic antibody chemically induced dimerizer (AbCID) systems that function as molecular switches. Further provided are compositions comprising system elements and methods of using the systems, such as for the treatment of various diseases and conditions.

### INCORPORATION OF THE SEQUENCE LISTING

The content of the electronically submitted sequence listing (Name: 048536-650001WO_SL_ST25.txt, Size: 11,965 bytes; and Date of Creation: Aug. 4, 2020) is herein incorporated by reference in its entirety.

### BACKGROUND OF THE INVENTION

Chemically induced dimerizers (CIDs) are powerful tools for dose and temporal control over protein-protein interactions (Spencer, et al., Science, 262:1019-1024 (1993); Clackson, T., Chemical Biology, pgs. 227-249 (2008); Fegan, et al., Chem. Rev., 110:3315-3336 (2010); Putyrski, et al., FEBS Lett, 586:2097-2105 (2012)). CIDs have been applied to the development of artificial cellular circuits (Lienert, et al., Nat. Rev. Mol. Cell Biol., 15:95-107 (2014)), activating split-enzyme activity (Shekhawat, et al., Curr. Opin. Chem. Biol., 15:789-797 (2011); Nguyen, et al., Nat. Commun., 7:12009 (2016); Zetsche, et al., Nat. Biotechnol., 33:139-142 (2015); Pelletier, et al., Proc. Natl. Acad. Sci. USA, 95:12141-12146 (1998)), and more recently used in the clinic as safety switches for next-generation T-cell therapies (Straathof, et al., Blood, 105:4247-4254 (2005); Di Stasi, et al., N Engl. J. Med., 365:1673-1683 (2011)). A number of homo- and hetero-CIDs have been developed but the vast majority are limited to pieces of natural proteins known to bind the small molecule inducers, such as the prototypical rapamycin-FKBP12-FRB system (Spencer, et al., Science, 262:1019-1024 (1993); Ho, et al., Nature, 382:822-826 (1996); Belshaw, et al., Proc. Natl. Acad. Sci. USA, 93:4604-4607 (1996); Rivera, et al., Nat. Med., 2:1028-1032 (1996); Farrar, et al., Nature, 383:178-181 (1996); Miyamoto, et al., Nat. Chem. Biol., 8:465-470 (2012); Erhart, et al., Chem. Biol., 20:549-557 (2013); Kopytek, et al., Chem. Biol., 7:313-321 (2000); Liang, et al., Sci. Signal., 4, rs2 (2011); Czlapinski, et al., J. Am. Chem. Soc., 130:13186-13187 (2008)). Currently, no general method to design or identify these tools exists. However, the expanded use of and interest in CIDs for multiplexed control of biological events in cells and animals necessitates invention of many more small-molecule-inducible systems. Moreover, CIDs have promise in human therapy but there is no systematic means of generating CID based on human derived parts to reduce or eliminate the risk of immunogenicity.

Previous workers have shown it is possible to use phage display to generate antibodies that could specifically bind to protein conformations “trapped” by binding of small molecules (Gao, et al., Proc. Natl. Acad. Sci. USA, 106:3071-3076 (2009); Rizk, et al., Nat. Struct. Mol. Biol., 18:437-442 (2011); Staus, et al., Nature, 535:448-452 (2016); Thomsen, et al., Proc. Natl. Acad. Sci. USA, 110:8477-8482 (2013)). In these cases, the antibody shows an increased affinity for the small-molecule-bound form of the protein, similar to a CID. However, the antibody is often able to bind the protein in the trapped conformation, independent of the small molecule. For this reason, the selectivity of conformation-selective antibodies for the bound form over the apo form is limited, reducing their utility as selective CIDs. Thus, there is a need in the art for improved CIDs. Moreover, the application of CID technology for human therapy to regulate engineered proteins or cells is highly dependent on using human-derived protein scaffolds to reduce immunogenicity. To date the only CID system that contains fully human parts is the FKBP-FRB CID and the small molecules used to activate it are either toxic or lacking in drug-like properties in their own right. Thus, there is a need to expand the development of CIDs with human derived proteins or antibodies.

### SUMMARY

Several aspects described herein relate to systems, compositions, and methods including components of a chemically induced dimerizer (CID), such as an antibody chemically induced dimerizer (AbCID).

In one aspect, provided here is a system comprising: (a) a first chemically induced dimer (CID) component comprising a first binding moiety capable of interacting with an immunomodulatory imide drug (IMiD) selected from thalidomide and analogs thereof to form a complex between the first CID component and the IMiD, or a first nucleic acid encoding polypeptide components of the first CID component; and (b) a second CID component comprising a second binding moiety that specifically binds to the complex between the first CID component and the IMiD, or a second nucleic acid encoding polypeptide components of the second CID component. In some embodiments, the first binding moiety comprises cereblon or an IMiD-binding fragment or derivative thereof. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and one atom of the first binding moiety. In some embodiments, the second binding moiety is an antibody moiety that specifically binds to a chemical-epitope comprising at least a portion of the IMiD and a portion of the first binding moiety. In some embodiments, the second binding moiety is an antibody moiety comprising heavy chain and light chain complementarity determining regions (CDRs) from a Fab-phage clone according to Table 1. In some embodiments, the IMiD is thalidomide, lenalidomide, or pomalidomide. In some embodiments, the first binding domain comprises the amino acid sequence of SEQ ID NO: 25 or a variant thereof having at least 85% identity to the amino acid sequence of SEQ ID NO: 25.

In some embodiments, according to any of the systems described above, the first CID component further comprises a first adapter moiety linked to the first binding moiety and/or the second CID component further comprises a second adapter moiety linked to the second binding moiety.

In some embodiments, according to any of the systems described above where the first CID component further comprises a first adapter moiety and the second CID component further comprises a second adapter moiety, (a) the first adapter moiety comprises a DNA binding domain and the second adapter moiety comprises a transcriptional regulatory domain; or (b) the second adapter moiety comprises a DNA binding domain and the first adapter moiety comprises a transcriptional regulatory domain, wherein the first CID component and the second CID component are configured such that when dimerized in the presence of the IMiD to form the CID, the CID is capable of regulating transcription of a target gene. In some embodiments, (a) the transcriptional regulatory domain is a transcriptional activation domain, and the CID is capable of upregulating transcription of the target gene; or (b) the transcriptional regulatory domain is a transcriptional repressor domain, and the CID is capable of downregulating transcription of the target gene. In some embodiments, the DNA binding domain is derived from a naturally occurring transcriptional regulator. In some embodiments, the DNA binding domain is derived from an RNA-guided endonuclease or a DNA-guided endonuclease. In some embodiments, the RNA-guided endonuclease or DNA-guided endonuclease is catalytically dead. In some embodiments, the DNA binding domain is derived from a catalytically dead Cas9 (dCas9).

In some embodiments, according to any of the systems described above where the first CID component further comprises a first adapter moiety and the second CID component further comprises a second adapter moiety, the first CID component and the second CID component are configured such that when dimerized in the presence of the IMiD to form a CID associated with a target cell, the CID is capable of inducing target cell death. In some embodiments, the first adapter moiety and the second adapter moiety are together capable of inducing apoptosis in the target cell. In some embodiments, the first adapter moiety and/or the second adapter moiety are derived from a caspase protein. In some embodiments, the first adapter moiety and the second adapter moiety are derived from caspase-9. In some embodiments, the target cell is an engineered cell adoptively transferred to an individual. In some embodiments, the target cell is a T cell expressing a chimeric antigen receptor (CAR).

In some embodiments, according to any of the systems described above where the first CID component further comprises a first adapter moiety and the second CID component further comprises a second adapter moiety, the first CID component and the second CID component are configured such that when dimerized in the presence of the IMiD to form a CID associated with a T cell, the CID is a heterodimeric CAR capable of activating the T cell upon binding a target antigen. In some embodiments, (a) the first adapter moiety comprises (i) a transmembrane domain; (ii) a cytoplasmic co-stimulatory domain; and (iii) a cytoplasmic signaling domain; and the second adapter moiety comprises an extracellular antigen-binding moiety; or (b) the second adapter moiety comprises (i) a transmembrane domain; (ii) a cytoplasmic co-stimulatory domain; and (iii) a cytoplasmic signaling domain; and the first adapter moiety comprises an extracellular antigen-binding moiety; wherein the extracellular antigen-binding moiety specifically binds to the target antigen. In some embodiments, the CID component comprising the extracellular antigen-binding moiety further comprises a secretory signal peptide. In some embodiments, (a) the first adapter moiety comprises (i) a cytoplasmic co-stimulatory domain or a cytoplasmic signaling domain; (ii) a transmembrane domain; and (iii) an extracellular antigen-binding moiety; and the second adapter moiety comprises a cytoplasmic co-stimulatory domain or a cytoplasmic signaling domain; or (b) the second adapter moiety comprises (i) a cytoplasmic co-stimulatory domain or a cytoplasmic signaling domain; (ii) a transmembrane domain; and (iii) an extracellular antigen-binding moiety; and the first adapter moiety comprises a cytoplasmic co-stimulatory domain or a cytoplasmic signaling domain; wherein the extracellular antigen-binding moiety specifically binds to the target antigen. In some embodiments, the first adapter moiety comprises (i) a cytoplasmic co-stimulatory domain or a cytoplasmic signaling domain; and (ii) a transmembrane domain; and the second adapter moiety comprises (i) a cytoplasmic co-stimulatory domain or a cytoplasmic signaling domain; and (ii) a transmembrane domain; wherein the first or second CID component further comprises an extracellular antigen-binding moiety linked to its binding moiety; and wherein the extracellular antigen-binding moiety specifically binds to the target antigen. In some embodiments, the first and second CID components together comprise a cytoplasmic co-stimulatory domain and a cytoplasmic signaling domain.

In some embodiments, according to any of the systems described above where the first CID component further comprises a first adapter moiety and the second CID component further comprises a second adapter moiety, the first CID component and the second CID component are configured such that when dimerized in the presence of the IMiD to form a CID, the CID is a heterodimeric bispecific T cell engager capable of redirecting a T cell to a target cell. In some embodiments, (a) the first adapter moiety comprises a T cell antigen-binding moiety and the second adapter moiety comprises a target cell antigen-binding moiety; or (b) the second adapter moiety comprises a T cell antigen-binding moiety and the first adapter moiety comprises a target cell antigen-binding moiety. In some embodiments, the T cell antigen-binding moiety is an antibody moiety that specifically binds to CD3. In some embodiments, the target cell antigen-binding moiety is an antibody moiety that specifically binds to a cell surface antigen associated with a diseased cell. In some embodiments, the diseased cell is a cancer cell. In some embodiments, the target cell antigen-binding moiety is an antibody moiety that specifically binds to CD19.

In some embodiments, according to any of the systems described above where the first CID component further comprises a first adapter moiety and the second CID component further comprises a second adapter moiety, the first CID component and the second CID component are configured such that when dimerized in the presence of the IMiD to form a CID associated with an immune cell, the CID is a heterodimeric signaling molecule capable of modulating activation of the immune cell. In some embodiments, the first adapter moiety comprises (i) a transmembrane domain; and (ii) a cytoplasmic co-stimulatory domain; and the second adapter moiety comprises (i) a transmembrane domain; and (ii) a cytoplasmic co-stimulatory domain. In some embodiments, the first adapter moiety further comprises a cytoplasmic signaling domain and/or the second adapter moiety further comprises a cytoplasmic signaling domain. In some embodiments, the immune cell is a T cell. In some embodiments, the T cell is a CAR T cell.

In some embodiments, according to any of the systems described above, the first CID component and the second CID component are configured such that when dimerized in the presence of the IMiD to form a CID in a cell, association of the second CID component with the first CID component allows for ubiquitination of the second CID component or a protein-of-interest (POI) associated with the second CID component. In some embodiments, dimerization of the CID results in ubiquitination of the second CID component or associated POI such that it is targeted for degradation. In some embodiments, the first CID component is endogenous cereblon in the cell. In some embodiments, the second CID component is part of a fusion protein comprising the second CID component fused to a POI. In some embodiments, the POI is a therapeutic protein, a CAR, or a cytokine. In some embodiments, the second CID component comprises a second adapter moiety comprising a POI-binding moiety capable of binding the POI. In some embodiments, the POI is a pathogen-associated protein, an aberrantly expressed endogenous protein, a CAR, or a cytokine.

In another aspect, provided herein is a method of modulating the expression of a target gene in a cell, comprising expressing the first and second CID components of a system configured for regulating target gene transcription according to any of the embodiments described above in the cell and modifying the amount of the IMiD in the cell to modulate the expression of the target gene.

In another aspect, provided herein is a method of treating a disease in an individual, comprising: (A) expressing the first and second CID components of a system configured for regulating target gene transcription according to any of the embodiments described above in target cells in an individual, wherein the expression level of the target gene in the target cells is associated with the disease; and (B) administering to the individual the IMiD in a regimen effective to treat the disease.

In another aspect, provided herein is nucleic acid encoding the first and second CID components of a system configured for regulating target gene transcription according to any of the embodiments described above.

In another aspect, provided herein is a cell comprising the first and second CID components of a system configured for regulating target gene transcription according to any of the embodiments described above.

In another aspect, provided herein is a method of controlling the survival of target cells in an individual, comprising: (A) expressing the first and second CID components of a system configured for inducing target cell death according to any of the embodiments described above in the target cells; and (B) administering to the individual the IMiD in a regimen effective to (I) kill a predetermined amount of the target cells; or (II) maintain a predetermined amount of the target cells. In some embodiments, the target cells are part of an adoptive cell therapy in the individual. In some embodiments, the target cells are CAR T cells.

In another aspect, provided herein is a method of treating a disease in an individual, comprising: (A) administering to the individual an adoptive cell therapy for the disease comprising modified cells, wherein the modified cells express the first and second CID components of a system configured for inducing target cell death according to any of the embodiments described above; and (B) administering to the individual the IMiD in a regimen effective to (I) kill a predetermined amount of the adoptively transferred cells; or (II) maintain a predetermined amount of the adoptively transferred cells. In some embodiments, the adoptive cell therapy is a CAR T cell therapy.

In another aspect, provided herein is nucleic acid encoding the first and second CID components of a system configured for inducing target cell death according to any of the embodiments described above.

In another aspect, provided herein is a cell comprising the first and second CID components of a system configured for inducing target cell death according to any of the embodiments described above. In some embodiments, the cell is part of an adoptive cell therapy. In some embodiments, the cell is a CAR T cell.

In another aspect, provided herein is a method of modulating an immune response to a target cell in an individual, comprising: (A) administering to the individual modified T cells expressing the first and second CID components of a system capable of forming a heterodimeric CAR according to any of the embodiments described above, wherein the target antigen is expressed on the surface of the target cell; and (B) administering to the individual the IMiD in a regimen effective to modulate an immune response to the target cell. In another aspect, provided herein is a method of modulating an immune response to a target cell in an individual, comprising: (A) administering to the individual modified T cells expressing the CID component of the system comprising the cytoplasmic signaling domain; (B) administering to the individual the CID component of the system comprising the extracellular antigen-binding moiety, wherein the target antigen is expressed on the surface of the target cell; and (C) administering to the individual the IMiD in a regimen effective to modulate an immune response to the target cell. In some embodiments, the regimen is effective to maintain an immune response to the target cell with fewer adverse effects in the individual as compared to a corresponding method comprising administration of CAR T cells expressing a conventional CAR comprising the corresponding CAR domains of the CID.

In another aspect, provided herein is a method of treating a disease characterized by a target cell in an individual, comprising: (A) administering to the individual modified T cells expressing the first and second CID components of a system capable of forming a heterodimeric CAR according to any of the embodiments described above, wherein the target antigen is expressed on the surface of the target cell; and (B) administering to the individual the IMiD in a regimen effective to treat the disease. In another aspect, provided herein is a method of treating a disease characterized by a target cell in an individual, comprising: (A) administering to the individual modified T cells expressing the CID component of the system comprising the cytoplasmic signaling domain; (B) administering to the individual the CID component of the system comprising the extracellular antigen-binding moiety, wherein the target antigen is expressed on the surface of the target cell; and (C) administering to the individual the IMiD in a regimen effective to treat the disease. In some embodiments, the regimen is effective to treat the disease with fewer adverse effects in the individual as compared to a corresponding method comprising administration of CAR T cells expressing a conventional CAR comprising the corresponding CAR domains of the CID.

In another aspect, provided herein is nucleic acid encoding the first and second CID components of a system capable of forming a heterodimeric CAR according to any of the embodiments described above.

In another aspect, provided herein is a T cell comprising the first and second CID components of a system capable of forming a heterodimeric CAR according to any of the embodiments described above.

In another aspect, provided herein is a T cell comprising the CID component of a system capable of forming a heterodimeric CAR according to any of the embodiments described above comprising the cytoplasmic signaling domain.

In another aspect, provided herein is a method of modulating an immune response to a target cell in an individual, comprising: (A) administering to the individual the first and second CID components of a system capable of forming a bispecific T cell engager according to any of the embodiments described above; and (B) administering to the individual the IMiD in a regimen effective to modulate an immune response to the target cell. In some embodiments, the regimen is effective to maintain an immune response to the target cell with fewer adverse effects in the individual as compared to a corresponding method comprising administration of a conventional bispecific T cell engager comprising the corresponding bispecific T cell engager domains of the CID.

In another aspect, provided herein is a method of treating a disease characterized by a target cell in an individual, comprising: (A) administering to the individual the first and second CID components of a system capable of forming a bispecific T cell engager according to any of the embodiments described above; and (B) administering to the individual the IMiD in a regimen effective to treat the disease. In some embodiments, the regimen is effective to treat the disease with fewer adverse effects in the individual as compared to a corresponding method comprising administration of a conventional bispecific T cell engager comprising the corresponding bispecific T cell engager domains of the CID.

In another aspect, provided herein is nucleic acid encoding the first and second CID components of a system capable of forming a bispecific T cell engager according to any of the embodiments described above.

In another aspect, provided herein is a method of modulating an immune response mediated by T cells in an individual, comprising: (A) expressing the first and second CID components of a system capable of modulating immune cell activation according to any of the embodiments described above in the T cells; and (B) administering to the individual the IMiD in a regimen effective to modulate an immune response mediated by the T cells. In some embodiments, the regimen is effective to maintain an immune response mediated by the T cells with fewer adverse effects in the individual as compared to a corresponding method comprising expression of a monomeric signaling molecule comprising the corresponding signaling domains of the CID in the T cells. In some embodiments, the T cells are CAR T cells.

In another aspect, provided herein is a method of treating a disease characterized by a target cell in an individual, comprising: (A) expressing the first and second CID components of a system capable of modulating immune cell activation according to any of the embodiments described above in T cells in the individual capable of recognizing and killing the target cell; and (B) administering to the individual the IMiD in a regimen effective to treat the disease. In some embodiments, the regimen is effective to treat the disease with fewer adverse effects in the individual as compared to a corresponding method comprising expression of a monomeric signaling molecule comprising the corresponding signaling domains of the CID in the T cells. In some embodiments, the T cells are CAR T cells.

In another aspect, provided herein is nucleic acid encoding the first and second CID components of a system capable of modulating immune cell activation according to any of the embodiments described above.

In another aspect, provided herein is a T cell comprising the first and second CID components of a system capable of modulating immune cell activation according to any of the embodiments described above. In some embodiments, the T cell is a CAR T cell.

In another aspect, provided herein is a method of modulating the level of a POI in a cell, comprising expressing the second CID component of a system capable of facilitating ubiquitination according to any of the embodiments described above in the cell and modifying the amount of the IMiD in the cell to modulate the level of the POI.

In another aspect, provided herein is a method of modulating an immune response to a target cell in an individual, comprising: (A) administering to the individual modified T cells expressing the second CID component of a system capable of facilitating ubiquitination according to any of the embodiments described above, wherein the POI is a CAR capable of activating the T cells upon binding a target antigen, and the target antigen is expressed on the surface of the target cell; and (B) administering to the individual the IMiD in a regimen effective to modulate an immune response to the target cell.

In another aspect, provided herein is a method of modulating an immune response to a target cell in an individual, comprising: (A) administering to the individual modified T cells expressing the second CID component of a system capable of facilitating ubiquitination according to any of the embodiments described above, wherein the POI is IL-2, IL-12, or IL-15, the T cells are CAR T cells expressing a CAR capable of activating the T cells upon binding a target antigen, and the target antigen is expressed on the surface of the target cell; and (B) administering to the individual the IMiD in a regimen effective to modulate an immune response to the target cell.

In another aspect, provided herein is a method of modulating an immune response to a target cell in an individual, comprising: (A) administering to the individual modified T cells expressing the second CID component of a system capable of facilitating ubiquitination according to any of the embodiments described above, wherein the POI is a CAR expressed in the T cells capable of activating the T cells upon binding a target antigen, and the target antigen is expressed on the surface of the target cell; and (B) administering to the individual the IMiD in a regimen effective to modulate an immune response to the target cell.

In another aspect, provided herein is a method of modulating an immune response to a target cell in an individual, comprising: (A) administering to the individual modified T cells expressing the second CID component of a system capable of facilitating ubiquitination according to any of the embodiments described above, wherein the POI is IL-2, IL-12, or IL-15, the T cells are CAR T cells expressing a CAR capable of activating the T cells upon binding a target antigen, and the target antigen is expressed on the surface of the target cell; and (B) administering to the individual the IMiD in a regimen effective to modulate an immune response to the target cell.

In another aspect, provided herein is a method of treating a disease characterized by a target cell in an individual, comprising: (A) administering to the individual modified T cells expressing the second CID component of a system capable of facilitating ubiquitination according to any of the embodiments described above, wherein the POI is a CAR capable of activating the T cells upon binding a target antigen, and the target antigen is expressed on the surface of the target cell; and (B) administering to the individual the IMiD in a regimen effective to treat the disease.

In another aspect, provided herein is a method of treating a disease characterized by a target cell in an individual, comprising: (A) administering to the individual modified T cells expressing the second CID component of a system capable of facilitating ubiquitination according to any of the embodiments described above, wherein the POI is IL-2, IL-12, or IL-15, the T cells are CAR T cells expressing a CAR capable of activating the T cells upon binding a target antigen, and the target antigen is expressed on the surface of the target cell; and (B) administering to the individual the IMiD in a regimen effective to treat the disease.

In another aspect, provided herein is a method of treating a disease characterized by a target cell in an individual, comprising: (A) administering to the individual modified T cells expressing the second CID component of a system capable of facilitating ubiquitination according to any of the embodiments described above, wherein the POI is a CAR expressed in the T cells capable of activating the T cells upon binding a target antigen, and the target antigen is expressed on the surface of the target cell; and (B) administering to the individual the IMiD in a regimen effective to treat the disease.

In another aspect, provided herein is a method of treating a disease characterized by a target cell in an individual, comprising: (A) administering to the individual modified T cells expressing the second CID component of a system capable of facilitating ubiquitination according to any of the embodiments described above, wherein the POI is IL-2, IL-12, or IL-15, the T cells are CAR T cells expressing a CAR capable of activating the T cells upon binding a target antigen, and the target antigen is expressed on the surface of the target cell; and (B) administering to the individual the IMiD in a regimen effective to treat the disease.

In another aspect, provided herein is a method of treating a disease in an individual, comprising: (A) expressing the second CID component of a system capable of facilitating ubiquitination according to any of the embodiments described above in target cells in an individual, wherein a level of the POI in the target cells above a certain threshold is associated with the disease; and (B) administering to the individual the IMiD in a regimen effective to treat the disease.

In another aspect, provided herein is nucleic acid encoding the second CID component of a system capable of facilitating ubiquitination according to any of the embodiments described above.

In another aspect, provided herein is a cell comprising the first and second CID components of a system capable of facilitating ubiquitination according to any of the embodiments described above.

### BRIEF DESCRIPTION OF THE DRAWINGS

FIG. 1A shows a graphic depiction of the design and function of an exemplary antibody-based chemically induced dimerizer (AbCID).

FIG. 1B shows a diagram of an exemplary phage selection strategy used to select IMiD-inducible Fab binders of cereblon or IMiD-binding fragments or derivatives thereof.

FIGS. 2A-2F show results for competition ELISA assays for Fab-phage clones identified from thalidomide-cereblon IMiD-binding domain selection (FIGS. 2A and 2B), lenalidomide-cereblon IMiD-binding domain selection (FIGS. 2C and 2D), and pomalidomide-cereblon IMiD-binding domain selection (FIGS. 2E and 2F).

FIG. 2G shows results for ELISA assays for Fab-phage clones TC1, TC2, LC1, PC1, and PC2 with cereblon IMiD-binding domain in the presence of thalidomide, lenalidomide, or pomalidomide as indicated.

FIGS. 3A and 3B show biolayer interferometry showing the binding properties of Fabs TC2 and PC1 to cereblon IMiD-binding domain in the presence of thalidomide, lenalidomide, or pomalidomide.

FIG. 4 shows results for IMiD-induced degradation of an eGFP fusion protein including an scFab derived from Fab-TC1 in HEK 293T cells transduced to express the fusion protein, as determined by flow cytometry for GFP fluorescence.

FIG. 5A shows a scheme where i) the scFv that typically serves as the extracellular portion of a conventional CAR construct is replaced by the first or second binding moiety of an AbCID, and ii) the other binding moiety of the AbCID is fused to the scFv, such that upon dimerization in the presence of an effective SMDA, the AbCID can function as a CAR.

FIG. 5B shows a scheme where the stimulatory and co-stimulatory domains typically found on the internal portion of a CAR construct are split and fused to either the first binding moiety or the second binding moiety of the AbCID, such that upon dimerization in the presence of an effective SMDA, the AbCID can function as a CAR.

FIG. 5C shows a scheme where the stimulatory and co-stimulatory domains typically found on the internal portion of a CAR construct are split and fused to either the first binding moiety or the second binding moiety of the AbCID through transmembrane domains, such that the first and second binding domains remain extracellular and upon dimerization in the presence of an effective SMDA, the AbCID can function as a CAR.

FIG. 5D shows a scheme where an antibody recognizing the CD3 portion of the T-Cell receptor complex is fused to the first or second binding moiety of the AbCID and an antigen-recognizing antibody is fused to the other binding moiety of the AbCID, such that upon dimerization in the presence of an effective SMDA, the AbCID can function as a bispecific T-cell engager (BiTE).

FIG. 5E shows a scheme where the transcriptional activation domain of a split human transcription factor is fused to the first or second binding domain of the AbCID and the DNA binding domain of the split human transcription factor is fused to the other binding moiety of the AbCID, such that upon dimerization in the presence of an effective SMDA, the AbCID can function in the same capacity as the transcription factor.

FIG. 5F shows a schematic of AbCID-regulated degradation of a protein-of-interest (POI) in a cell where the AbCID has a first CID component that is endogenous cereblon in the cell and a second CID component fused to the POI. Addition of an effective amount of the SMDA allows for degradation of the fusion protein including the POI as a result of ubiquitination mediated by an ubiquitin ligase complex including cereblon.

FIG. 5G shows a schematic of AbCID-regulated degradation of a protein-of-interest (POI) in a cell where the AbCID has a first CID component that is endogenous cereblon in the cell and a second CID component including a POI-binding moiety. Addition of an effective amount of the SMDA allows for degradation of the POI as a result of ubiquitination mediated by a ubiquitin ligase complex including cereblon.

### DETAILED DESCRIPTION

Temporal control over protein-protein interaction is of great importance for biological signaling. In various embodiments, the present invention provides a chemically induced dimerizer (CID) where heterodimeric subunit interaction is facilitated via a small-molecule dimerization agent (SMDA). In an exemplary CID described herein, a first component of the CID comprises a first binding moiety capable of binding an SMDA and a second component of the CID comprises a second binding moiety (e.g., an antibody (Ab) or antibody fragment) capable of specifically binding a complex between the first binding moiety and the SMDA. As will be appreciated by those of skill in the art, the second binding moiety can be any protein capable of specifically binding a first binding moiety-SMDA complex. In some embodiments, the SMDA is an immunomodulatory imide drug (IMiD) selected from the group consisting of thalidomide and analogs thereof, such as lenalidomide and pomalidomide, and first binding moiety is from a protein capable of binding to the IMiD, such as cereblon or an IMiD-binding portion or derivative thereof.

Suitable Abs for use as a second binding moiety can be generated and selected by any practical method. In an exemplary embodiment, Abs are generated from Ab-phage libraries selected against the SMDA-bound form of the first binding moiety and counter-selected against the unbound form of first binding moiety. In this way it can be ensured that the selected Ab binds more strongly to or only to the SMDA-first binding moiety complex. In a further embodiment, Abs are characterized for their binding epitope, and selected for those Abs utilizing the SMDA DA as part of its binding interface.

Thus, in an exemplary embodiment, the present invention further provides an antibody specifically binding a first binding moiety-SMDA complex. An exemplary species of these antibodies is one in which the specific binding encompasses at least a portion of both the SMDA and the first binding moiety. In a further exemplary embodiment, the portion of the SMDA and portion of the first binding moiety are those portions involved in the specific binding of the first binding moiety to the SMDA. First binding moiety-SMDA-second binding moiety dimers with these properties are also provided.

Exemplary Abs bind selectively to an IMiD-bound form of the protein cereblon by making contact with both cereblon and the IMiD.

In an exemplary embodiment, the amount of an AbCID or one of its components necessary to activate a biological system of interest is below a toxic threshold in such system of the AbCID. Thus, in such embodiments when the AbCID is utilized as a therapeutic agent, it has an acceptable therapeutic index. Similarly, when such an AbCID is used in other systems, the AbCID is not significantly toxic in the systems in which it is utilized.

AbCIDs generated from a cereblon/IMiD complex can be applied to regulate a diverse range of biological processes in living cells, including CRISPRa-mediated gene expression and CAR T-cell activation.

### Definitions

Unless defined otherwise, all technical and scientific terms used herein have the same meaning as commonly understood by one of ordinary skill in the art to which the disclosure pertains. All patents, applications, published applications and other publications referenced herein are expressly incorporated by reference in their entireties unless stated otherwise. In the event that there are a plurality of definitions for a term herein, those in this section prevail unless stated otherwise.

As used herein, “a” or “an” may mean one or more than one.

“About” has its plain and ordinary meaning when read in light of the specification, and may be used, for example, when referring to a measurable value and may be meant to encompass variations of ±20% or ±10%, more preferably ±5%, even more preferably ±1%, and still more preferably ±0.1% from the specified value.

By “amino acid” and “amino acid identity”, as used herein, is meant one of the 20 naturally occurring amino acids or any non-natural analogues that may be present at a specific, defined position. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e. “analogs”, such as peptoids (see Simon et al., PNAS USA 89(20):9367 (1992)) particularly when peptides are to be administered to a patient. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

By “amino acid modification”, as used herein, is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA. The preferred amino acid modification herein is a substitution.

By “amino acid substitution” or “substitution”, as used herein, is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine, wherein numbering is according to the EU system as in Kabat. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion”, as used herein, is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion”, as used herein, is meant the removal of an amino acid sequence at a particular position in a parent polypeptide sequence. For example, E233- or E233#, E233( ) or E233del designates a deletion of glutamic acid at position 233. Additionally, EDA233- or EDA233# designates a deletion of the sequence GluAspAla that begins at position 233.

As used herein, “polypeptide”, refers to at least two covalently attached amino acids, which includes proteins, polypeptides, oligopeptides and peptides. The peptidyl group may comprise naturally occurring amino acids and peptide bonds, or synthetic peptidomimetic structures, i.e., “analogs”, such as peptoids (see Simon et al., PNAS USA 89(20):9367 (1992), entirely incorporated by reference). The amino acids may either be naturally occurring or synthetic (e.g., not an amino acid that is coded for by DNA); as will be appreciated by those in the art. For example, homo-phenylalanine, citrulline, ornithine and noreleucine are considered synthetic amino acids for the purposes of the invention, and both D- and L- (R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “parent polypeptide” or “precursor polypeptide” (including Fc parent or precursors), as used herein, is meant a polypeptide that is subsequently modified to generate a variant. Said parent polypeptide may be a naturally occurring polypeptide, or a variant or engineered version of a naturally occurring polypeptide.

“Antibody” as used herein is meant to include full length antibodies and antibody fragments, and may refer to a natural antibody from any organism, an engineered antibody, or an antibody generated recombinantly for experimental, therapeutic, or other purposes as further defined below. Thus, “antibody” includes both polyclonal and monoclonal antibody (mAb). Antibody fragments are known in the art and include, without limitation, Fab, Fab′, F(ab′)2, Fcs or other antigen-binding subsequences of antibodies, such as, single chain antibodies (scFab and scFv for example), chimeric antibodies, etc., either produced by the modification of whole antibodies or those synthesized de novo using recombinant DNA technologies.

By “Fv” or “Fv fragment” or “Fv region” as used herein is meant a polypeptide that comprises the VL and VH domains of a single antibody. As will be appreciated by those in the art, these generally are made up of two chains, or can be combined (generally with a linker as discussed herein) to form a scFv.

By “single chain Fv” or “scFv” herein is meant a variable heavy (VH) domain covalently attached to a variable light (VL) domain, generally using a scFv linker as discussed herein, to form a scFv or scFv domain. A scFv domain can be in either orientation from N- to C-terminus (VH-linker-VL or VL-linker-VH).

By “single chain Fab” or “scFab” as used herein is meant a variable heavy (VH) domain covalently attached to a constant heavy (CH) domain, which is in turn attached to a constant light (CL) domain attached to a variable light (VL) domain, generally using a scFab linker as discussed herein, to form a scFab or scFab domain.

By “variable region”, as used herein, is meant the region of an immunoglobulin that comprises one or more Ig domains substantially encoded by any of the Vic, Vλ, VL and/or VH genes that make up the kappa, lambda, and heavy and light chain immunoglobulin genetic loci respectively.

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is selected from CD-19 and CD-3. In an exemplary embodiment, CD-x is a target for a CAR T-cell.

By “antigen binding domain” or “ABD” herein is meant a set of six Complementary Determining Regions (CDRs) that, when present as part of a polypeptide sequence, specifically binds a target antigen as discussed herein. Thus, a “checkpoint antigen binding domain” binds a target checkpoint antigen as outlined herein. As is known in the art, these CDRs are generally present as a first set of variable heavy CDRs (vhCDRs or VHCDRs) and a second set of variable light CDRs (vhCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv or scFab format, the vh and vl domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used. An exemplary second binding domain recognizes and specifically binds to a first binding moiety-SMDA complex. Exemplary CDRs of exemplary second binding moiety molecules of the invention are provided in Table 1, and exemplary heavy chain and light chain variable domain scaffolds are provided in SEQ ID NOs: 23 and 24, respectively.

By “wild type” or “WT” herein is meant an amino acid sequence or a nucleotide sequence that is found in nature, including allelic variations.

By “variant polypeptide” as used herein is meant a polypeptide sequence that differs from that of a parent polypeptide sequence by virtue of at least one amino acid modification. Modifications can include substitutions, deletions, and additions. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% identity with a parent polypeptide sequence, and most preferably at least about 90% identity, more preferably at least about 95% identity. Accordingly, by “Fc variant” as used herein is meant an Fc sequence that differs from that of a parent Fc sequence by virtue of at least one amino acid modification. Similarly, an exemplary “inactive VL domain” or “inactive VH domain” is a variant of a parent VL or VH polypeptide.

In some embodiments, the AbCIDs and/or the polypeptide components of the invention are “isolated” or “substantially pure” polypeptides. “Isolated” or “substantially pure”, when used to describe the polypeptides disclosed herein, means a polypeptide that has been identified, separated and/or recovered from a component of its production environment. Preferably, the polypeptide is free or substantially free of association with all other components from its production environment. Contaminant components of its production environment, such as that resulting from recombinant transfected cells, are materials that would typically interfere with therapeutic or other uses for the polypeptide, and may include enzymes, hormones, and other proteinaceous or non-proteinaceous solutes. The desired polypeptide in the production medium may constitute at least about 5%, at least about 25% or at least about 50% by weight of the total polypeptide the medium.

Exemplary isolated polypeptides and AbCIDs including the polypeptides of the invention are substantially or essentially free from components, which are used to produce the material. For peptides of the invention, the term “isolated” refers to material that is substantially or essentially free from components, which normally accompany the material in the mixture used to prepare the peptide. “Isolated” and “pure” are used interchangeably. Typically, isolated polypeptides of the invention have a level of purity preferably expressed as a range. The lower end of the range of purity for the polypeptide constructs is about 60%, about 70% or about 80% and the upper end of the range of purity is about 70%, about 80%, about 90% or more than about 90%.

When the polypeptides are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

In various embodiments, the first binding moiety binds to SMDA through one or more binding domains. According to the present invention, binding domains are components of one or more polypeptides. Such polypeptides may include proteinaceous parts and non-proteinaceous parts (e.g. chemical linkers or chemical cross-linking agents such as glutaraldehyde).

As used herein, the term “tumor” refers to all neoplastic cell growth and proliferation, whether malignant or benign, and all cancerous and pre-cancerous cells and tissues.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The terms “targeting moiety” and “targeting agent”, as used herein, refer to species that will selectively localize in a particular tissue or region of the body.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is linked to a carrier, e.g., multivalent agents. Therapeutic moiety also includes peptides, and constructs that include peptides. “Therapeutic moiety” thus means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is linked to a carrier, e.g., multivalent agents.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-α. Conjugates include, but are not limited to those formed between a therapeutic protein and a glycoprotein of the invention. A representative conjugate is that formed between PSGL-1 and TNF-α.

The term “half-life” or “t½”, as used herein in the context of administering a SMDA or a SMDA-protein complex to a patient, is defined as the time required for plasma concentration of the substance administered to a patient to be reduced by one half. There may be more than one half-life associated with the administered species depending on multiple clearance mechanisms, redistribution, and other mechanisms well known in the art. Usually, alpha and beta half-lives are defined such that the alpha phase is associated with redistribution, and the beta phase is associated with clearance. However, with protein drugs that are, for the most part, confined to the bloodstream, there can be at least two clearance half-lives. For some glycosylated peptides, rapid beta phase clearance may be mediated via receptors on macrophages, or endothelial cells that recognize terminal galactose, N-acetylgalactosamine, N-acetylglucosamine, mannose, or fucose. Slower beta phase clearance may occur via renal glomerular filtration for molecules with an effective radius <2 nm (approximately 68 kD) and/or specific or non-specific uptake and metabolism in tissues. PEGylation may cap terminal sugars (e.g. galactose or N-acetylgalactosamine) and thereby block rapid alpha phase clearance via receptors that recognize these sugars. It may also confer a larger effective radius and thereby decrease the volume of distribution and tissue uptake, thereby prolonging the late beta phase. Thus, the precise impact of PEGylation on alpha phase and beta phase half-lives will vary depending upon the size, state of glycosylation, and other parameters, as is well known in the art. Further explanation of “half-life” is found in Pharmaceutical Biotechnology (1997, DFA Crommelin and RD Sindelar, eds., Harwood Publishers, Amsterdam, pp 101-120).

### Immunomodulatory Imide Drugs (IMiDs)

In various embodiments described herein, an IMiD is employed. In some embodiments, the IMiD is thalidomide, which has the following chemical structure:

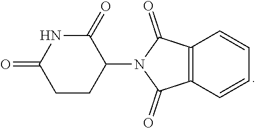 [SMILES: O=C1CCC(N2C(=O)c3ccccc3C2=O)C(=O)N1]

In some embodiments, the IMiD is lenalidomide, which has the following chemical structure:

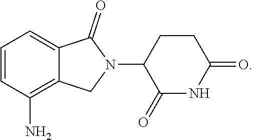 [SMILES: Nc1cccc2c1CN(C1CCC(=O)NC1=O)C2=O]

In some embodiments, the IMiD is pomalidomide, which has the following chemical structure:

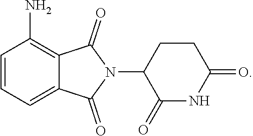 [SMILES: Nc1cccc2c1C(=O)N(C1CCC(=O)NC1=O)C2=O]

### AbCIDs

In various embodiments, provided herein are systems, compositions, and methods to achieve temporal SMDA-mediated control over the formation of a CID where the second binding moiety of the CID is an antibody moiety, such a CID referred to herein as an “AbCID.” In exemplary embodiments, these SMDA-induced interactions serve as molecular switches, which lead to the transmissions of cellular signals. These signals can be programmed to be pro-survival or pro-death depending on the therapeutic need and application. An exemplary signal activates T cells.

The present invention can be applied to a number of applications, e.g., engineered T-cell therapy and control of gene expression, e.g., using CRISPR.

Chimeric antigen receptor (CAR) T-Cell therapy is showing great promise in treating a number of cancers. Unfortunately, overly activated T-Cells often have negative side effects, limiting the use of this therapy. The present invention is exemplified by application to CAR T-Cells in two ways: For example, one of the key problems with CAR-T-cells is that they have a long half-life in the body. Thus there is an interest in removing them once they have had the therapeutic effect. The present invention is of use to build a SMDA-induced death switch so CAR T-cells can be eliminated from a subject being treated with same when desired. For example, the invention can be utilized to incorporate a chemically inducible death switch into a CAR T system so that if the T-Cells become toxic to the patient, a SMDA can be administered that will selectively trip the switch and kill the engineered T-Cells.

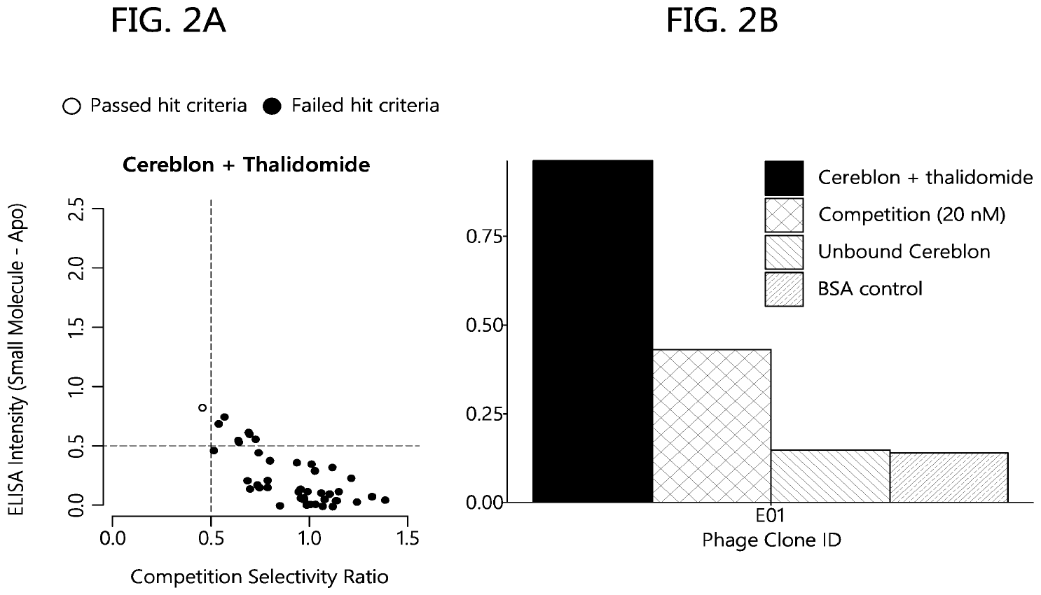

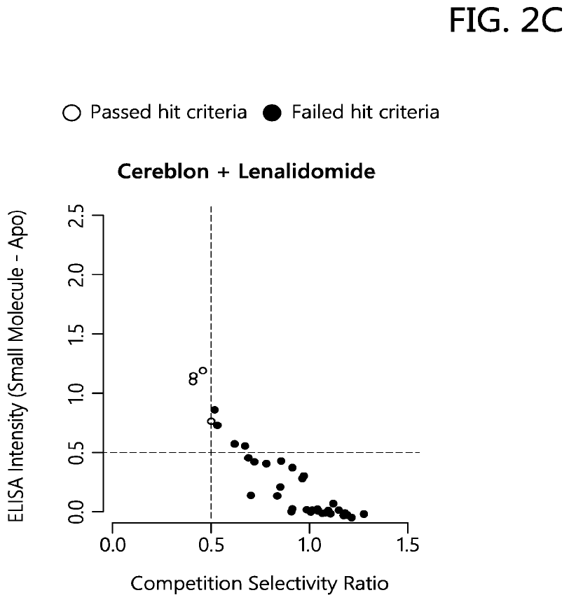

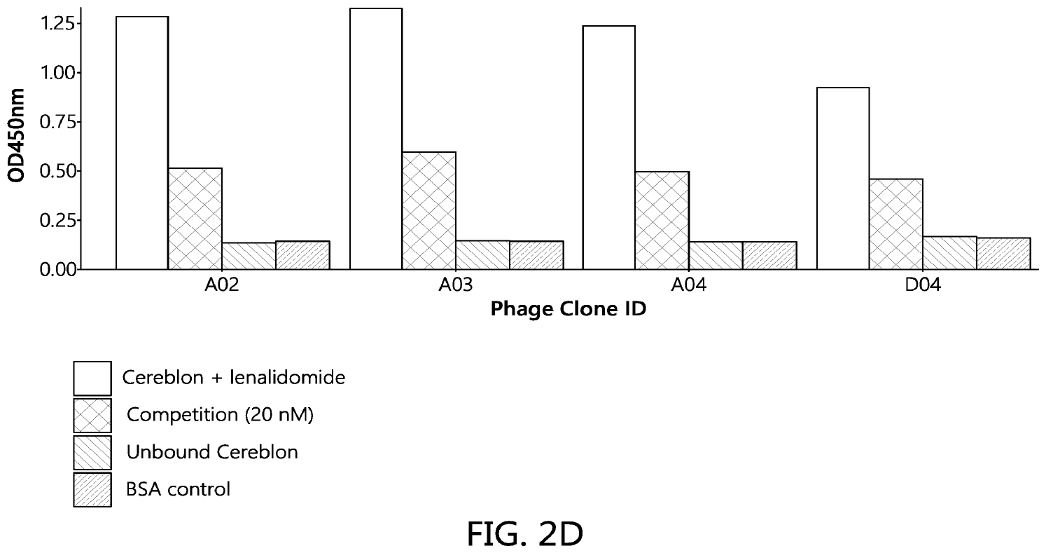

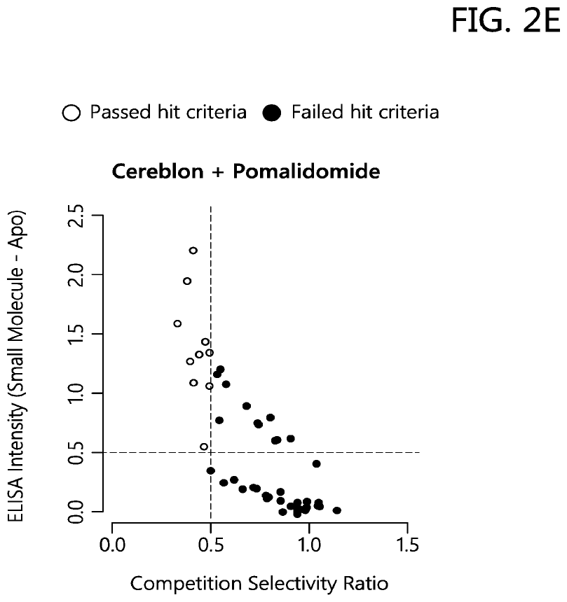

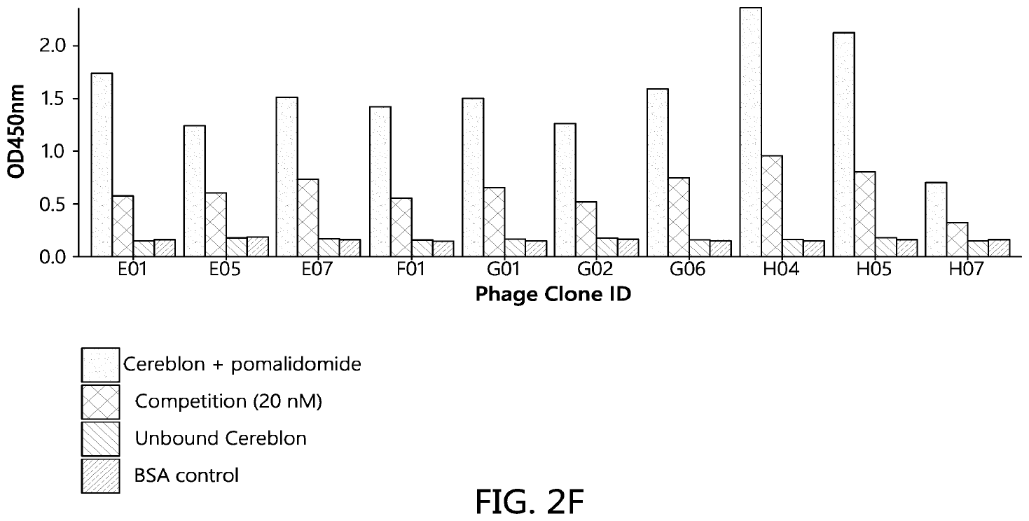

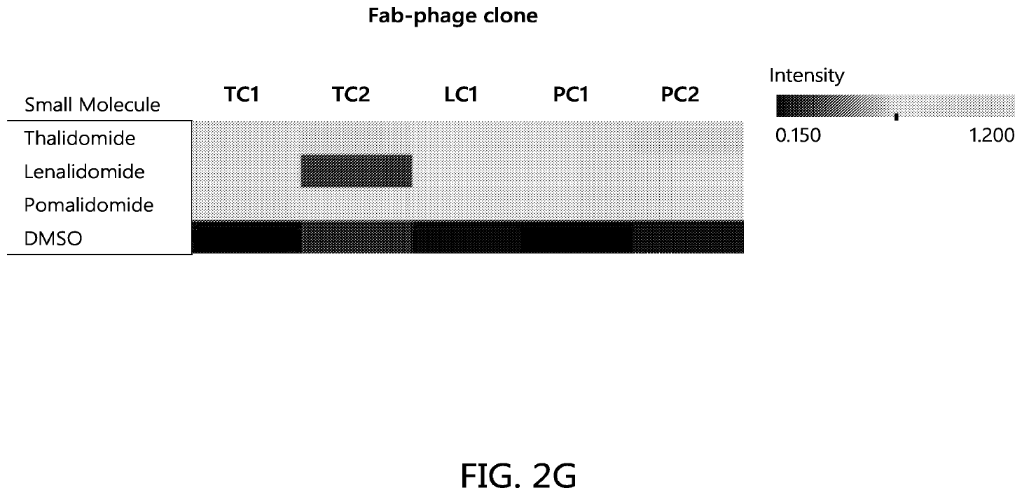

The instant invention also provides an AbCID comprising an activator and antigen recruiter for CAR T-Cells. In an exemplary embodiment, a first component of the AbCID comprises a CAR where the scFv portion of the CAR is replaced with cereblon or an IMiD-binding portion or derivative thereof (first binding moiety) capable of binding an IMiD (e.g., thalidomide, lenalidomide, or pomalidomide), and a second component of the AbCID comprises a bispecific antibody comprising a first antibody moiety specific for a complex between the first CID component and the IMiD (second binding moiety) and a second antibody moiety specific for a target antigen (e.g., a disease-associated antigen, such as a cancer-associate antigen) presented on the surface of a target cell (e.g., a diseased cell, such as a cancer cell). Provision of the IMiD to a T-cell comprising the first CID component allows for dimerization of the first CID component on the T-cell to the second CID component present in the environment surrounding the T-cell, allowing the T-cell to be activated when contacted with the target antigen (FIG. 2). As the IMiD is titratable and reversible, this allows for the modulation of the T-Cells' activation and the duration of their effect, greatly improving the therapeutic index and thus safety of this therapeutic approach.

One aspect of the present disclosure provides a dimer formed through an AbCID, which incorporates a RNA-guided endonucleases comprising at least one nuclear localization signal, which permits entry of the endonuclease into the nuclei of eukaryotic cells and embryos such as, for example, non-human one cell embryos. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences. Provided herein are isolated RNA-guided endonucleases, isolated nucleic acids (i.e., RNA or DNA) encoding the RNA-guided endonucleases, vectors comprising nucleic acids encoding the RNA-guided endonucleases, and protein-RNA complexes comprising the RNA-guided endonuclease plus a guide RNA. Also of use in methods of the invention are catalytically inactive variants of Cas-9.

The RNA-guided endonuclease can be derived from a clustered regularly interspersed short palindromic repeats (CRISPR)/CRISPR-associated (Cas) system. The CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14charged, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein. As will be appreciated by those of skill in the art, either the first binding moiety or second binding moiety of a CID can be fused to other DNA-binding proteins besides Cas9, e.g., Zinc-finger proteins.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek et al., Science, 2012, 337: 816-821). In some embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a nickase. Likewise, a histidine to alanine (H840A or H839A) conversion in a HNH domain converts the Cas9-derived protein into a nickase. Each nuclease domain can be modified using well-known methods, such as site-directed mutagenesis, PCR-mediated mutagenesis, and total gene synthesis, as well as other methods known in the art. The compounds and methods of the present invention can also be applied to regulate genetic circuits using proteins other than CRISPR-derived proteins, e.g., TALEN, Zinc Finger Proteins, etc.

In an exemplary embodiment, the AbCID forms a dimer having an in vivo half-life, which is longer than any subset of its components when administered to a subject.

In various embodiments, the AbCID forms a dimer between a first protein conjugate comprising a member selected from a first therapeutic moiety, a first targeting moiety, a first detectable moiety and a first combination thereof; and a second protein conjugate comprising a second therapeutic moiety, a second targeting moiety, a second detectable moiety and a second combination thereof. In an exemplary embodiment, this dimer is formed in vivo following administration of the SMDA or the first binding moiety-SMDA complex to a patient in need of therapeutic intervention by such administration.

Exemplary embodiments of the invention utilize antibodies as one or both of the first and second binding moieties of a CID. As is discussed herein, the term “antibody” is used in its most general sense. Antibodies finding use in the present invention can be of a number of formats as described herein, including traditional full antibodies as well as antibody derivatives, fragments and mimetics, as described herein, depicted in the figures and generally described in the art.

Thus, in an exemplary embodiment, the invention provides an antibody capable of specifically binding to a complex formed between first binding moiety and its cognate SMDA binding partner. In various embodiments, the antibody specifically binds to at least a portion of the complexed SM. In various embodiments, the antibody binds to at least a portion of the first binding moiety complexed with SM. In an exemplary embodiment, the antibody binds simultaneously to both at least a portion of the complexed SMDA and at least a portion of the first binding moiety bound to SM. In various embodiments, one or both of these binding modalities is a component of the specific binding of the antibody to the first binding moiety-SMDA complex. In an exemplary embodiment, the second binding moiety binds to the first binding moiety-SMDA complex at a position or surface having substantial solvent access. There are several approaches useful to define surface accessibility of the SMDA in the first binding moiety-SMDA complex. For example, a crystal structure of the first binding moiety-SMDA complex can be used to calculate the solvent accessibility of the SMDA in the complex. Other methods of inferring solvent accessibility of the bound SMDA include NMR methods to either determine their structure, or solvent interactions with the SM. Another measure of surface accessibility is the amount of surface area of the first binding moiety-SMDA complex exposed to solvent. Exemplary ranges for a solvent exposed surface area are less than about 1000 Å², e.g., less than about 500 Å², less than about 100 Å², less than about 50 Å². In exemplary embodiments, the solvent accessible surface is from about 1 to about 20 Å², e.g., from about 1 to about 10 Å², or from about 10-20 Å².

Traditional full antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention provides dimers incorporating bispecific antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

“Isotype” as used herein is meant any of the subclasses of immunoglobulins defined by the chemical and antigenic characteristics of their constant regions. It should be understood that therapeutic antibodies can also comprise hybrids of isotypes and/or subclasses. For example, as shown in US Publication 2009/0163699, incorporated by reference, the present invention covers pI engineering of IgG1/G2 hybrids.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. An exemplary epitope is that which is formed by the complex formed by the binding of the SMDA to the first binding moiety.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. Exemplary epitopes of use in the compounds and methods of the invention include those structures formed by the binding of SMDA by the first binding moiety.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

### Exemplary AbCIDs

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

TABLE 1
Fab-phage clone CDRs

| | HC-CDR1 | HC-CDR2 | HC-CDR3 | LC-CDR1 | LC-CDR2 | LC-CDR3 |
|---|---|---|---|---|---|---|
| | (SEQ ID | (SEQ ID | (SEQ ID | (SEQ ID | (SEQ ID | (SEQ ID |
| Clone | NO) | NO) | NO) | NO) | NO) | NO) |
| Fab-TC1 | ISSYSI | SISPSYGYT | SYYWQYYYQ | RASQSV | SASSLY | GSQSQMPF |
| | (SEQ ID | S | FGYPFGF | SSAVA | S (SEQ | (SEQ ID |
| | NO: 1) | (SEQ ID | (SEQ ID | (SEQ | ID NO: | NO: 16) |
| | | NO: 6) | NO: 11) | ID NO: | 22) | |
| Fab-TC2 | VSSSSI | SIYSYYGST | TDSWYYYRY | 21) | | YSWWWMPI |
| | (SEQ ID | Y | GGM | | | (SEQ ID |
| | NO: 2) | (SEQ ID | (SEQ ID | | | NO: 17) |
| | | NO: 7) | NO: 12) | | | |
| Fab-LC1 | FSSSSI | YISSSSGST | SEYPYSYWY | | | WGYYLI |
| | (SEQ ID | S | MYGYPVGGF | | | (SEQ ID |
| | NO: 3) | (SEQ ID | (SEQ ID | | | NO: 18) |
| | | NO: 8) | NO: 13) | | | |
| Fab-PC1 | VSSYSI | SISSYSGST | SYFVEYYYY | | | SSYSYLF |
| | (SEQ ID | S | YGWPWGL | | | (SEQ ID |
| | NO: 4 ) | (SEQ ID | (SEQ ID | | | NO: 19) |
| | | NO: 9) | NO: 14) | | | |
| Fab-PC2 | VSYSSI | SIYSYSGST | SSYSYWYYI | | | SDSMPV |
| | (SEQ ID | S | MYGYWFAM | | | (SEQ ID |
| | NO: 5) | (SEQ ID | (SEQ ID | | | NO: 20) |
| | | NO: 10) | NO: 15) | | | |

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety comprising i) a heavy chain variable domain comprising an HC-CDR1 comprising the amino acid sequence of SEQ ID NO: 1 or a variant thereof having at least about 85% sequence identity to SEQ ID NO: 1; an HC-CDR2 comprising the amino acid sequence of SEQ ID NO: 6 or a variant thereof having at least about 85% sequence identity to SEQ ID NO: 6; and an HC-CDR3 comprising the amino acid sequence of SEQ ID NO: 11 or a variant thereof having at least about 85% sequence identity to SEQ ID NO: 11, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 16 or a variant thereof having at least about 85% sequence identity to SEQ ID NO: 16. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety comprising i) a heavy chain variable domain comprising an HC-CDR1 comprising the amino acid sequence of SEQ ID NO: 2 or a variant thereof having at least about 85% sequence identity to SEQ ID NO: 2; an HC-CDR2 comprising the amino acid sequence of SEQ ID NO: 7 or a variant thereof having at least about 85% sequence identity to SEQ ID NO: 7; and an HC-CDR3 comprising the amino acid sequence of SEQ ID NO: 12 or a variant thereof having at least about 85% sequence identity to SEQ ID NO: 12, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 17 or a variant thereof having at least about 85% sequence identity to SEQ ID NO: 17. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety comprising i) a heavy chain variable domain comprising an HC-CDR1 comprising the amino acid sequence of SEQ ID NO: 3 or a variant thereof having at least about 85% sequence identity to SEQ ID NO: 3; an HC-CDR2 comprising the amino acid sequence of SEQ ID NO: 8 or a variant thereof having at least about 85% sequence identity to SEQ ID NO: 8; and an HC-CDR3 comprising the amino acid sequence of SEQ ID NO: 13 or a variant thereof having at least about 85% sequence identity to SEQ ID NO: 13, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 18 or a variant thereof having at least about 85% sequence identity to SEQ ID NO: 18. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety comprising i) a heavy chain variable domain comprising an HC-CDR1 comprising the amino acid sequence of SEQ ID NO: 4 or a variant thereof having at least about 85% sequence identity to SEQ ID NO: 4; an HC-CDR2 comprising the amino acid sequence of SEQ ID NO: 9 or a variant thereof having at least about 85% sequence identity to SEQ ID NO: 9; and an HC-CDR3 comprising the amino acid sequence of SEQ ID NO: 14 or a variant thereof having at least about 85% sequence identity to SEQ ID NO: 14, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 19 or a variant thereof having at least about 85% sequence identity to SEQ ID NO: 19. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, according to any of the AbCIDs described herein, the AbCID comprises (a) a first CID component comprising a first binding moiety comprising cereblon or an IMiD-binding fragment or derivative thereof; and (b) a second CID component comprising a second binding moiety, wherein the second binding moiety is an antibody moiety comprising i) a heavy chain variable domain comprising an HC-CDR1 comprising the amino acid sequence of SEQ ID NO: 5 or a variant thereof having at least about 85% sequence identity to SEQ ID NO: 5; an HC-CDR2 comprising the amino acid sequence of SEQ ID NO: 10 or a variant thereof having at least about 85% sequence identity to SEQ ID NO: 10; and an HC-CDR3 comprising the amino acid sequence of SEQ ID NO: 15 or a variant thereof having at least about 85% sequence identity to SEQ ID NO: 15, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 20 or a variant thereof having at least about 85% sequence identity to SEQ ID NO: 20. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

### Systems

In some embodiments, provided herein is a system comprising (a) a first chemically induced dimer (CID) component comprising a first binding moiety capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety that specifically binds to the complex between the first CID component and the IMiD, or a second nucleic acid encoding polypeptide components of the second CID component. In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the first binding moiety comprises cereblon or an IMiD-binding fragment or derivative thereof. In some embodiments, the IMiD is thalidomide. In some embodiments, the IMiD is lenalidomide. In some embodiments, the IMiD is pomalidomide.

Thus, in some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety that specifically binds to the complex between the first CID component and the IMiD, or a second nucleic acid encoding polypeptide components of the second CID component. In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide.

In some embodiments, provided herein is a system comprising (a) a first CID component comprising a first binding moiety comprising cereblon or a thalidomide-binding fragment or derivative thereof, wherein the first binding moiety is capable of interacting with thalidomide to form a complex between the first CID component and thalidomide, or a first nucleic acid encoding polypeptide components of the first CID component; and (b) a second CID component comprising a second binding moiety that specifically binds to the complex between the first CID component and thalidomide, or a second nucleic acid encoding polypeptide components of the second CID component. In some embodiments, the second binding moiety specifically binds to a site of the complex comprising at least a portion of the first binding moiety and a portion of thalidomide. In some embodiments, the first CID component further comprises a first adapter moiety linked to the first binding moiety and/or the second CID component further comprises a second adapter moiety linked to the second binding moiety. In some embodiments, the system further comprises thalidomide, wherein the second CID component is bound to a complex between thalidomide and the first CID component at a site of the complex comprising at least a portion of thalidomide and a portion of the first binding moiety. In some embodiments, the site of the complex comprising at least a portion of thalidomide and a portion of the first binding moiety is an interface between thalidomide and a binding site of the first binding moiety for thalidomide, comprising at least one atom of thalidomide and at least one atom of the first binding moiety.

In some embodiments, provided herein is a system comprising (a) a first CID component comprising a first binding moiety comprising cereblon or a lenalidomide-binding fragment or derivative thereof, wherein the first binding moiety is capable of interacting with lenalidomide to form a complex between the first CID component and lenalidomide, or a first nucleic acid encoding polypeptide components of the first CID component; and (b) a second CID component comprising a second binding moiety that specifically binds to the complex between the first CID component and lenalidomide, or a second nucleic acid encoding polypeptide components of the second CID component. In some embodiments, the second binding moiety specifically binds to a site of the complex comprising at least a portion of the first binding moiety and a portion of lenalidomide. In some embodiments, the first CID component further comprises a first adapter moiety linked to the first binding moiety and/or the second CID component further comprises a second adapter moiety linked to the second binding moiety. In some embodiments, the system further comprises lenalidomide, wherein the second CID component is bound to a complex between lenalidomide and the first CID component at a site of the complex comprising at least a portion of lenalidomide and a portion of the first binding moiety. In some embodiments, the site of the complex comprising at least a portion of lenalidomide and a portion of the first binding moiety is an interface between lenalidomide and a binding site of the first binding moiety for lenalidomide, comprising at least one atom of lenalidomide and at least one atom of the first binding moiety.

In some embodiments, provided herein is a system comprising (a) a first CID component comprising a first binding moiety comprising cereblon or a pomalidomide-binding fragment or derivative thereof, wherein the first binding moiety is capable of interacting with pomalidomide to form a complex between the first CID component and pomalidomide, or a first nucleic acid encoding polypeptide components of the first CID component; and (b) a second CID component comprising a second binding moiety that specifically binds to the complex between the first CID component and pomalidomide, or a second nucleic acid encoding polypeptide components of the second CID component. In some embodiments, the second binding moiety specifically binds to a site of the complex comprising at least a portion of the first binding moiety and a portion of pomalidomide. In some embodiments, the first CID component further comprises a first adapter moiety linked to the first binding moiety and/or the second CID component further comprises a second adapter moiety linked to the second binding moiety. In some embodiments, the system further comprises pomalidomide, wherein the second CID component is bound to a complex between pomalidomide and the first CID component at a site of the complex comprising at least a portion of pomalidomide and a portion of the first binding moiety. In some embodiments, the site of the complex comprising at least a portion of pomalidomide and a portion of the first binding moiety is an interface between pomalidomide and a binding site of the first binding moiety for pomalidomide, comprising at least one atom of pomalidomide and at least one atom of the first binding moiety.

In some embodiments, according to any of the systems described herein wherein the second binding moiety specifically binds to a site of the complex comprising at least a portion of an IMiD (e.g., thalidomide, lenalidomide, or thalidomide) and a portion of the first binding moiety, the second binding moiety is an antibody moiety that specifically binds to a chemical-epitope comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and one atom of the first binding moiety.

In some embodiments, according to any of the systems described herein, the second CID component binds to the complex of the first CID component and the IMiD with a dissociation constant (K_{d}) no more than about 1/250 times (such as no more than about any of 1/300, 1/350, 1/400, 1/450, 1/500, 1/600, 1/700, 1/800, 1/900, 1/1000, 1/1100, 1/1200, 1/1300, 1/1400, or 1/1500 times, or less) its K_{d} for binding to each of the free first CID component and the free IMiD.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety capable of specifically binding to a complex between the first binding moiety and an IMiD (e.g., thalidomide, lenalidomide, or pomalidomide), or a second nucleic acid encoding polypeptide components of the second CID component, wherein the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety comprising i) a heavy chain variable domain comprising an HC-CDR1 comprising the amino acid sequence of SEQ ID NO: 1 or a variant thereof having at least about 85% sequence identity to SEQ ID NO: 1; an HC-CDR2 comprising the amino acid sequence of SEQ ID NO: 6 or a variant thereof having at least about 85% sequence identity to SEQ ID NO: 6; and an HC-CDR3 comprising the amino acid sequence of SEQ ID NO: 11 or a variant thereof having at least about 85% sequence identity to SEQ ID NO: 11, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 16 or a variant thereof having at least about 85% sequence identity to SEQ ID NO: 16. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety comprising i) a heavy chain variable domain comprising an HC-CDR1 comprising the amino acid sequence of SEQ ID NO: 2 or a variant thereof having at least about 85% sequence identity to SEQ ID NO: 2; an HC-CDR2 comprising the amino acid sequence of SEQ ID NO: 7 or a variant thereof having at least about 85% sequence identity to SEQ ID NO: 7; and an HC-CDR3 comprising the amino acid sequence of SEQ ID NO: 12 or a variant thereof having at least about 85% sequence identity to SEQ ID NO: 12, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 17 or a variant thereof having at least about 85% sequence identity to SEQ ID NO: 17. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety comprising i) a heavy chain variable domain comprising an HC-CDR1 comprising the amino acid sequence of SEQ ID NO: 3 or a variant thereof having at least about 85% sequence identity to SEQ ID NO: 3; an HC-CDR2 comprising the amino acid sequence of SEQ ID NO: 8 or a variant thereof having at least about 85% sequence identity to SEQ ID NO: 8; and an HC-CDR3 comprising the amino acid sequence of SEQ ID NO: 13 or a variant thereof having at least about 85% sequence identity to SEQ ID NO: 13, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 18 or a variant thereof having at least about 85% sequence identity to SEQ ID NO: 18. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety comprising i) a heavy chain variable domain comprising an HC-CDR1 comprising the amino acid sequence of SEQ ID NO: 4 or a variant thereof having at least about 85% sequence identity to SEQ ID NO: 4; an HC-CDR2 comprising the amino acid sequence of SEQ ID NO: 9 or a variant thereof having at least about 85% sequence identity to SEQ ID NO: 9; and an HC-CDR3 comprising the amino acid sequence of SEQ ID NO: 14 or a variant thereof having at least about 85% sequence identity to SEQ ID NO: 14, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 19 or a variant thereof having at least about 85% sequence identity to SEQ ID NO: 19. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a system comprising (a) a first CID component comprising a first binding moiety comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a second binding moiety, wherein the second binding moiety is an antibody moiety comprising i) a heavy chain variable domain comprising an HC-CDR1 comprising the amino acid sequence of SEQ ID NO: 5 or a variant thereof having at least about 85% sequence identity to SEQ ID NO: 5; an HC-CDR2 comprising the amino acid sequence of SEQ ID NO: 10 or a variant thereof having at least about 85% sequence identity to SEQ ID NO: 10; and an HC-CDR3 comprising the amino acid sequence of SEQ ID NO: 15 or a variant thereof having at least about 85% sequence identity to SEQ ID NO: 15, and ii) a light chain variable domain comprising an LC-CDR1 comprising the amino acid sequence of SEQ ID NO: 21 or a variant thereof having at least about 85% sequence identity to SEQ ID NO: 21; an LC-CDR2 comprising the amino acid sequence of SEQ ID NO: 22 or a variant thereof having at least about 85% sequence identity to SEQ ID NO: 22; and an LC-CDR3 comprising the amino acid sequence of SEQ ID NO: 20 or a variant thereof having at least about 85% sequence identity to SEQ ID NO: 20. In some embodiments, the antibody moiety is capable of specifically binding to a complex between the first binding moiety and an IMiD (e.g., thalidomide, lenalidomide, or pomalidomide). In some embodiments, the second binding moiety specifically binds to a site of the complex comprising at least a portion of the first binding moiety and a portion of the IMiD. In some embodiments, the first CID component further comprises a first adapter moiety linked to the first binding moiety and/or the second CID component further comprises a second adapter moiety linked to the second binding moiety. In some embodiments, the system further comprises the IMiD, wherein the second CID component is bound to a complex between the IMiD and the first CID component at a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and at least one atom of the first binding moiety. In some embodiments, the IMiD is thalidomide. In some embodiments, the IMiD is lenalidomide. In some embodiments, the IMiD is pomalidomide. In some embodiments, the first binding moiety comprises the amino acid sequence of SEQ ID NO: 25 or a variant thereof having at least 85% identity to the amino acid sequence of SEQ ID NO: 25.

### Transcriptional Regulator

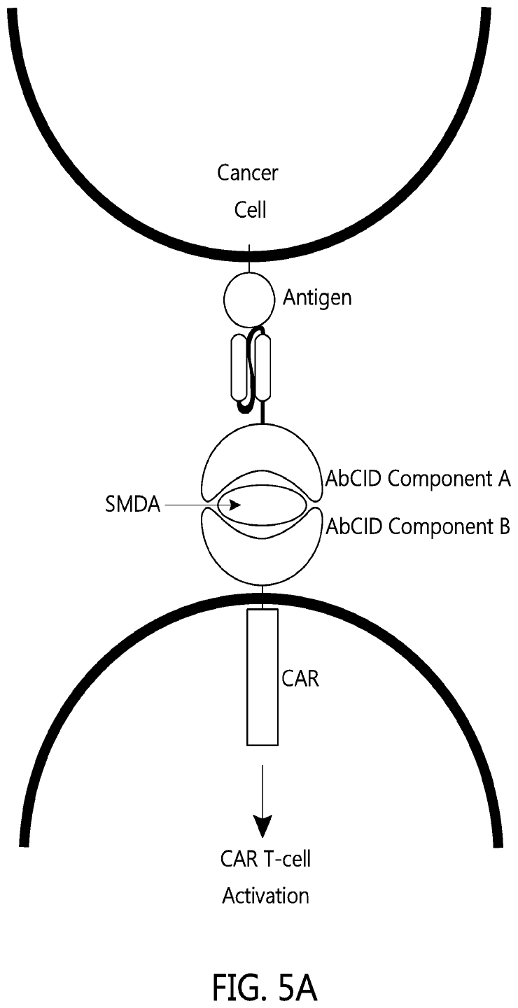

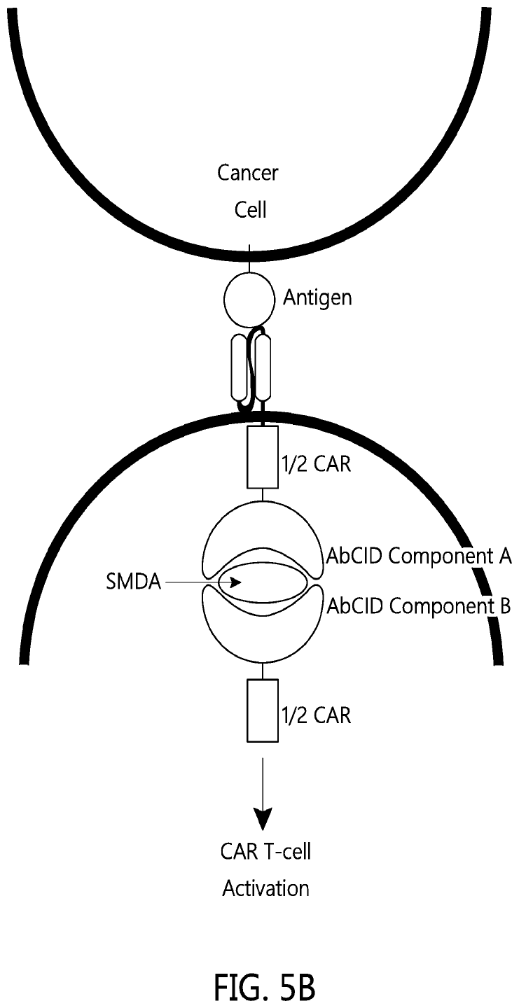

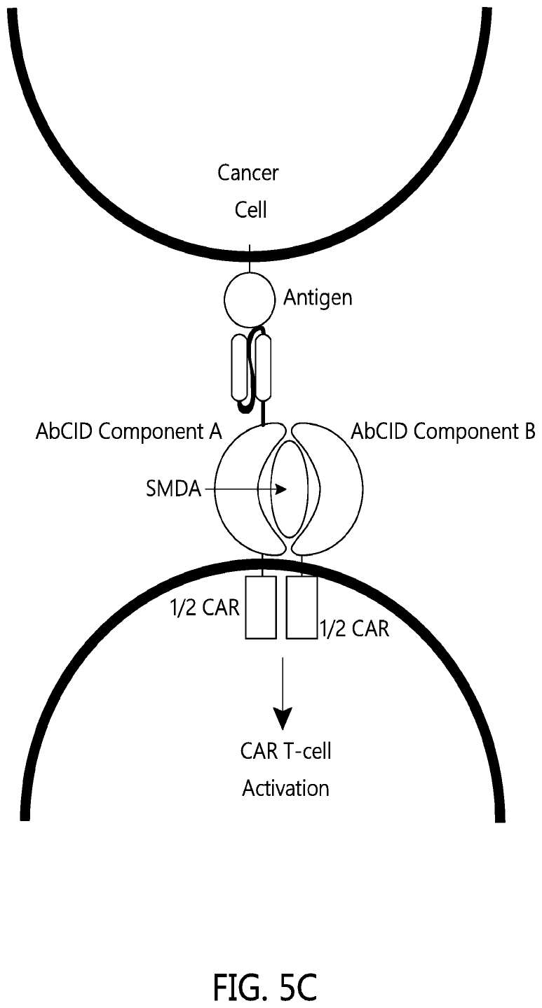

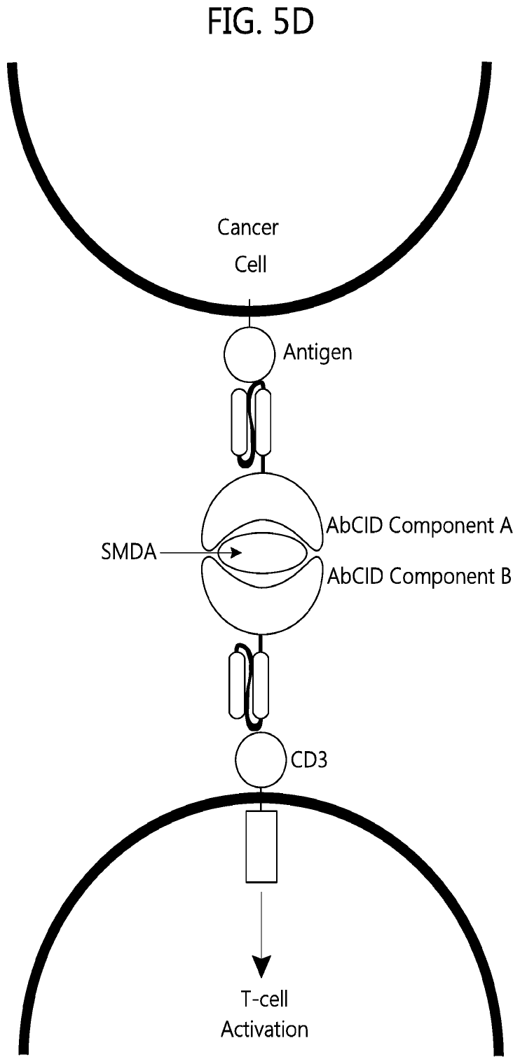

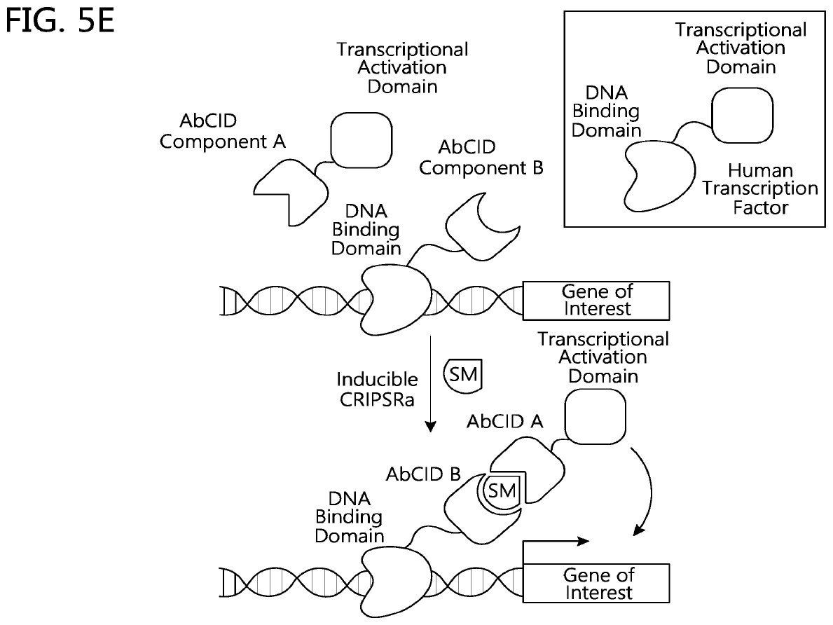

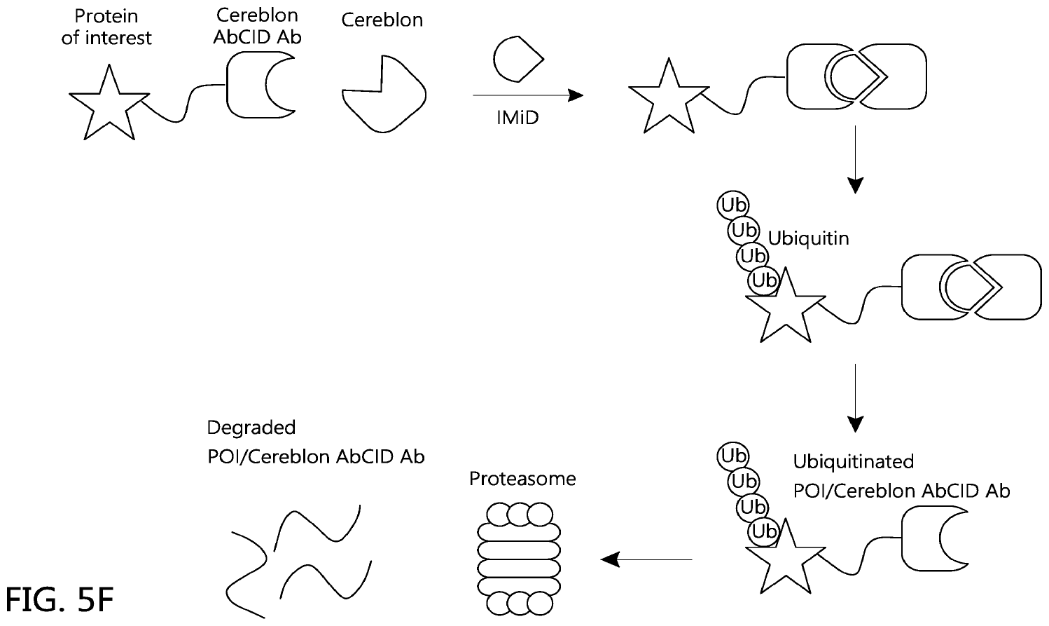

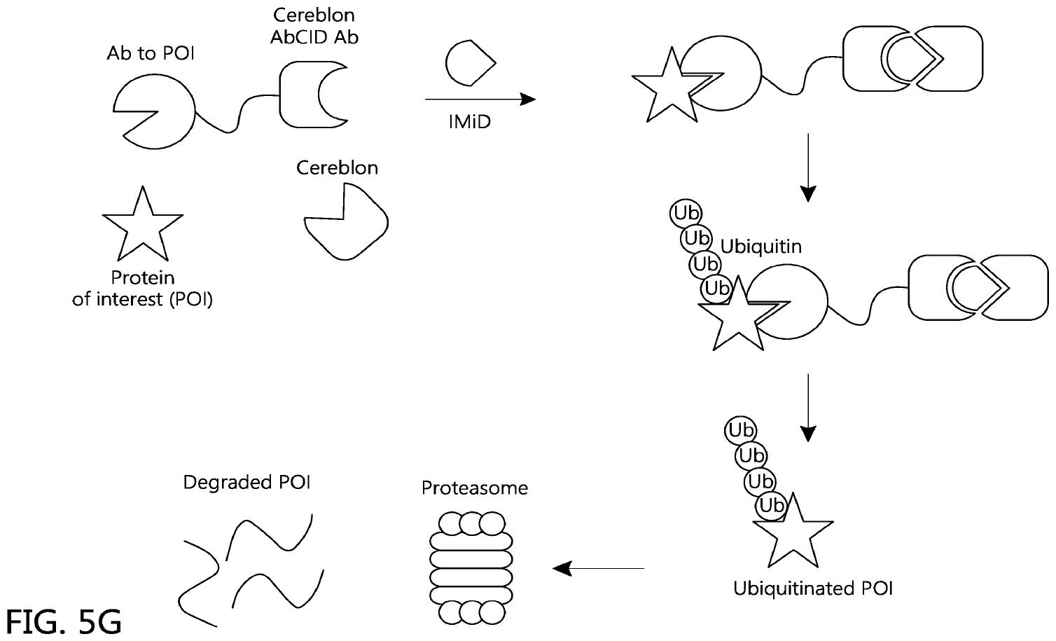

In some embodiments, according to any of the systems described herein, (a) the first adapter moiety comprises a DNA binding domain and the second adapter moiety comprises a transcriptional regulatory domain; or (b) the second adapter moiety comprises a DNA binding domain and the first adapter moiety comprises a transcriptional regulatory domain, wherein the first CID component and the second CID component are configured such that when dimerized in the presence of the IMiD to form the CID, the CID is capable of regulating transcription of a target gene. See FIG. 5E for a schematic showing an example of such a system. In some embodiments, the first CID component further comprises a nuclear localization signal and the second CID component further comprises a nuclear localization signal, In some embodiments, (i) the transcriptional regulatory domain is a transcriptional activation domain, and the CID is capable of upregulating transcription of the target gene; or (ii) the transcriptional regulatory domain is a transcriptional repressor domain, and the CID is capable of downregulating transcription of the target gene. In some embodiments, the transcriptional regulatory domain is a VPR transcriptional activation domain (see, for example, Chavez, et al., Nat. Methods, 12:326-328 (2015)). In some embodiments, the DNA binding domain is derived from a naturally occurring transcriptional regulator. In some embodiments, the DNA binding domain is derived from an RNA-guided endonuclease or a DNA-guided endonuclease. In some embodiments, the RNA-guided endonuclease or DNA-guided endonuclease is catalytically dead. In some embodiments, the DNA binding domain is derived from a catalytically dead Cas9 (dCas9). Examples of adapter moieties, such as dCas9, that can be used in CIDs capable of regulating gene transcription can be found, for example, in U.S. Pat. No. 8,993,233.

In some embodiments, according to any of the systems described herein, (a) the first adapter moiety comprises a DNA binding domain and the second adapter moiety comprises a transcriptional regulatory domain; or (b) the second adapter moiety comprises a DNA binding domain and the first adapter moiety comprises a transcriptional regulatory domain, wherein the first CID component and the second CID component are configured such that when dimerized in the presence of the IMiD to form the CID, the CID is capable of regulating transcription of a target gene, wherein the transcriptional regulatory domain is a VPR transcriptional activation domain and the DNA binding domain is derived from dCas9.

### Kill Switch

In some embodiments, according to any of the systems described herein, the first CID component and the second CID component are configured such that when dimerized in the presence of the IMiD to form a CID associated with a target cell, the CID is capable of inducing target cell death. In some embodiments, the first adapter moiety and the second adapter moiety are together capable of inducing apoptosis in the target cell. In some embodiments, the first adapter moiety and/or the second adapter moiety are derived from a caspase protein. In some embodiments, the target cell is an engineered cell adoptively transferred to an individual. In some embodiments, the target cell is a T cell expressing a chimeric antigen receptor (CAR). Examples of adapter moieties that can be used in CIDs capable of inducing cell death can be found, for example, in U.S. Patent Publication Nos. US20160175359 and US20160166613.

Control of apoptosis by dimerization of proapoptotic proteins with IMiDs should permit an experimenter or clinician to tightly and rapidly control the viability of a cell-based implant that displays unwanted effects. Examples of these effects include, but are not limited to, Graft versus Host (GvH) immune responses against off-target tissue or excessive, uncontrolled growth or metastasis of an implant, or CAR T cell-mediated cytokine release syndrome. Rapid induction of apoptosis will severely attenuate the unwanted cell's function and permit the natural clearance of the dead cells by phagocytic cells, such as macrophages, without undue inflammation. Apoptosis is tightly regulated and naturally uses scaffolds, such as Apaf-1, CRADD/RAIDD, or FADD/Mort1, to oligomerize and activate the caspases that can ultimately kill the cell. Apaf-1 can assemble the apoptotic protease Caspase-9 into a latent complex that then forms an active oligomeric apoptosome upon recruitment of cytochrome C to the scaffold. The key event is oligomerization of the scaffold units causing dimerization and activation of the caspase. Similar adapters, such as CRADD, can oligomerize Caspase-2, leading to apoptosis. The compositions and methods provided herein use, for example, AbCIDs that permit the spontaneous dimerization and activation of caspase units present as adapter moieties upon recruitment with an IMiD.

Using certain of the compositions and methods provided herein, caspase activation occurs only when an IMiD is present to allow dimerization of caspase-fused CID components of an AbCID. In these methods, the two AbCID components must be present as a dimeric unit, not as monomers, to drive caspase dimerization (e.g., caspase-9). The CID components may be localized within the cytosol as soluble entities or present in specific subcellular locales, such as the plasma membrane through targeting signals. The components used to activate apoptosis and the downstream components that degrade the cell are shared by all cells and across species. With regard to Caspase-9 activation, these methods can be broadly utilized in cell lines, in normal primary cells, such as, for example, but not limited to, T cells, or in cell implants. The Caspase-9 polypeptide may be full length or truncated.

Caspase polypeptides other than Caspase-9 that may be used as adapter moieties of the AbCIDs described herein include, for example, Caspase-1, Caspase-3, and Caspase-8. Discussions of these Caspase polypeptides may be found in, for example, MacCorkle, R. A., et al., Proc. Natl. Acad. Sci. U.S.A. (1998) 95:3655-3660; and Fan, L., et al. (1999) Human Gene Therapy 10:2273-2285).

### Chimeric Antigen Receptor (CAR)

In some embodiments, according to any of the systems described herein, the first CID component and the second CID component are configured such that when dimerized in the presence of the IMiD to form a CID associated with a T cell, the CID is a heterodimeric CAR capable of activating the T cell upon binding a target antigen.

In some embodiments, according to any of the systems described herein, (a) the first adapter moiety comprises (i) a transmembrane domain; (ii) a cytoplasmic co-stimulatory domain; and (iii) a cytoplasmic signaling domain; and the second adapter moiety comprises an extracellular antigen-binding moiety; or (b) the second adapter moiety comprises (i) a transmembrane domain; (ii) a cytoplasmic co-stimulatory domain; and (iii) a cytoplasmic signaling domain; and the first adapter moiety comprises an extracellular antigen-binding moiety; wherein the extracellular antigen-binding moiety specifically binds to a target antigen. See, for example, FIG. 5A. In some embodiments, the CID component comprising the extracellular antigen-binding moiety further comprises a secretory signal peptide.

In some embodiments, according to any of the systems described herein, (a) the first adapter moiety comprises (i) a cytoplasmic co-stimulatory domain or a cytoplasmic signaling domain; (ii) a transmembrane domain; and (iii) an extracellular antigen-binding moiety; and the second adapter moiety comprises a cytoplasmic co-stimulatory domain or a cytoplasmic signaling domain; or (b) the second adapter moiety comprises (i) a cytoplasmic co-stimulatory domain or a cytoplasmic signaling domain; (ii) a transmembrane domain; and (iii) an extracellular antigen-binding moiety; and the first adapter moiety comprises a cytoplasmic co-stimulatory domain or a cytoplasmic signaling domain; wherein the extracellular antigen-binding moiety specifically binds to a target antigen. See, for example, FIG. 5B. In some embodiments, the first and second CID components together comprise a cytoplasmic co-stimulatory domain and a cytoplasmic signaling domain.

In some embodiments, according to any of the systems described herein, the first adapter moiety comprises (i) a cytoplasmic co-stimulatory domain or a cytoplasmic signaling domain; and (ii) a transmembrane domain; and the second adapter moiety comprises (i) a cytoplasmic co-stimulatory domain or a cytoplasmic signaling domain; and (ii) a transmembrane domain; wherein the first or second CID component further comprises an extracellular antigen-binding moiety linked to its binding moiety; and wherein the extracellular antigen-binding moiety specifically binds to a target antigen. See, for example, FIG. 5C. In some embodiments, first and second CID components together comprise a cytoplasmic co-stimulatory domain and a cytoplasmic signaling domain.

Examples of adapter moieties that can be used in CIDs to form heterodimeric CARs can be found, for example, in U.S. Pat. No. 9,587,020.

In some embodiments, an AbCID described herein can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell. When present in the plasma membrane of a eukaryotic cell, the AbCID is active in the presence of: 1) the IMiD that allows for dimerization of the first and second CID components; and 2) a factor that binds the extracellular antigen-binding moiety. The factor that binds the extracellular antigen-binding domain can be a soluble (e.g., not bound to a cell) factor; a factor present on the surface of a cell such as a target cell; a factor presented on a solid surface; a factor present in a lipid bilayer; and the like.

In some embodiments, an AbCID of the present disclosure, when present in the plasma membrane of a eukaryotic cell, and when activated by an IMiD, results in cytotoxic activity by the cell toward a target cell that expresses on its cell surface an antigen to which the extracellular antigen-binding domain binds. For example, where the eukaryotic cell is a cytotoxic cell (e.g., an NK cell or a cytotoxic T lymphocyte), an AbCID of the present disclosure, when present in the plasma membrane of the cell, and when activated by an IMiD, increases cytotoxic activity of the cell toward a target cell that expresses on its cell surface an antigen to which the extracellular antigen-binding domain binds. For example, where the eukaryotic cell is an NK cell or a T lymphocyte, an AbCID of the present disclosure, when present in the plasma membrane of the cell, and when activated by an IMiD, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of the IMiD.

In some embodiments, an AbCID of the present disclosure, when present in the plasma membrane of a eukaryotic cell, and when activated by an antigen that binds the extracellular antigen-binding domain and an IMiD, can result in other CAR activation related events such as proliferation, expansion, intracellular signaling modulation, cellular differentiation, or cell death.

An extracellular antigen-binding domain suitable for use in an AbCID of the present disclosure can be any antigen-binding polypeptide, a wide variety of which are known in the art. In some instances, the extracellular antigen-binding domain is a single chain Fv (scFv). Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

An extracellular antigen-binding domain suitable for use in an AbCID of the present disclosure can have a variety of antigen-binding specificities. In some cases, the extracellular antigen-binding domain is specific for an epitope present in an antigen that is expressed by (synthesized by) a cancer cell, i.e., a cancer cell associated antigen. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

### Bispecific T Cell Engager

In some embodiments, according to any of the systems described herein, the first CID component and the second CID component are configured such that when dimerized in the presence of the IMiD to form a CID, the CID is a heterodimeric bispecific T cell engager capable of redirecting a T cell to a target cell. In some embodiments, (a) the first adapter moiety comprises a T cell antigen-binding moiety and the second adapter moiety comprises a target cell antigen-binding moiety; or (b) the second adapter moiety comprises a T cell antigen-binding moiety and the first adapter moiety comprises a target cell antigen-binding moiety. See, e.g., FIG. 5D. In some embodiments, the T cell antigen-binding moiety is an antibody moiety that specifically binds to CD3. In some embodiments, the target cell antigen-binding moiety is an antibody moiety that specifically binds to a cell surface antigen associated with a diseased cell. In some embodiments, the diseased cell is a cancer cell. In some embodiments, the target cell antigen-binding moiety is an antibody moiety that specifically binds to CD19. Examples of adapter moieties that can be used in CIDs to form heterodimeric bispecific T cell engagers can be found, for example, in U.S. Patent Publication No. US20140050660.

### T Cell Modulation

In some embodiments, according to any of the systems described herein, the first CID component and the second CID component are configured such that when dimerized in the presence of the IMiD to form a CID associated with an immune cell, the CID is a heterodimeric signaling molecule capable of modulating activation of the immune cell. In some embodiments, the first adapter moiety comprises (i) a transmembrane domain; and (ii) a cytoplasmic co-stimulatory domain; and the second adapter moiety comprises (i) a transmembrane domain; and (ii) a cytoplasmic co-stimulatory domain. In some embodiments, the immune cell is a T cell. In some embodiments, the T cell is a CAR T cell. Examples of adapter moieties that can be used in CIDs to form heterodimeric signaling molecules capable of modulating activation of an immune cell can be found, for example, in U.S. Patent Publication No. 20140286987.

In some embodiments, the adapter moieties of an AbCID described herein comprise one or more co-stimulatory polypeptides, such as, for example, CD28 and 4-1BB, with and without the CD3 zeta chain, to enable AbCID-dependent proliferation and co-stimulation. The AbCID may be used alone to provide co-stimulation, and increase a T cell immune response. Using such AbCIDs, a population of T cells, for example a population with non-specific targets, may be transfected or transformed with DNA encoding an AbCID, then administered to a subject to enhance a general immune response. These AbCIDs may also be expressed in a cell along with a CAR. In such methods, an inducible AbCID is used in combination with a CAR, thereby segregating CAR signaling into two separate functions. This second function, provided by the CAR, provides antigen-specific cytotoxicity to the engineered T cells.

Co-stimulatory polypeptide molecules are capable of amplifying the cell-mediated immune response through activation of signaling pathways involved in cell survival and proliferation. Co-stimulatory proteins that are contemplated include, for example, the members of tumor necrosis factor receptor (TNFR) family (i.e., CD40, RANK/TRANCE-R, OX40, and 4-1BB) and CD28 family members (CD28, ICOS). Co-stimulatory proteins may include, for example, CD28, 4-1BB, and OX40. Stimulatory proteins may include, for example, the CD3 zeta chain. More than one co-stimulatory polypeptide, or co-stimulatory polypeptide cytoplasmic region may be used in the inducible AbCIDs described herein. For example, the AbCID may comprise a CD28 cytoplasmic polypeptide and a 4-1BB cytoplasmic polypeptide. Or, for example, the AbCID may comprise a CD28 cytoplasmic polypeptide and an OX40 cytoplasmic polypeptide. Or, for example, the AbCID may further comprise a CD3 zeta domain polypeptide.

### Protein Degradation

In some embodiments, according to any of the systems described herein, the first CID component and the second CID component are configured such that when dimerized in the presence of the IMiD to form a CID in a cell, association of the second CID component with the first CID component allows for ubiquitination of the second CID component. In some embodiments, dimerization of the CID results in ubiquitination of the second CID component such that it is targeted for degradation. In some embodiments, the first CID component is endogenous cereblon in the cell. In some embodiments, the second CID component is part of a fusion protein comprising the second CID component fused to a protein-of-interest (POI). See, e.g., FIG. 5F. Exemplary POIs include, without limitation, therapeutic proteins, CARs, and cytokines (such as IL-2, IL-12, and IL-15).

Thus, in some embodiments, provided herein is a system comprising (a) a first CID component comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a POI fused to a second binding moiety that specifically binds to the complex between the first CID component and the IMiD, or a second nucleic acid encoding polypeptide components of the second CID component, wherein the first CID component and the second CID component are configured such that when dimerized in the presence of the IMiD to form a CID in a cell, association of the second CID component with the first CID component allows for ubiquitination of the second CID component. In some embodiments, the IMiD is thalidomide. In some embodiments, the IMiD is lenalidomide. In some embodiments, the IMiD is pomalidomide. In some embodiments, dimerization of the CID results in ubiquitination of the second CID component such that it is targeted for degradation. In some embodiments, the first CID component is endogenous cereblon in the cell. In some embodiments, the POI is a therapeutic protein. In some embodiments, the POI is a CAR. In some embodiments, the POI is a cytokine, such as IL-2, IL-12, or IL-15. In some embodiments, the second binding moiety is an antibody moiety comprising a heavy chain variable domain and a light chain variable domain comprising HC-CDRs and LC-CDRs from a Fab-phage clone as shown in Table 1, or variants thereof having at least 85% identity.

In some embodiments, according to any of the systems described herein, the first CID component and the second CID component are configured such that when dimerized in the presence of the IMiD to form a CID in a cell, association of the second CID component with the first CID component allows for ubiquitination of a POI bound to the second CID component. In some embodiments, dimerization of the CID results in ubiquitination of the POI such that it is targeted for degradation. In some embodiments, the first CID component is endogenous cereblon in the cell. In some embodiments, the second CID component comprises a second adapter moiety comprising a POI-binding moiety capable of binding the POI. In some embodiments, the POI-binding moiety is an antibody moiety specific for the POI. In some embodiments, the POI-binding moiety is a naturally occurring cognate binding partner of the POI or a POI-binding fragment or derivative thereof. See, e.g., FIG. 5F. Exemplary POIs include, without limitation, pathogen-associated proteins, aberrantly expressed endogenous proteins, CARs, and cytokines (such as IL-2, IL-12, and IL-15).

Thus, in some embodiments, provided herein is a system comprising (a) a first CID component comprising cereblon or an IMiD-binding fragment or derivative thereof, wherein the first binding moiety is capable of interacting with an IMiD (e.g., thalidomide, lenalidomide, or thalidomide) to form a complex between the first CID component and the IMiD, or a first nucleic acid encoding polypeptide components of the first CID component; and (b) a second CID component comprising a POI-binding moiety fused to a second binding moiety that specifically binds to the complex between the first CID component and the IMiD, or a second nucleic acid encoding polypeptide components of the second CID component, wherein the first CID component and the second CID component are configured such that when dimerized in the presence of the IMiD to form a CID in a cell, association of the second CID component with the first CID component allows for ubiquitination of a POI bound to the POI-binding moiety. In some embodiments, the IMiD is thalidomide. In some embodiments, the IMiD is lenalidomide. In some embodiments, the IMiD is pomalidomide. In some embodiments, dimerization of the CID results in ubiquitination of the POI such that it is targeted for degradation. In some embodiments, the first CID component is endogenous cereblon in the cell. In some embodiments, the POI-binding moiety is an antibody moiety specific for the POI. In some embodiments, the POI-binding moiety is a naturally occurring cognate binding partner of the POI or a POI-binding fragment or derivative thereof. In some embodiments, the POI is a pathogen-associated protein. In some embodiments, the POI is an aberrantly expressed endogenous protein. In some embodiments, the POI is a CAR. In some embodiments, the POI is a cytokine, such as IL-2, IL-12, or IL-15. In some embodiments, the second binding moiety is an antibody moiety comprising a heavy chain variable domain and a light chain variable domain comprising HC-CDRs and LC-CDRs from a Fab-phage clone as shown in Table 1, or variants thereof having at least 85% identity.

In some embodiments, a second CID component according to any of the embodiments described in this section, when present in a cell expressing cereblon or an IMiD-binding fragment or derivative thereof capable of facilitating ubiquitination, and when activated by an IMiD, allows for degradation of a POI bound or fused to the second CID component. In some embodiments, the level of the POI in the cell is decreased by at least about 10% (such as at least about any of 20%, 30%, 40%, 50%, 60%, 70%, 80%, 90%, 95%, or greater) compared to the level of the POI in the cell prior to modifying the amount of the IMiD in the cell.

### Methods of Producing an AbCID

In some embodiments, provided herein is a method of selecting binding moieties from a binding molecule library, wherein the binding moieties specifically bind to a complex between an IMiD and an IMiD-binding moiety comprising cereblon or an IMiD-binding fragment or derivative thereof, comprising: (a) screening an input set of binding moieties for binding moieties that do not bind to the IMiD-binding moiety in the absence of the IMiD, thereby generating a set of counter selected binding moieties; and (b) screening an input set of binding moieties for binding moieties that bind to the complex of the IMiD and the IMiD-binding moiety, thereby generating a set of positively selected binding moieties; and (c) conducting one or more rounds of screening, wherein each round of screening comprises the screening of step (a) and the screening of step (b), such that a set of binding moieties that specifically bind to the complex between the IMiD and the IMiD-binding moiety is generated. In some embodiments, the method comprises two or more rounds of screening, wherein (1) the input set of binding moieties of step (a) for the first round of screening is the binding molecule library, (2) the input set of binding moieties of step (b) for each round of screening is the set of counter selected binding moieties of step (a) from the given round of screening, (3) the input set of binding moieties of step (a) for each round of screening following the first round of screening is the set of positively selected binding moieties of step (b) from the previous round of screening, and (4) the set of binding moieties that specifically bind to the complex between the IMiD and the IMiD-binding moiety is the set of positively selected binding moieties of step (b) for the last round of screening. In some embodiments, the method comprises at least 2 (such as at least any of 2, 3, 4, 5, 6, or more) rounds of selection. In some embodiments, at least one of the binding moieties in the set of binding moieties that specifically bind to the complex between the IMiD and the IMiD-binding moiety binds to the complex with a dissociation constant (K_{d}) no more than about 1/250 times (such as no more than about any of 1/300, 1/350, 1/400, 1/450, 1/500, 1/600, 1/700, 1/800, 1/900, 1/1000, 1/1100, 1/1200, 1/1300, 1/1400, or 1/1500 times, or less) its K_{d} for binding to each of the free IMiD and the free IMiD-binding moiety. In some embodiments, each of the binding moieties in the set of binding moieties that specifically bind to the complex between the IMiD and the IMiD-binding moiety binds to the complex with a dissociation constant (K_{d}) no more than about 1/250 times (such as no more than about any of 1/300, 1/350, 1/400, 1/450, 1/500, 1/600, 1/700, 1/800, 1/900, 1/1000, 1/1100, 1/1200, 1/1300, 1/1400, or 1/1500 times, or less) its K_{d} for binding to each of the free IMiD and the free IMiD-binding moiety. In some embodiments, the binding molecule library is an antibody library, a DARPin library, a nanobody library, or an aptamer library. In some embodiments, the binding molecule library is an antibody library. In some embodiments, the antibody library is a phage-displayed Fab library.

In some embodiments, provided herein is a construct comprising an antibody moiety that specifically binds to a complex between an IMiD and an IMiD-binding moiety prepared by a process comprising the steps of: (A) selecting antibody moieties that specifically bind to the complex between the IMiD and the IMiD-binding moiety from an antibody library according to any of the methods described herein; and (B) providing a construct comprising one of the antibodies moieties of (A). In some embodiments, the construct is a second CID component of an AbCID according to any of the embodiments described herein, and the first CID component of the AbCID comprises the IMiD-binding moiety. In some embodiments, the antibody moiety specifically binds to a site of the complex comprising at least a portion of the IMiD and a portion of the IMiD-binding moiety. In some embodiments, the site of the complex comprising at least a portion of the IMiD and a portion of the IMiD-binding moiety is an interface between the IMiD and a binding site of the IMiD-binding moiety for the IMiD, comprising at least one atom of the IMiD and one atom of the IMiD-binding moiety.

In some embodiments, provided herein is a system according to any of the embodiments described herein, wherein the second binding moiety is an antibody moiety selected by a process comprising the steps of: (A) selecting antibody moieties that specifically bind to the complex between the IMiD and the first binding moiety from an antibody library according to any of the methods described herein; and (B) selecting the second binding moiety to be one of the antibodies moieties of (A). In some embodiments, the antibody moiety specifically binds to a site of the complex comprising at least a portion of the IMiD and a portion of the first binding moiety. In some embodiments, the site of the complex comprising at least a portion of the IMiD and a portion of the first binding moiety is an interface between the IMiD and a binding site of the first binding moiety for the IMiD, comprising at least one atom of the IMiD and one atom of the first binding moiety.

### Methods of Using an AbCID

### Transcriptional Regulation

In some embodiments, provided herein is a method of modulating the expression of a target gene in a cell, comprising expressing in the cell the first and second CID components of a system according to any of the embodiments described herein wherein the CID is capable of regulating transcription of a target gene, and modifying the amount of the IMiD in the cell to modulate the expression of the target gene.

In some embodiments, provided herein is a method of modulating the expression of a target gene in a cell, comprising: (A) expressing in the cell (a) a first CID component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises a DNA binding domain and the second adapter moiety comprises a transcriptional regulatory domain; or (2) the second adapter moiety comprises a DNA binding domain and the first adapter moiety comprises a transcriptional regulatory domain, wherein the first CID component and the second CID component are configured such that when dimerized in the presence of the IMiD to form the CID, the CID is capable of regulating transcription of the target gene; and (B) modifying the amount of the IMiD in the cell to modulate the expression of the target gene. In some embodiments, the first CID component further comprises a nuclear localization signal and the second CID component further comprises a nuclear localization signal. In some embodiments, (i) the transcriptional regulatory domain is a transcriptional activation domain, and the CID is capable of upregulating transcription of the target gene; or (ii) the transcriptional regulatory domain is a transcriptional repressor domain, and the CID is capable of downregulating transcription of the target gene. In some embodiments, the transcriptional regulatory domain is a VPR transcriptional activation domain. In some embodiments, the DNA binding domain is derived from a naturally occurring transcriptional regulator. In some embodiments, the DNA binding domain is derived from an RNA-guided endonuclease or a DNA-guided endonuclease. In some embodiments, the RNA-guided endonuclease or DNA-guided endonuclease is catalytically dead. In some embodiments, the DNA binding domain is derived from a catalytically dead Cas9 (dCas9). In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

### Cell Survival

In some embodiments, provided herein is a method of controlling the survival of target cells in an individual, comprising: (A) expressing in the target cells the first and second CID components of a system according to any of the embodiments described herein wherein the CID is capable of inducing target cell death; and (B) administering to the individual the IMiD in a regimen effective to (I) kill a predetermined amount of the target cells; or (II) maintain a predetermined amount of the target cells.

In some embodiments, provided herein is a method of controlling the survival of target cells in an individual, comprising: (A) expressing in the target cells (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the first adapter moiety and the second adapter moiety are together capable of inducing apoptosis in the target cell; and (B) administering to the individual the IMiD in a regimen effective to (I) kill a predetermined amount of the target cells; or (II) maintain a predetermined amount of the target cells. In some embodiments, the first adapter moiety and/or the second adapter moiety are derived from a caspase protein. In some embodiments, the target cells are engineered cells adoptively transferred to the individual. In some embodiments, the target cells are part of an adoptive cell therapy in the individual. In some embodiments, the target cells are T cells expressing a chimeric antigen receptor (CAR). In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

### Immune Modulation

In some embodiments, provided herein is a method of modulating an immune response to a target cell in an individual, comprising: (A) administering to the individual modified T cells expressing the first and second CID components of a system according to any of the embodiments described herein wherein the CID is a heterodimeric CAR specific for a target antigen, and wherein the target antigen is expressed on the surface of the target cell; and (B) administering to the individual the IMiD in a regimen effective to modulate an immune response to the target cell.

In some embodiments, provided herein is a method of modulating an immune response to a target cell in an individual, comprising: (A) administering to the individual modified T cells expressing (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises (i) a transmembrane domain; (ii) a cytoplasmic co-stimulatory domain; and (iii) a cytoplasmic signaling domain; and the second adapter moiety comprises an extracellular antigen-binding moiety; or (2) the second adapter moiety comprises (i) a transmembrane domain; (ii) a cytoplasmic co-stimulatory domain; and (iii) a cytoplasmic signaling domain; and the first adapter moiety comprises an extracellular antigen-binding moiety; wherein the extracellular antigen-binding moiety specifically binds to the target antigen; and (B) administering to the individual the IMiD in a regimen effective to modulate an immune response to the target cell. In some embodiments, the CID component comprising the extracellular antigen-binding moiety further comprises a secretory signal peptide. In some embodiments, the regimen is effective to maintain an immune response to the target cell with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of modulating an immune response to a target cell in an individual, comprising: (A) administering to the individual modified T cells expressing a first chemically induced dimer (CID) component comprising (i) a first binding moiety comprising cereblon or an IMiD-binding fragment or derivative thereof; and (ii) a first adapter moiety linked to the first binding moiety, wherein the first adapter moiety comprises (i) a transmembrane domain; (ii) a cytoplasmic co-stimulatory domain; and (iii) a cytoplasmic signaling domain; (B) administering to the individual a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the second adapter moiety comprises an extracellular antigen-binding moiety, and wherein the extracellular antigen-binding moiety specifically binds to the target antigen; and (C) administering to the individual the IMiD in a regimen effective to modulate an immune response to the target cell. In some embodiments, the regimen is effective to maintain an immune response to the target cell with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of modulating an immune response to a target cell in an individual, comprising: (A) administering to the individual a first chemically induced dimer (CID) component comprising (i) a first binding moiety comprising cereblon or an IMiD-binding fragment or derivative thereof; and (ii) a first adapter moiety linked to the first binding moiety, wherein the first adapter moiety comprises an extracellular antigen-binding moiety, and wherein the extracellular antigen-binding moiety specifically binds to the target antigen; (B) administering to the individual modified T cells expressing a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the second adapter moiety comprises (i) a transmembrane domain; (ii) a cytoplasmic co-stimulatory domain; and (iii) a cytoplasmic signaling domain; and (C) administering to the individual the IMiD in a regimen effective to modulate an immune response to the target cell. In some embodiments, the regimen is effective to maintain an immune response to the target cell with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of modulating an immune response to a target cell in an individual, comprising: (A) administering to the individual the first and second CID components of a system according to any of the embodiments described herein wherein the CID is a heterodimeric bispecific T cell engager capable of redirecting a T cell to the target cell; and (B) administering to the individual the IMiD in a regimen effective to modulate an immune response to the target cell.

In some embodiments, provided herein is a method of modulating an immune response to a target cell in an individual, comprising: (A) administering to the individual (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises a T cell antigen-binding moiety and the second adapter moiety comprises a target cell antigen-binding moiety; or (2) the second adapter moiety comprises a T cell antigen-binding moiety and the first adapter moiety comprises a target cell antigen-binding moiety; and (B) administering to the individual the IMiD in a regimen effective to modulate an immune response to the target cell. In some embodiments, the T cell antigen-binding moiety is an antibody moiety that specifically binds to CD3. In some embodiments, the target cell antigen-binding moiety is an antibody moiety that specifically binds to a cell surface antigen associated with a diseased cell. In some embodiments, the diseased cell is a cancer cell. In some embodiments, the target cell antigen-binding moiety is an antibody moiety that specifically binds to CD19. In some embodiments, the regimen is effective to maintain an immune response to the target cell with fewer adverse effects in the individual as compared to a corresponding method comprising administration of a conventional bispecific T cell engager (e.g., BiTE®) comprising the corresponding bispecific T cell engager domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of modulating an immune response mediated by T cells in an individual, comprising: (A) expressing in the T cells the first and second CID components of a system according to any of the embodiments described herein wherein the CID is a heterodimeric signaling molecule capable of modulating activation of the immune cell; and (B) administering to the individual the IMiD in a regimen effective to modulate an immune response mediated by the T cells.

In some embodiments, provided herein is a method of modulating an immune response mediated by T cells in an individual, comprising: (A) expressing in the T cells (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the first adapter moiety comprises (i) a transmembrane domain; and (ii) a cytoplasmic co-stimulatory domain; and the second adapter moiety comprises (i) a transmembrane domain; and (ii) a cytoplasmic co-stimulatory domain; and (B) administering to the individual the IMiD in a regimen effective to modulate an immune response mediated by the T cells. In some embodiments, the regimen is effective to maintain an immune response mediated by the T cells with fewer adverse effects in the individual as compared to a corresponding method comprising expression of a monomeric signaling molecule comprising the corresponding signaling domains of the CID in the T cells. In some embodiments, the T cells are CAR T cells. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of modulating an immune response mediated by T cells in an individual, comprising: (A) expressing in the T cells the second CID component of a system according to any of the embodiments described herein wherein dimerization of the first and second CID components in the presence of an IMiD allows for ubiquitination of i) a CAR or cytokine (e.g., IL-2, IL-12, or IL-15) associated with the second CID component or ii) a fusion protein comprising the CAR or cytokine and the second CID component, and the ubiquitination of the CAR or cytokine or fusion protein thereof targets it for degradation; and (B) administering to the individual the IMiD in a regimen effective to modulate an immune response mediated by the T cells.

In some embodiments, provided herein is a method of modulating an immune response mediated by T cells in an individual, comprising: (A) expressing in the T cells a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a CAR, wherein the T cells are CAR T cells comprising a first CID component comprising cereblon or an IMiD-binding fragment or derivative thereof capable of facilitating ubiquitination; and (B) administering to the individual the IMiD in a regimen effective to modulate an immune response mediated by the T cells. In some embodiments, dimerization of the CID results in ubiquitination of the second CID component comprising the CAR such that it is targeted for degradation. In some embodiments, the first CID component is endogenous cereblon in the T cells. In some embodiments, the regimen is effective to maintain an immune response mediated by the T cells with fewer adverse effects in the individual as compared to an immune response mediated by the T cells in the absence of the IMiD. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of modulating an immune response mediated by T cells in an individual, comprising: (A) expressing in the T cells a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a cytokine (e.g., IL-2, IL-12, or IL-15), wherein the T cells comprise a first CID component comprising cereblon or an IMiD-binding fragment or derivative thereof capable of facilitating ubiquitination; and (B) administering to the individual the IMiD in a regimen effective to modulate an immune response mediated by the T cells. In some embodiments, the T cells are CAR T cells. In some embodiments, dimerization of the CID results in ubiquitination of the second CID component comprising the cytokine such that it is targeted for degradation. In some embodiments, the first CID component is endogenous cereblon in the T cells. In some embodiments, the regimen is effective to maintain an immune response mediated by the T cells with fewer adverse effects in the individual as compared to an immune response mediated by the T cells in the absence of the IMiD. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of modulating an immune response mediated by CAR T cells comprising a CAR in an individual, comprising: (A) expressing in the CAR T cells a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a CAR-binding moiety capable of binding to the CAR present in the CAR T cells, wherein the CAR T cells comprise a first CID component comprising cereblon or an IMiD-binding fragment or derivative thereof capable of facilitating ubiquitination; and (B) administering to the individual the IMiD in a regimen effective to modulate an immune response mediated by the CAR T cells. In some embodiments, dimerization of the CID results in ubiquitination of a CAR bound to the CAR-binding moiety such that the CAR is targeted for degradation. In some embodiments, the first CID component is endogenous cereblon in the CAR T cells. In some embodiments, the regimen is effective to maintain an immune response mediated by the CAR T cells with fewer adverse effects in the individual as compared to an immune response mediated by the CAR T cells in the absence of the IMiD. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of modulating an immune response mediated by T cells in an individual, comprising: (A) expressing in the T cells a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a cytokine-binding moiety capable of binding to a cytokine (e.g., IL-2, IL-12, or IL-15) present in the T cells, wherein the T cells comprise a first CID component comprising cereblon or an IMiD-binding fragment or derivative thereof capable of facilitating ubiquitination; and (B) administering to the individual the IMiD in a regimen effective to modulate an immune response mediated by the T cells. In some embodiments, the T cells are CAR T cells. In some embodiments, dimerization of the CID results in ubiquitination of a cytokine bound to the cytokine-binding domain such that it is targeted for degradation. In some embodiments, the first CID component is endogenous cereblon in the T cells. In some embodiments, the regimen is effective to maintain an immune response mediated by the T cells with fewer adverse effects in the individual as compared to an immune response mediated by the T cells in the absence of the IMiD. In some embodiments, the IMiD is thalidomide. In some embodiments, the IMiD is lenalidomide. In some embodiments, the IMiD is pomalidomide. In some embodiments, the cytokine is IL-2, IL-12, or IL-15. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

### Protein Degradation

In some embodiments, provided herein is a method of modulating the level of a POI in a cell, comprising a) expressing in the cell the second CID component of a system according to any of the embodiments described herein wherein dimerization of the first and second CID components allows for ubiquitination of i) a POI associated with the second CID component or ii) a fusion protein comprising the POI and the second CID component, and the ubiquitination of the POI or fusion protein targets it for degradation, and b) modifying the amount of the IMiD in the cell to modulate the level of the POI in the cell.

In some embodiments, provided herein is a method of modulating the level of a POI in a cell, comprising a) expressing in the cell a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises the POI, wherein the cell comprises a first CID component comprising cereblon or an IMiD-binding fragment or derivative thereof; and b) modifying the amount of the IMiD in the cell to modulate the level of the POI in the cell. In some embodiments, the IMiD is thalidomide. In some embodiments, the IMiD is lenalidomide. In some embodiments, the IMiD is pomalidomide. In some embodiments, dimerization of the CID results in ubiquitination of the second CID component such that it is targeted for degradation. In some embodiments, the first CID component is endogenous cereblon in the cell. In some embodiments, the POI is a therapeutic protein. In some embodiments, the POI is a CAR. In some embodiments, the POI is a cytokine, such as IL-2, IL-12, or IL-15. In some embodiments, the second binding moiety is an antibody moiety comprising a heavy chain variable domain and a light chain variable domain comprising HC-CDRs and LC-CDRs from a Fab-phage clone as shown in Table 1, or variants thereof having at least 85% identity.

In some embodiments, provided herein is a method of modulating the level of a POI in a cell, comprising a) expressing in the cell a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a POI-binding moiety, wherein the cell comprises a first CID component comprising cereblon or an IMiD-binding fragment or derivative thereof; and b) modifying the amount of the IMiD in the cell to modulate the level of the POI in the cell. In some embodiments, the IMiD is thalidomide. In some embodiments, the IMiD is lenalidomide. In some embodiments, the IMiD is pomalidomide. In some embodiments, dimerization of the CID results in ubiquitination of POI bound to the POI-binding moiety such that it is targeted for degradation. In some embodiments, the first CID component is endogenous cereblon in the cell. In some embodiments, the POI-binding moiety is an antibody moiety specific for the POI. In some embodiments, the POI-binding moiety is a naturally occurring cognate binding partner of the POI or a POI-binding fragment or derivative thereof. In some embodiments, the POI is a pathogen-associated protein. In some embodiments, the POI is an aberrantly expressed endogenous protein. In some embodiments, the POI is a CAR. In some embodiments, the POI is a cytokine, such as IL-2, IL-12, or IL-15. In some embodiments, the second binding moiety is an antibody moiety comprising a heavy chain variable domain and a light chain variable domain comprising HC-CDRs and LC-CDRs from a Fab-phage clone as shown in Table 1, or variants thereof having at least 85% identity.

In some embodiments, according to any of the methods of modulating the level of a POI in a cell described herein, the amount of the IMiD in the cell is modified such that the level of the POI in the cell is decreased by at least about 10% (such as at least about any of 20%, 30%, 40%, 50%, 60%, 70%, 80%, 90%, 95%, or greater) compared to the level of the POI in the cell prior to modifying the amount of the IMiD in the cell.

### Cells

In some aspects, provided herein are engineered cells, such as engineered mammalian cells (e.g., T cells), comprising one or more components of an AbCID as set forth and described herein. In some embodiments, the AbCID comprises a first CID component and a second CID component. In some embodiments, the engineered cells comprises the first CID component and/or nucleic acid encoding the first CID component. In some embodiments, the engineered cells comprises the second CID component and/or nucleic acid encoding the second CID component. In some embodiments, the engineered cells comprises i) the first CID component and/or nucleic acid encoding the first CID component and ii) the second CID component and/or nucleic acid encoding the second CID component. In some embodiments, the engineered cells are engineered T cells. In some embodiments, the engineered T cells are human.

In some embodiments, an engineered cell described herein comprises a first CID component of an AbCID. In some embodiments, the first CID component comprises (i) a first binding moiety comprising cereblon or an IMiD-binding fragment or derivative thereof; and (ii) a first adapter moiety linked to the first binding moiety. In some embodiments, the engineered cell further comprises a second CID component of the AbCID. In some embodiments, the second CID component comprises (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety. In some embodiments, the engineered cell does not comprise a second CID component of the AbCID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, an engineered cell described herein comprises a second CID component of an AbCID. In some embodiments, the second CID component comprises (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety. In some embodiments, the engineered cell further comprises a first CID component of the AbCID. In some embodiments, the first CID component comprises (i) a first binding moiety comprising cereblon or an IMiD-binding fragment or derivative thereof; and (ii) a first adapter moiety linked to the first binding moiety. In some embodiments, the engineered cell does not comprise a first CID component of the AbCID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, an engineered cell described herein comprises nucleic acid encoding a first CID component of an AbCID. In some embodiments, the first CID component comprises (i) a first binding moiety comprising cereblon or an IMiD-binding fragment or derivative thereof; and (ii) a first adapter moiety linked to the first binding moiety. In some embodiments, the engineered cell further comprises the first CID component. In some embodiments, the engineered cell further comprises nucleic acid encoding a second CID component of the AbCID. In some embodiments, the second CID component comprises (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety. In some embodiments, the engineered cell further comprises the second CID component. In some embodiments, the engineered cell does not comprise nucleic acid encoding a second CID component of the AbCID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, an engineered cell described herein comprises nucleic acid encoding a second CID component of an AbCID. In some embodiments, the second CID component comprises (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety. In some embodiments, the engineered cell further comprises the second CID component. In some embodiments, the engineered cell further comprises nucleic acid encoding a first CID component of the AbCID. In some embodiments, the first CID component comprises (i) a first binding moiety comprising cereblon or an IMiD-binding fragment or derivative thereof; and (ii) a first adapter moiety linked to the first binding moiety. In some embodiments, the engineered cell further comprises the first CID component. In some embodiments, the engineered cell does not comprise nucleic acid encoding a first CID component of the AbCID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, the engineered cells are T cells, or precursor cells capable of differentiating into T cells. In some embodiments, the engineered cells are CD3+, CD8+, and/or CD4+T lymphocytes. In some embodiments, the engineered cells are CD8+T cytotoxic lymphocyte cells, which may include naïve CD8+ T cells, central memory CD8+ T cells, effector memory CD8+ T cells, or bulk CD8+ T cells.

The lymphocytes (T lymphocytes) can be collected in accordance with known techniques and enriched or depleted by known techniques such as affinity binding to antibodies such as flow cytometry and/or immunomagnetic selection. After enrichment and/or depletion steps, in vitro expansion of the desired T lymphocytes can be carried out in accordance with known techniques or variations thereof that will be apparent to those skilled in the art. In some embodiments, the T cells are autologous T cells obtained from a patient.

For example, the desired T cell population or subpopulation can be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). The non-dividing feeder cells can comprise gamma-irradiated PBMC feeder cells. In some embodiments, the PBMC are irradiated with gamma rays in the range of 3000 to 3600 rads to prevent cell division. In some embodiments, the PBMC are irradiated with gamma rays of 3000, 3100, 3200, 3300, 3400, 3500 or 3600 rads or any value of rads between any two endpoints of any of the listed values to prevent cell division. The order of addition of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least 25° C., preferably at least 30° C., more preferably 37° C. In some embodiments, the temperature for the growth of human T lymphocytes is 22, 24, 26, 28, 30, 32, 34, 36, 37° C., or any other temperature between any two endpoints of any of the listed values.

After isolation of T lymphocytes both cytotoxic and helper T lymphocytes can be sorted into naïve, memory, and effector T cell subpopulations either before or after expansion.

CD8+ cells can be obtained by using standard methods. In some embodiments, CD8+ cells are further sorted into naïve, central memory, and effector memory cells by identifying cell surface antigens that are associated with each of those types of CD8+ cells. In some embodiments, memory T cells are present in both CD62L+ and CD62L− subsets of CD8+ peripheral blood lymphocytes. PBMC are sorted into CD62L-CD8+ and CD62L+CD8+ fractions after staining with anti-CD8 and anti-CD62L antibodies. In some embodiments, the expression of phenotypic markers of central memory T_{CM} include CD45RO, CD62L, CCR7, CD28, CD3, and/or CD127 and are negative or low for granzyme B. In some embodiments, central memory T cells are CD45RO+, CD62L+, and/or CD8+ T cells. In some embodiments, effector TE are negative for CD62L, CCR7, CD28, and/or CD127, and positive for granzyme B and/or perforin. In some embodiments, naïve CD8+T lymphocytes are characterized by the expression of phenotypic markers of naïve T cells comprising CD62L, CCR7, CD28, CD3, CD127, and/or CD45RA.

### Chimeric and Humanized Antibodies

In some embodiments, the antibodies of the invention are derived from a mixture from different species, e.g. a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference.

In certain embodiments, the antibodies of the invention comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### Methods of Treatment

Exemplary methods of the invention are directed to the use of AbCIDs to treat subjects having a disease or condition. Exemplary diseases include, without limitation, cancer, invasive angiogenesis, and autoimmune diseases.

In one embodiment, treatment of a subject in need thereof includes the application or administration of an AbCID of the invention to an isolated tissue, cells or cell line from a patient, where the patient has a disease, a symptom of a disease, or a predisposition toward a disease, e.g., a T cell or CAR T cell. In another embodiment, treatment is also intended to include the application or administration of a pharmaceutical composition comprising the AbCID of the invention to an isolated tissue, cells or cell line from a patient, who has a disease, a symptom of a disease, or a predisposition toward a disease. Exemplary pharmaceutical compositions include an AbCID or a protein complex thereof in admixture with a pharmaceutically acceptable carrier, excipient, etc.

Exemplary AbCIDs of the present invention are useful for the treatment of various malignant and non-malignant tumors. By “anti-tumor activity” is intended a reduction in the rate of malignant cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. For example, therapy with at least one AbCID causes a physiological response, for example, a reduction in angiogenesis, that is beneficial with respect to treatment of disease states in a human.

In one embodiment, the invention relates to AbCID molecules, e.g., antibodies or binding fragments thereof and conjugates thereof, according to the present invention for use as a medicament, in particular for use in the treatment or prophylaxis of cancer or for use in a precancerous condition or lesion. In certain embodiments, the AbCID of the invention is used for the treatment of lymphoma or leukemia.

Further AbCIDs of the present invention can also be used to inhibit angiogenesis for the treatment of pathological conditions dependent upon the formation of new blood vessels, including tumor development and macular degeneration. Angiogenesis is a complex multistep morphogenetic event during which endothelial cells, stimulated by major determinants of vascular remodeling, dynamically modify their cell-to-cell and cell-to-matrix contacts and move directionally to be reorganized into a mature vascular tree (Bussolino et al., Trends Biochem Sci. 22:251-256 (1997); Risau, Nature 386:671-674 (1997); Jain, Nat. Med. 9:685-693 (2003)). The formation of new blood vessels is a key step during embryo development, but it also occurs in adults in physiologic and in pathologic conditions, such as retinopathy, rheumatoid arthritis, ischemia, and particularly tumor growth and metastasis (Carmeliet, Nat. Med. 9:653-660 (2003)). This pathological formation of new blood vessels is herein referred to as “invasive angiogenesis.” Basile et al., PNAS 103(24):9017-9022 (2006)). Angiogenesis is a frequently used strategy by which a wide variety of carcinomas may promote angiogenesis.

In accordance with the methods of the present invention, at least one AbCID, as defined elsewhere herein is used to promote a positive therapeutic response with respect to a malignant human cell. By “positive therapeutic response” with respect to cancer treatment is intended an improvement in the disease in association with the anti-tumor activity of these binding molecules. e.g. antibodies or fragments thereof, and/or an improvement in the symptoms associated with the disease. That is, an anti-proliferative effect, the prevention of further tumor outgrowths, a reduction in tumor size, a decrease in tumor vasculature, a reduction in the number of cancer cells, and/or a decrease in one or more symptoms associated with the disease can be observed. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the invention. Alternatively, an improvement in the disease may be categorized as being a partial response. By “partial response” is intended at least about a 50% decrease in all measurable tumor burden (i.e., the number of tumor cells present in the subject) in the absence of new lesions and persisting for at least one month. Such a response is applicable to measurable tumors only.

Tumor response can be assessed for changes in tumor morphology (i.e., overall tumor burden, tumor cell count, and the like) using screening techniques such as bioluminescent imaging, for example, luciferase imaging, bone scan imaging, and tumor biopsy sampling including bone marrow aspiration (BMA). In addition to these positive therapeutic responses, the subject undergoing therapy with the anti-CD100 binding molecule, e.g., an antibody or antigen-binding fragment thereof, may experience the beneficial effect of an improvement in the symptoms associated with the disease. For example, the subject may experience a decrease in the so-called B symptoms, e.g. night sweats, fever, weight loss, and/or urticaria.

The AbCIDs described herein may also find use in the treatment of inflammatory diseases and deficiencies or disorders of the immune system. Inflammatory diseases are characterized by inflammation and tissue destruction, or a combination thereof. By “anti-inflammatory activity” is intended a reduction or prevention of inflammation. “Inflammatory disease” includes any inflammatory immune-mediated process where the initiating event or target of the immune response involves non-self antigen(s), including, for example, alloantigens, xenoantigens, viral antigens, bacterial antigens, unknown antigens, or allergens. In one embodiment, the inflammatory disease is an inflammatory disorder of the peripheral or central nervous system. In another embodiment, the inflammatory disease is an inflammatory disorder of the joints.

Further, for purposes of the present invention, the term “inflammatory disease(s)” includes “autoimmune disease(s).” As used herein, the term “autoimmunity” is generally understood to encompass inflammatory immune-mediated processes involving “self” antigens. In autoimmune diseases, self antigen(s) trigger host immune responses. An autoimmune disease can result from an inappropriate immune response directed against a self antigen (an autoantigen), which is a deviation from the normal state of self-tolerance. In general, antibodies (particularly, but not exclusively, IgG antibodies), acting as cytotoxic molecules or as immune complexes, are the principal mediators of various autoimmune diseases, many of which can be debilitating or life-threatening.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the AbCID may experience the beneficial effect of an improvement in the symptoms associated with the disease.

The AbCID can be used in combination with at least one other cancer therapy, including, but not limited to, surgery or surgical procedures (e.g., splenectomy, hepatectomy, lymphadenectomy, leukophoresis, bone marrow transplantation, and the like); radiation therapy; chemotherapy, optionally in combination with autologous bone marrow transplant, or other cancer therapy; where the additional cancer therapy is administered prior to, during, or subsequent to the AbCID molecule, e.g., antibody or antigen binding fragment thereof, therapy. Thus, where the combined therapies comprise administration of an AbCID of the invention in combination with administration of another therapeutic agent, as with chemotherapy, radiation therapy, other anti-cancer antibody therapy, small molecule-based cancer therapy, or vaccine/immunotherapy-based cancer therapy, the methods of the invention encompass co-administration, using separate formulations or a single pharmaceutical formulation, or and consecutive administration in either order.

The AbCID molecules of the invention can be used in combination with any known therapies for cancer, autoimmune and inflammatory diseases, including any agent or combination of agents that are known to be useful, or which have been used or are currently in use, for treatment of autoimmune and inflammatory diseases. Thus, where the combined therapies comprise administration of an AbCID molecule in combination with administration of another therapeutic agent, the methods of the invention encompass co-administration, using separate formulations or a single pharmaceutical formulation, and consecutive administration in either order. In some embodiments of the invention, the AbCIDs described herein are administered in combination with immunosuppressive drugs or anti-inflammatory drugs, wherein the antibody and the therapeutic agent(s) may be administered sequentially, in either order, or simultaneously (i.e., concurrently or within the same time frame).

### Transcriptional Regulation

In some embodiments, provided herein is a method of treating a disease in an individual, comprising expressing in target cells in the individual the first and second CID components of a system according to any of the embodiments described herein wherein the CID is capable of regulating transcription of a target gene, wherein the expression level of the target gene in the target cells is associated with the disease, and administering to the individual the IMiD in a regimen effective to treat the disease.

In some embodiments, provided herein is a method of treating a disease in an individual, wherein the expression level of a target gene in the target cells is associated with the disease, comprising: (A) expressing in target cells in the individual (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises a DNA binding domain and the second adapter moiety comprises a transcriptional regulatory domain; or (2) the second adapter moiety comprises a DNA binding domain and the first adapter moiety comprises a transcriptional regulatory domain, wherein the first CID component and the second CID component are configured such that when dimerized in the presence of the IMiD to form the CID, the CID is capable of regulating transcription of the target gene; and (B) administering to the individual the IMiD in a regimen effective to treat the disease. In some embodiments, the first CID component further comprises a nuclear localization signal and the second CID component further comprises a nuclear localization signal. In some embodiments, (i) the transcriptional regulatory domain is a transcriptional activation domain, and the CID is capable of upregulating transcription of the target gene; or (ii) the transcriptional regulatory domain is a transcriptional repressor domain, and the CID is capable of downregulating transcription of the target gene. In some embodiments, the transcriptional regulatory domain is a VPR transcriptional activation domain. In some embodiments, the DNA binding domain is derived from a naturally occurring transcriptional regulator. In some embodiments, the DNA binding domain is derived from an RNA-guided endonuclease or a DNA-guided endonuclease. In some embodiments, the RNA-guided endonuclease or DNA-guided endonuclease is catalytically dead. In some embodiments, the DNA binding domain is derived from a catalytically dead Cas9 (dCas9). In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

### Cell Survival

In some embodiments, provided herein is a method of treating a disease in an individual, comprising: (A) administering to the individual an adoptive cell therapy for the disease comprising modified cells, wherein the modified cells express the first and second CID components of a system according to any of the embodiments described herein wherein the CID is capable of inducing target cell death; and (B) administering to the individual the IMiD in a regimen effective to (I) kill a predetermined amount of the adoptively transferred cells; or (II) maintain a predetermined amount of the adoptively transferred cells.

In some embodiments, provided herein is a method of treating a disease in an individual, comprising: (A) administering to the individual an adoptive cell therapy for the disease comprising modified cells, wherein the modified cells express (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the first adapter moiety and the second adapter moiety are together capable of inducing apoptosis in the target cell; and (B) administering to the individual the IMiD in a regimen effective to (I) kill a predetermined amount of the adoptively transferred cells; or (II) maintain a predetermined amount of the adoptively transferred cells. In some embodiments, the first adapter moiety and/or the second adapter moiety are derived from a caspase protein. In some embodiments, the adoptive cell therapy is a CAR T cell therapy. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

### Immune Modulation

In some embodiments, provided herein is a method of treating a disease characterized by a target cell in an individual, comprising: (A) administering to the individual modified T cells expressing the first and second CID components of a system according to any of the embodiments described herein wherein the CID is a heterodimeric CAR specific for a target antigen, and wherein the target antigen is expressed on the surface of the target cell; and (B) administering to the individual the IMiD in a regimen effective to treat the disease.

In some embodiments, provided herein is a method of treating a disease characterized by a target cell in an individual, comprising: (A) administering to the individual modified T cells expressing (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises (i) a transmembrane domain; (ii) a cytoplasmic co-stimulatory domain; and (iii) a cytoplasmic signaling domain; and the second adapter moiety comprises an extracellular antigen-binding moiety; or (2) the second adapter moiety comprises (i) a transmembrane domain; (ii) a cytoplasmic co-stimulatory domain; and (iii) a cytoplasmic signaling domain; and the first adapter moiety comprises an extracellular antigen-binding moiety; wherein the extracellular antigen-binding moiety specifically binds to the target antigen; and (B) administering to the individual the IMiD in a regimen effective to treat the disease. In some embodiments, the CID component comprising the extracellular antigen-binding moiety further comprises a secretory signal peptide. In some embodiments, the regimen is effective to treat the disease with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of treating a disease characterized by a target cell in an individual, comprising: (A) administering to the individual modified T cells expressing a first chemically induced dimer (CID) component comprising (i) a first binding moiety comprising cereblon or an IMiD-binding fragment or derivative thereof; and (ii) a first adapter moiety linked to the first binding moiety, wherein the first adapter moiety comprises (i) a transmembrane domain; (ii) a cytoplasmic co-stimulatory domain; and (iii) a cytoplasmic signaling domain; (B) administering to the individual a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the second adapter moiety comprises an extracellular antigen-binding moiety, and wherein the extracellular antigen-binding moiety specifically binds to the target antigen; and (C) administering to the individual the IMiD in a regimen effective to treat the disease. In some embodiments, the regimen is effective to treat the disease with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of treating a disease characterized by a target cell in an individual, comprising: (A) administering to the individual a first chemically induced dimer (CID) component comprising (i) a first binding moiety comprising cereblon or an IMiD-binding fragment or derivative thereof; and (ii) a first adapter moiety linked to the first binding moiety, wherein the first adapter moiety comprises an extracellular antigen-binding moiety, and wherein the extracellular antigen-binding moiety specifically binds to the target antigen; (B) administering to the individual modified T cells expressing a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the second adapter moiety comprises (i) a transmembrane domain; (ii) a cytoplasmic co-stimulatory domain; and (iii) a cytoplasmic signaling domain; and (C) administering to the individual the IMiD in a regimen effective to treat the disease. In some embodiments, the regimen is effective to treat the disease with fewer adverse effects in the individual as compared to a corresponding method comprising administration of CAR T cells expressing a conventional CAR comprising the corresponding CAR domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of treating a disease characterized by a target cell in an individual, comprising: (A) administering to the individual the first and second CID components of a system according to any of the embodiments described herein wherein the CID is a heterodimeric bispecific T cell engager capable of redirecting a T cell to the target cell; and (B) administering to the individual the IMiD in a regimen effective to treat the disease.

In some embodiments, provided herein is a method of treating a disease characterized by a target cell in an individual, comprising: (A) administering to the individual (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein (1) the first adapter moiety comprises a T cell antigen-binding moiety and the second adapter moiety comprises a target cell antigen-binding moiety; or (2) the second adapter moiety comprises a T cell antigen-binding moiety and the first adapter moiety comprises a target cell antigen-binding moiety; and (B) administering to the individual the IMiD in a regimen effective to treat the disease. In some embodiments, the T cell antigen-binding moiety is an antibody moiety that specifically binds to CD3. In some embodiments, the target cell antigen-binding moiety is an antibody moiety that specifically binds to a cell surface antigen associated with a diseased cell. In some embodiments, the diseased cell is a cancer cell. In some embodiments, the target cell antigen-binding moiety is an antibody moiety that specifically binds to CD19. In some embodiments, the regimen is effective to treat the disease with fewer adverse effects in the individual as compared to a corresponding method comprising administration of a conventional bispecific T cell engager comprising the corresponding bispecific T cell engager domains of the CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

In some embodiments, provided herein is a method of treating a disease characterized by a target cell in an individual, comprising: (A) expressing in T cells in the individual capable of recognizing and killing the target cell the first and second CID components of a system according to any of the embodiments described herein wherein the CID is a heterodimeric signaling molecule capable of modulating activation of the T cell; and (B) administering to the individual the IMiD in a regimen effective to treat the disease.

In some embodiments, provided herein is a method of treating a disease characterized by a target cell in an individual, comprising: (A) expressing in T cells in the individual capable of recognizing and killing the target cell (a) a first chemically induced dimer (CID) component comprising (i) a first binding moiety comprising cereblon or an IMiD-binding fragment or derivative thereof; and (ii) a first adapter moiety linked to the first binding moiety; and (b) a second CID component comprising (i) a second binding moiety, wherein the second binding moiety is an antibody moiety capable of specifically binding to a complex between the first binding moiety and an IMiD (e.g., thalidomide, lenalidomide, or pomalidomide); and (ii) a second adapter moiety linked to the second binding moiety, wherein the first adapter moiety comprises (i) a transmembrane domain; and (ii) a cytoplasmic co-stimulatory domain; and the second adapter moiety comprises (i) a transmembrane domain; and (ii) a cytoplasmic co-stimulatory domain; and (B) administering to the individual the IMiD in a regimen effective to treat the disease. In some embodiments, the T cells are CAR T cells. In some embodiments, the regimen is effective to treat the disease with fewer adverse effects in the individual as compared to a corresponding method comprising expression of a monomeric signaling molecule comprising the corresponding signaling domains of the CID in the T cells. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity. In some embodiments, the first binding moiety comprises the amino acid sequence of SEQ ID NO: 25 or a variant thereof having at least 85% identity to the amino acid sequence of SEQ ID NO: 25.

### Protein Degradation

In some embodiments, provided herein is a method of treating a disease characterized by a target cell in an individual, comprising: (A) administering to the individual T cells expressing the second CID component of a system according to any of the embodiments described herein wherein dimerization of the first and second CID components in the presence of an IMiD allows for ubiquitination of i) a CAR or cytokine (e.g., IL-2, IL-12, or IL-15) associated with the second CID component or ii) a fusion protein comprising the CAR or cytokine and the second CID component, and the ubiquitination of the CAR or cytokine or fusion protein thereof targets it for degradation, wherein the T cells are specific for a target antigen expressed on the surface of the target cell and comprise a first CID component comprising cereblon or an IMiD-binding fragment or derivative thereof capable of facilitating ubiquitination; and (B) administering to the individual the IMiD in a regimen effective to treat the disease.

In some embodiments, provided herein is a method of treating a disease characterized by a target cell in an individual, comprising: (A) administering to the individual T cells expressing a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a CAR specific for a target antigen expressed on the surface of the target cell, wherein the T cells are CAR T cells comprising a first CID component comprising cereblon or an IMiD-binding fragment or derivative thereof capable of facilitating ubiquitination; and (B) administering to the individual the IMiD in a regimen effective to treat the disease. In some embodiments, dimerization of the CID results in ubiquitination of the second CID component comprising the CAR such that it is targeted for degradation. In some embodiments, the first CID component is endogenous cereblon in the T cells. In some embodiments, the regimen is effective to treat the disease with fewer adverse effects in the individual as compared to a corresponding method comprising administration of the T cells in the absence of the IMiD, or administration of CAR T cells comprising a conventional form of the CAR that is not part of a CID. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of treating a disease characterized by a target cell in an individual, comprising: (A) administering to the individual T cells expressing a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a cytokine (e.g., IL-2, IL-12, or IL-15), wherein the T cells are specific for a target antigen expressed on the surface of the target cell and comprise a first CID component comprising cereblon or an IMiD-binding fragment or derivative thereof capable of facilitating ubiquitination; and (B) administering to the individual the IMiD in a regimen effective to treat the disease. In some embodiments, the T cells are CAR T cells comprising a CAR specific for the target antigen. In some embodiments, dimerization of the CID results in ubiquitination of the second CID component comprising the cytokine such that it is targeted for degradation. In some embodiments, the first CID component is endogenous cereblon in the T cells. In some embodiments, the regimen is effective to treat the disease with fewer adverse effects in the individual as compared to a corresponding method comprising administration of the T cells in the absence of the IMiD. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of treating a disease characterized by a target cell in an individual, comprising: (A) administering to the individual CAR T cells comprising a CAR specific for a target antigen expressed on the surface of the target cell and expressing a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a CAR-binding moiety capable of binding to the CAR present in the CAR T cells, wherein the CAR T cells comprise a first CID component comprising cereblon or an IMiD-binding fragment or derivative thereof capable of facilitating ubiquitination; and (B) administering to the individual the IMiD in a regimen effective to treat the disease. In some embodiments, dimerization of the CID results in ubiquitination of a CAR bound to the CAR-binding moiety such that the CAR is targeted for degradation. In some embodiments, the first CID component is endogenous cereblon in the CAR T cells. In some embodiments, the regimen is effective to treat the disease with fewer adverse effects in the individual as compared to a corresponding method comprising administration of the CAR T cells in the absence of the IMiD. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of treating a disease characterized by a target cell in an individual, comprising: (A) administering to the individual T cells expressing a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a cytokine-binding moiety capable of binding to a cytokine (e.g., IL-2, IL-12, or IL-15) present in the T cells, wherein the T cells are specific for a target antigen expressed on the surface of the target cell and comprise a first CID component comprising cereblon or an IMiD-binding fragment or derivative thereof capable of facilitating ubiquitination; and (B) administering to the individual the IMiD in a regimen effective to treat the disease. In some embodiments, the T cells are CAR T cells comprising a CAR specific for the target antigen. In some embodiments, dimerization of the CID results in ubiquitination of a cytokine bound to the cytokine-binding domain such that it is targeted for degradation. In some embodiments, the first CID component is endogenous cereblon in the T cells. In some embodiments, the regimen is effective to treat the disease with fewer adverse effects in the individual as compared to a corresponding method comprising administration of the T cells in the absence of the IMiD. In some embodiments, the IMiD is thalidomide. In some embodiments, the IMiD is lenalidomide. In some embodiments, the IMiD is pomalidomide. In some embodiments, the cytokine is IL-2, IL-12, or IL-15. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

In some embodiments, provided herein is a method of treating a disease in an individual, comprising a) expressing in target cells in the individual the second CID component of a system according to any of the embodiments described herein wherein dimerization of the first and second CID components allows for ubiquitination of a POI associated with the second CID component and the ubiquitination of the POI targets it for degradation, wherein a level of the POI in the target cells above a certain threshold is associated with the disease, and b) administering to the individual the IMiD in a regimen effective to treat the disease.

In some embodiments, provided herein is a method of treating a disease in an individual, comprising a) expressing in target cells in the individual a second CID component comprising i) a second binding moiety, wherein the second binding moiety is an antibody moiety capable of specifically binding to a complex between cereblon or an IMiD-binding fragment or derivative thereof and an IMiD (e.g., thalidomide, lenalidomide, or pomalidomide); and ii) a second adapter moiety linked to the second binding moiety, wherein the second adapter moiety comprises a POI-binding moiety capable of binding the POI, wherein a level of the POI in the target cells above a certain threshold is associated with the disease; and b) administering to the individual the IMiD in a regimen effective to treat the disease. In some embodiments, the POI-binding moiety is an antibody moiety specific for the POI. In some embodiments, the POI-binding moiety is a naturally occurring cognate binding partner of the POI or a POI-binding fragment or derivative thereof. In some embodiments, the IMiD is thalidomide. In some embodiments, the IMiD is lenalidomide. In some embodiments, the IMiD is pomalidomide. In some embodiments, the antibody moiety of the second CID component comprises a heavy chain variable domain and a light chain variable domain comprising HC-CDRs and LC-CDRs from a Fab-phage clone as shown in Table 1, or variants thereof having at least 85% identity.

### Pharmaceutical Compositions and Administration Methods

Methods of preparing and administering the AbCIDs, of the invention to a subject in need thereof are well known to or are readily determined by those skilled in the art. The route of administration of the AbCID may be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as being within the scope of the invention, an example of a form for administration would be a solution for injection, in particular for intravenous or intraarterial injection or drip. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, an AbCID of the invention can be delivered directly to the site of the adverse cellular population thereby increasing the exposure of the diseased tissue to the therapeutic agent.

As discussed herein, an AbCID of the invention may be administered in a pharmaceutically effective amount for the in vivo treatment of various cell-mediated diseases such as certain types of cancers, autoimmune diseases, inflammatory diseases including central nervous system (CNS) and peripheral nervous system (PNS) inflammatory diseases, and invasive angiogenesis. In this regard, it will be appreciated that the disclosed binding molecules of the invention will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of an AbCID conjugated or unconjugated, shall be held to mean an amount sufficient to achieve effective binding to a target and to achieve a benefit, e.g., to ameliorate symptoms of a disease or disorder or to detect a substance or a cell.

The pharmaceutical compositions used in this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and fat.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject invention, pharmaceutically acceptable carriers include, but are not limited to, 0.01-0.1 M and preferably 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g. glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences (Mack Publishing Co.) 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an AbCID by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in U.S. patent application Ser. No. 09/259,337. Such articles of manufacture will preferably have labels or package inserts indicating that the associated compositions are useful for treating a subject suffering from, or predisposed to a disease or disorder.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

The amount of AbCID that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

In keeping with the scope of the present disclosure, AbCIDs of the invention may be administered to a human or other animal in accordance with the aforementioned methods of treatment in an amount sufficient to produce a therapeutic effect. The AbCID of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of AbCID of the invention may prove to be particularly effective.

By “therapeutically effective dose or amount” or “effective amount” is intended an amount of AbCID that when administered brings about a positive therapeutic response with respect to treatment of a patient with a disease to be treated.

Therapeutically effective doses of the compositions of the present invention, for treatment of cell-mediated diseases such as certain types of cancers, e.g., leukemia, lymphoma; autoimmune diseases, e.g., arthritis, multiple sclerosis, inflammatory diseases including central nervous system (CNS) and peripheral nervous system (PNS) inflammatory diseases; and invasive angiogenesis, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

The amount of at least one AbCID to be administered is readily determined by one of ordinary skill in the art without undue experimentation given the disclosure of the present invention. Factors influencing the mode of administration and the respective amount of at least one AbCID include, but are not limited to, the severity of the disease, the history of the disease, and the age, height, weight, health, and physical condition of the individual undergoing therapy. Similarly, the amount of AbCID to be administered will be dependent upon the mode of administration and whether the subject will undergo a single dose or multiple doses of this agent.

In a further exemplary embodiment, cells of a subject are transfected with nucleic acids encoding a first CID component, a second CID component, fusions or derivatives thereof, or a combination of two or more of these elements, such that the cells produce the polypeptide(s) encoded therein. In various embodiments, a CID dimer is formed in the subject by administering to the subject an effective SMDA.

### Immunoassays

AbCIDs of the invention may used in immunoassays, e.g., they may be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl. 0.01 M sodium phosphate at pH 7.2.1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, (1994) Current Protocols in Molecular Biology (John Wiley & Sons, Inc., NY) Vol. 1 at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or .sup.125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, (1994) Current Protocols in Molecular Biology (John Wiley & Sons, Inc., NY) Vol. 1 at 10.8.1.

ELISAs comprise preparing antigen, coating the well of a 96-well microtiter plate with the antigen, adding the antibody of interest conjugated to a detectable compound such as an enzymatic substrate (e.g. horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, (1994) Current Protocols in Molecular Biology (John Wiley & Sons, Inc., NY) Vol. 1 at 11.2.1.

The binding affinity of a second binding moiety and its cognate binding partner, e.g., an antibody to an antigen, and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

AbCIDs, additionally, can be employed histologically, as in immunofluorescence, immunoelectron microscopy or non-immunological assays, for in situ detection of a selected protein. In situ detection may be accomplished by removing a histological specimen from a patient, and applying thereto a labeled AbCID, preferably applied by overlaying the labeled AbCID onto a biological sample. Through the use of such a procedure, it is possible to determine not only the presence of the selected protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays using an AbCID will typically comprise incubating a sample, such as a biological fluid, a tissue extract, freshly harvested cells, or lysates of cells which have been incubated in cell culture, in the presence of a detectably labeled AbCID, and detecting the bound antibody by any of a number of techniques well known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled AbCID. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of AbCID may be determined according to well-known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIACORE® offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE® code No. BR-1001-86; BIAtechnology Handbook, version AB (reprinted 1998), BIACORE® code No. BR-1001-84. SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIACORE®. measurements monitor interactions directly as they happen. The technique is well suited to determination of kinetic parameters. Comparative affinity ranking is simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (ka and kd, the rates of complex formation and dissociation, kd/ka=KD). The equilibrium phase provides information on the affinity of the analyte-ligand interaction (KD).

BIAevaluation software provides comprehensive facilities for curve fitting using both numerical integration and global fitting algorithms. With suitable analysis of the data, separate rate and affinity constants for interaction can be obtained from simple BIACORE® investigations. The range of affinities measurable by this technique is very broad ranging from mM to pM.

Epitope specificity is an important characteristic of a monoclonal antibody. Epitope mapping with BIACORE®, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two Abs to bind simultaneously to the same antigen. Abs directed against separate epitopes will bind independently, whereas MAbs directed against identical or closely related epitopes will interfere with each other's binding. These binding experiments with BIACORE® are straightforward to carry out.

In various embodiments BioLayer Interferometry (BLI) is utilized to assess binding, e.g., octet BLI.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different Abs to immobilized antigen. Peptides which interfere with binding of a given Ab are assumed to be structurally related to the epitope defined by that Ab.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise. Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor. N.Y., (1986); and in Ausubel et al. (1989) Current Protocols in Molecular Biology (John Wiley and Sons. Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

### Nucleic Acid Delivery

In some embodiments, any nucleic acid molecules used in the methods provided herein, e.g. a nucleic acid encoding an AbCID, are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Exemplary delivery methods and reagents are described in WO2018002719.

The present disclosure has been described above with reference to specific alternatives. However, other alternatives than the above described are equally possible within the scope of the disclosure. Different method steps than those described above, may be provided within the scope of the disclosure. The different features and steps described herein may be combined in other combinations than those described.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

In addition, where features or aspects of the disclosure are described in terms of Markush groups, those skilled in the art will recognize that the disclosure is also thereby described in terms of any individual member or subgroup of members of the Markush group.

Any of the features of an alternative of the first through eleventh aspects is applicable to all aspects and alternatives identified herein. Moreover, any of the features of an alternative of the first through eleventh aspects is independently combinable, partly or wholly with other alternatives described herein in any way, e.g., one, two, or three or more alternatives may be combinable in whole or in part. Further, any of the features of an alternative of the first through eleventh aspects may be made optional to other aspects or alternatives. Although described above in terms of various example alternatives and implementations, it should be understood that the various features, aspects and functionality described in one or more of the individual alternatives are not limited in their applicability to the particular alternative with which they are described, but instead may be applied, alone or in various combinations, to one or more of the other alternatives of the present application, whether or not such alternatives are described and whether or not such features are presented as being a part of a described alternative. Thus, the breadth and scope of the present application should not be limited by any of the above-described example alternatives.

All references cited herein are incorporated herein by reference in their entirety. To the extent publications and patents or patent applications incorporated by reference contradict the disclosure contained in the specification, the specification is intended to supersede and/or take precedence over any such contradictory material. To the extent publications and patents or patent applications incorporated by reference herein contradict the disclosure contained in the specification, the specification is intended to supersede and/or take precedence over any such contradictory material.

The details of one or more embodiments of the disclosure are set forth in the accompanying description below. Any materials and methods similar or equivalent to those described herein can be used in the practice or testing of the present disclosure. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### EXAMPLES

### Materials and Methods

### Small Molecule and Peptide Reagents

Thalidomide (Selleckchem), lenalidomide (MedChemExpress), and pomalidomide (Selleckchem) were used without further purification. For use, thalidomide, lenalidomide, and pomalidomide were each dissolved in DMSO as 10 mM stocks. Stocks were stored at −80° C. until used.

### Expression and Biotinylation of Cereblon

A vector containing a CDS encoding the IMiD-binding domain of cereblon (SEQ ID NO: 25) fused to a Tabaco Etch Virus (TEV) cut site, an AviTag, and a HIS6 tag was purchased from IDT (Integrated DNA technologies, USA). The CDS was cloned into the pMCSG7 vector (Kong, et al., Biomol. Ther., 21:423434 (2013)) using Gibson cloning. The sequence of the final construct was confirmed by sequencing of the entire gene. The plasmid was transformed into BL21(DE3) E. coli cells and a single colony was used to inoculate 1.5 L of 2×YT media containing carbenicillin (100 μg/mL). The culture was grown at 37° C. to an OD600 of 1-1.2, cooled to 18° C. for 1 h and then induced at 18° C. overnight with 0.5 mM IPTG. Cells were harvested by centrifugation and the pellet were stored at −80° C.

For protein purification, the pellet was thawed at 0° C. and then re-suspended in 10 mL of lysis buffer (50 mM Tris, pH 8.0, 200 mM NaCl, 20 mM imidazole) supplemented with PMSF (100 μg/mL). The cells were lysed using a micro-fluidizer and the lysate was cleared by centrifugation at 4° C. The cleared lysate was added to 400 μL of Ni-NTA Superflow resin (Qiagen) and rotated at 4° C. for 1 h. The resin was washed (3×) with lysis buffer and then transferred to a spin column. The purified protein was eluted with elution buffer (50 mM Tris, pH 8.0, 200 mM NaCl, 600 mM imidazole). Fractions were analyzed by SDS-PAGE and those that were found to be >95% pure were pooled, exchanged into storage buffer (25 mM Tris, pH 8.0, 150 mM NaCl, 1 mM DTT) and concentrated.

The purified cereblon protein was biotinylated on its AviTag using the standard protocol provided by Avidity. Biotinylation was monitored by intact protein mass spectrometry on a Xevo G2-XS Mass Spectrometer (Waters) and found to be quantitative. The biotinylated cereblon was then purified on Ni-NTA as described above, separated into aliquots, snap-frozen and stored at −80° C. for later use.

### Phage Display Selections and Phage Tittering

All phage selections were done according to previously established protocols (Seiler, et al., Nucleic Acids Res., 42:D12531260 (2014). Briefly, selections with antibody phage library F were performed using biotinylated cereblon captured with streptavidin-coated magnetic beads (Promega). Prior to each selection, the phage pool was incubated with 1 μM of cereblon immobilized on streptavidin beads in the absence of thalidomide, lenalidomide, or pomalidomide in order to deplete the library of any binders to the unbound form of cereblon. Subsequently, the beads were removed and in separate selections thalidomide, lenalidomide, or pomalidomide was added to the phage pool at a concentration of 5 μM. In total, four rounds of selection were performed with decreasing amounts of cereblon antigen (100 nM, 50 nM, 10 nM and 10 nM). To reduce the deleterious effects of nonspecific binding phage, we employed a “catch and release” strategy, where specific cereblon-binding Fab-phage were selectively eluted from the magnetic beads by the addition of 2 μg/mL TEV protease. Individual phage clones from the fourth round of selection were analyzed for sequencing.

Phage titers were performed according to standard protocols. Briefly, TEV eluted phage were used to infect log-phase XL1-Blue E. coli cells (Stratagene). Infected cells were incubated at room temperature for 20 minutes on an orbital shaker. Cells were then serially diluted and spotted on LB agar-plates with carbenicillin (50 μg/mL) and incubated overnight at 37° C. Phage titers were measured for each round of selections against both cereblon/IMiD complex and against unbound cereblon.

### Expression of Fabs

Fabs were expressed according to a previously described protocol (Seiler, et al., Nucleic Acids Res., 42:D1253-1260 (2014)). Briefly, C43 (DE3) Pro+ E. coli containing expression plasmids were grown in 2×YT at 37° C. to an OD600 of 0.6-0.8 and then Fab expression was induced by the addition of 1 mM IPTG. Incubation temperature was subsequently reduced to 30° C. and the cultures were allowed to shake for 16-18 h. Cells were harvested by centrifugation and Fabs were purified by Protein A affinity chromatography. Fab purity and integrity was assessed by SDS-PAGE and intact protein mass spectrometry using a Xevo G2-XS Mass Spectrometer (Waters).

### Fab ELISAs

ELISAs were performed according to standard protocols. Briefly, 96-well Maxisorp plates were coated with NeutrAvidin (10 μg/ml) overnight at 4° C. and subsequently blocked with BSA (2% w/v) for 1 h at 20′C. 20 nM of biotinylated cereblon was captured on the NeutrAvidin-coated wells for 30 minutes followed by the addition of various concentrations of Fab with either 5 μM thalidomide, lenalidomide, or pomalidomide or 0.05% DMSO for 30 minutes. The bound Fabs were then detected using a horseradish peroxidase (HRP)-conjugated anti-Fab monoclonal antibody (Jackson ImmunoResearch 109-036-097).

### Binding Kinetics Analysis

Biolayer interferometery data were measured using an Octet RED384 instrument (ForteBio). Biotinylated cereblon was immobilized on a Streptavidin (SA) biosensor using a 200 nM solution. Serial dilutions of Fabs in kinetics buffer (PBS, pH 7.4, 0.05% Tween-20, 0.2% BSA, 10 μM biotin) with IMiD (1 μM) or vehicle (0.05% DMSO) were used as analyte. Affinity (KD) and kinetic parameters (kon and koff) were calculated from a global fit (1:1) of the data using the Octet RED384 software.

### Vector Generation for Cellular Assays

Fab TC1 was converted into a previously described single-chain Fab construct using Gibson cloning (Hornsby, et al., Mol. Cell. Proteomics, 14:2833-2847 (2015)) and subsequently fused to eGFP by Gibson cloning into a pcDNA3.1 vector to produce an expression vector encoding an scTC1-eGFP fusion protein.

### Culturing of Cell Lines

K562 and HEK293T cells utilized were from frozen stocks maintained by the Wells lab. The cell lines were not authenticated before use. No test for mycoplasma contamination was performed. Cells expressing scTC1-eGFP were transfected with FugeneHD transfection reagent purchased from Promega using the manufacturer's protocol. Unless otherwise noted all Jurkat and K562 cells lines were cultured in RPMI supplemented with 10% FBS and 1× Pen/Strep. CD19⁺ K562 cells were maintained in puromycin (2 μg/mL). HEK293T cells containing the Gal4-UAS-Fluc operon were maintained in High Glucose DMEM supplemented with 10% FBS, 1× Pen/Strep, and puromycin (2 μg/mL). All cell lines were cultured at 37° C. under 5% CO2.

### Example 1: Selection of Chemical-Epitope-Selective Antibodies

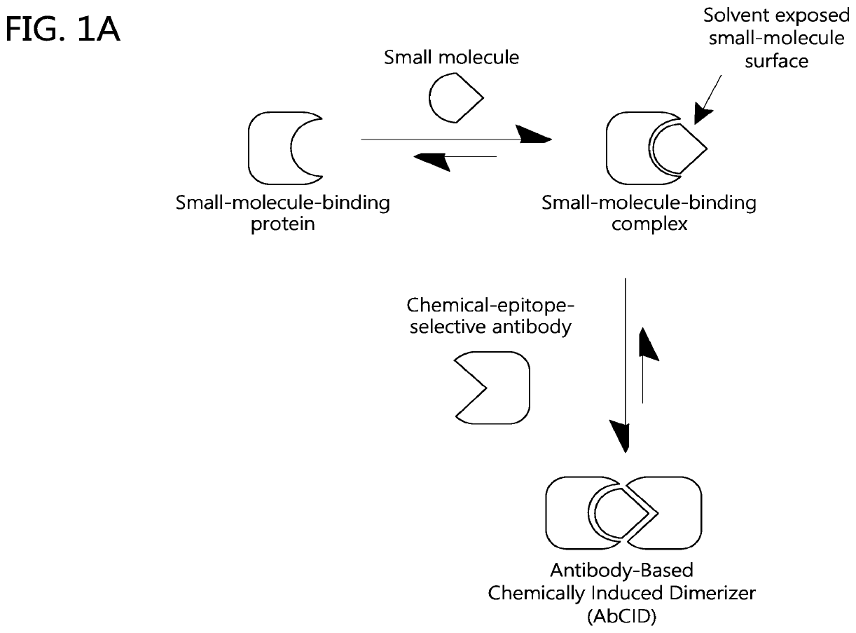

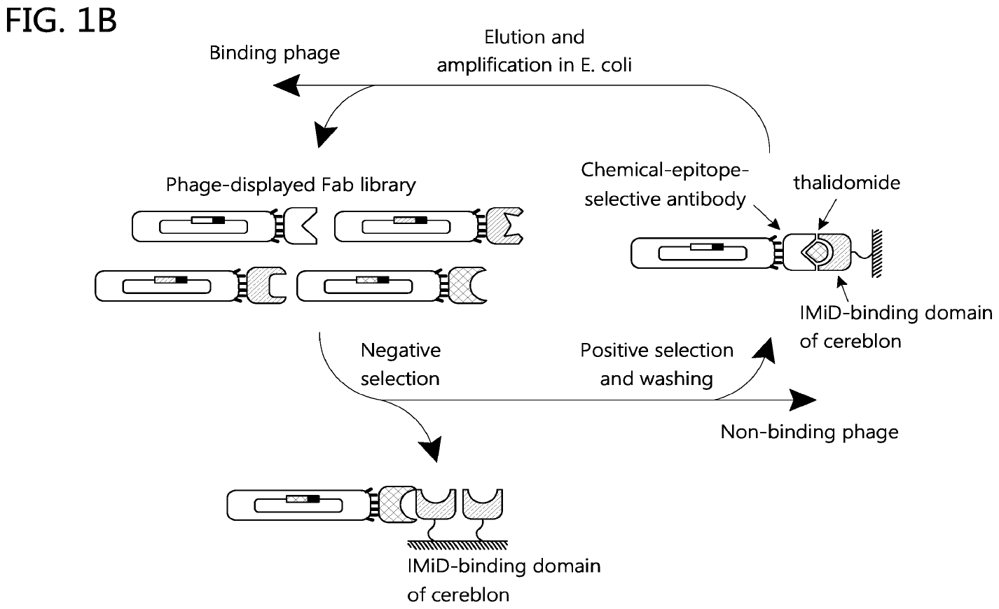

To identify unique chemical-epitope-selective antibodies, we utilized the IMiD-binding domain of cereblon (SEQ ID NO: 25) fused to a Tabaco Etch Virus (TEV) cut site, an AviTag, and a HIS6 tag. Biotinylated cereblon IMiD-binding domain was immobilized on streptavidin resin and used for phage selections with a previously developed synthetic antibody-fragment library and selection strategy as depicted in FIG. 1B for each of thalidomide-bound cereblon IMiD-binding domain, lenalidomide-bound cereblon IMiD-binding domain, and pomalidomide-bound cereblon IMiD-binding domain (Hornsby, et al., Mol. Cell. Proteomics, 14:2833-2847 (2015)). During each round of selection, the phage library was first subjected to stringent counter selection against the cereblon IMiD-binding domain in the absence of IMiD, thereby removing any Fab-phage that was not selective for the IMiD-bound form. Positive selections were then performed in the presence of saturating amounts of the respective IMiD (1 μM thalidomide, lenalidomide, or pomalidomide), ensuring that the majority of the cereblon IMiD-binding domain was bound to an IMiD. A total of four rounds of selection were performed. After round four, 48 Fab-phage clones were identified from each of the selections, and these were further characterized by competition ELISA in the presence or absence of the respective IMiD to identify Fabs that selectively bound to the IMiD-bound form of the cereblon IMiD-binding domain. The competition ELISA conditions included a) immobilized IMiD-bound cereblon IMiD-binding domain, b) immobilized IMiD-bound cereblon IMiD-binding domain+20 nM soluble competitor, c) immobilized unbound cereblon IMiD-binding domain, and d) immobilized BSA control. Fab binding was measured as OD₄₅₀. For each clone, a competition selectivity ratio was calculated as (competitor Fab binding)/(IMiD-cereblon Fab binding) and an ELISA intensity was calculated as (IMiD-cereblon IMiD-binding domain Fab binding)−(cereblon IMiD-binding domain Fab binding). Results of the competition ELISA assays for thalidomide-cereblon Fabs, lenalidomide-cereblon Fabs, and pomalidomide-cereblon Fabs are shown in FIGS. 2A-2F, respectively. The hits (light grey), determined as Fabs with a competition selectivity ratio less than 0.5 and ELISA intensity greater than 0.5, were sequenced to identify clones with unique sequences. A total of five Fab-phage clones with unique sequences in the complementarity-determining regions (CDRs) of the Fab were identified, two for the screen with thalidomide-cereblon IMiD-binding domain, one for the screen with lenalidomide-cereblon IMiD-binding domain, and two for the screen with pomalidomide-cereblon IMiD-binding domain (Table 1).

### Example 2: Specificity of Identified Antibodies for the IMiD-Bound Forms of Cereblon

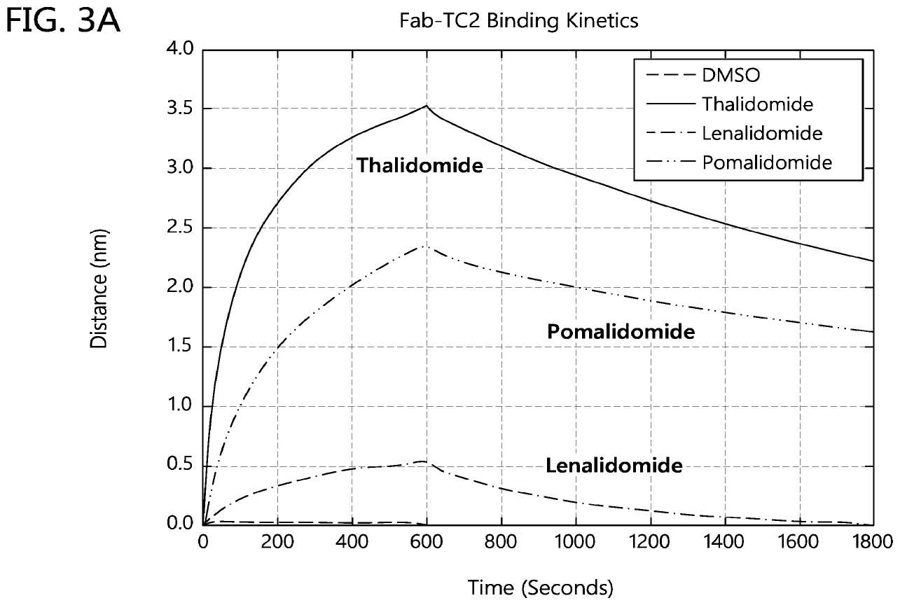

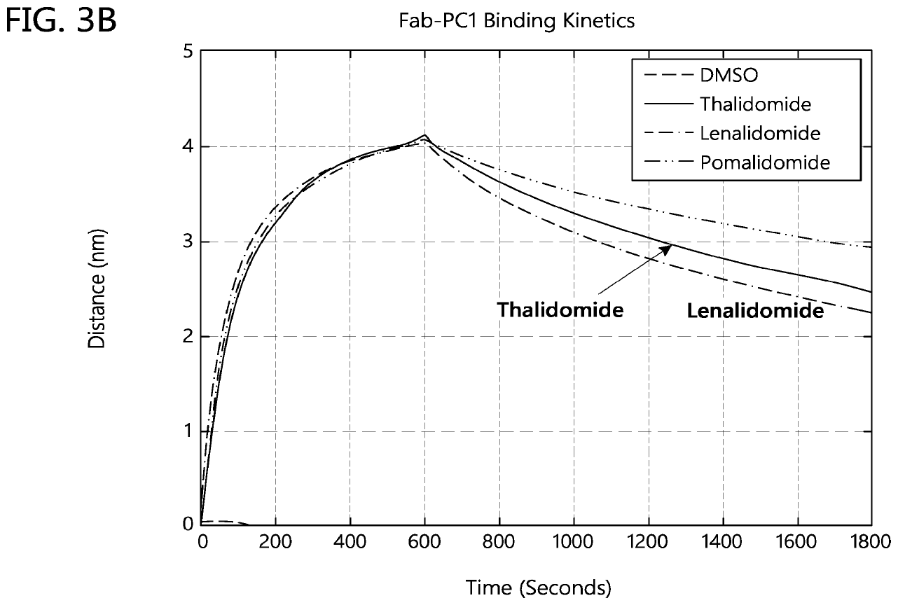

The unique Fabs from Example 1 were sub-cloned into a bacterial expression vector, expressed, and purified (Hornsby, et al., Mol. Cell. Proteomics, 14:2833-2847 (2015)). Enzyme-linked immunosorbent assays (ELISA) with the cereblon IMiD-binding domain (SEQ ID NO: 25) in the presence or absence of thalidomide, lenalidomide, or pomalidomide showed that all except one of the Fabs (Fab-phage clone TC2) was able to bind the cereblon IMiD-binding domain bound to a different IMiD than the one used for selection (FIG. 2G). To further profile the Fabs, we characterized the kinetics of cereblon IMiD-binding domain binding for Fab-TC2 and Fab-PC1 in the presence or absence of their respective IMiD by biolayer interferometry (FIGS. 3A and 3B) (Shah, et al., J. Vis. Exp., e51383 (2014)). Both Fab-TC2 and Fab-PC1 showed potent and reversible binding to the cereblon IMiD-binding domain in the presence of thalidomide, lenalidomide, and pomalidomide, but no significant binding to the cereblon IMiD-binding domain alone, though Fab-TC2 had less potent binding to the cereblon IMiD-binding domain in the presence of lenalidomide or pomalidomide as compared to the cereblon IMiD-binding domain in the presence of thalidomide, the IMiD used for its selection.

### Example 3: Fab-TC1 AbCID for IMiD-Induced Degradation of Fusion Proteins

This Example demonstrates the function of an AbCID format useful for SMDA-induced degradation of fusion proteins containing a Fab that binds cereblon in the presence of an IMiD (the SMDA). Cereblon is part of the E3 ubiquitin ligase complex, which is capable of ubiquinating proteins and targeting them for proteasomal degradation. Addition of the IMiD to a cell expressing the fusion protein can allow for ubiquitin ligase complexes in the cell containing cereblon to bind to the fusion protein, leading to its ubiquitination and degradation.

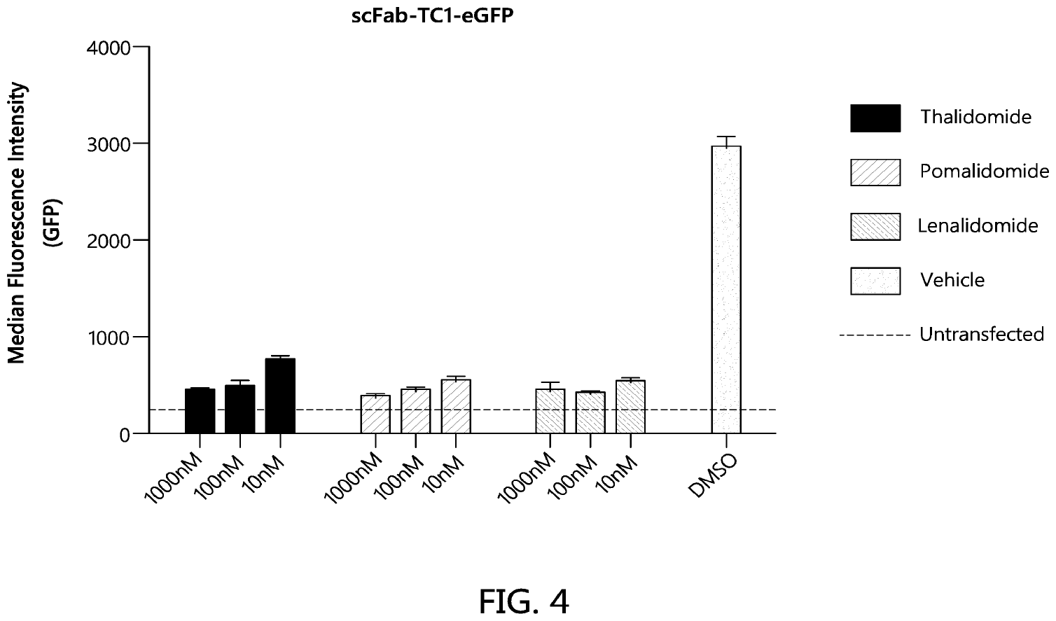

Fab-TC1 was adapted to a single-chain Fab (scFab) format in which the light and heavy chains are genetically fused as a single polypeptide (Koerber, et al., J. Mol. Biol., 427:576-586 (2015)), and this scFab was fused to eGFP to generate an scFab-TC1-eGFP fusion protein. HEK 293T cells (ATCC® CRL-3216™) were transfected to express scFab-TC1-eGFP. Transfected cells were treated with thalidomide, lenalidomide, or pomalidomide at 1000 nM, 100 nM, or 10 nM for 20 hours, after which GFP fluorescence intensity was measured by flow cytometry. Treatment with DMSO was included as a negative control. As shown in FIG. 4, treatment with thalidomide, lenalidomide, or pomalidomide resulted in reduced GFP expression compared to the DMSO control, indicating that degradation of the scFab-TC1-eGFP fusion protein in these cells was inducible by addition of the AbCID SMDA. These results support that the AbCIDs described herein can be used for tunable control of biological systems in living cells.

SEQUENCE LISTING

| SEQ | | |
|---|---|---|
| ID | | |
| NO | Sequence | Description |
| 1 | ISSYSI | Fab-TC1 HC-CDR1 (cereblon + |
| | | thalidomide) |
| 2 | VSSSSI | Fab-TC2 HC-CDR1 (cereblon + |
| | | thalidomide) |
| 3 | FSSSSI | Fab-LC1 HC-CDR1 (cereblon + |
| | | lenalidomide) |
| 4 | VSSYSI | Fab-PC1 HC-CDR1 (cereblon + |
| | | pomalidomide) |
| 5 | VSYSSI | Fab-PC2 HC-CDR1 (cereblon + |
| | | pomalidomide) |
| 6 | SISPSYGYTS | Fab-TC1 HC-CDR2 (cereblon + |
| | | thalidomide) |
| 7 | SIYSYYGSTY | Fab-TC2 HC-CDR2 (cereblon + |
| | | thalidomide) |
| 8 | YISSSSGSTS | Fab-LC1 HC-CDR2 (cereblon + |
| | | lenalidomide) |
| 9 | SISSYSGSTS | Fab-PC1 HC-CDR2 (cereblon + |
| | | pomalidomide) |
| 10 | SIYSYSGSTS | Fab-PC2 HC-CDR2 (cereblon + |
| | | pomalidomide) |
| 11 | SYYWQYYYQFGYPFGF | Fab-TC1 HC-CDR3 (cereblon + |
| | | thalidomide) |
| 12 | TDSWYYYRYGGM | Fab-TC2 HC-CDR3 (cereblon + |
| | | thalidomide) |
| 13 | SEYPYSYWYMYGYPVGGF | Fab-LC1 HC-CDR3 (cereblon + |
| | | lenalidomide) |
| 14 | SYFVEYYYYYGWPWGL | Fab-PC1 HC-CDR3 (cereblon + |
| | | pomalidomide) |
| 15 | SSYSYWYYIMYGYWFAM | Fab-PC2 HC-CDR3 (cereblon + |
| | | pomalidomide) |
| 16 | GSQSQMPF | Fab-TC1 LC-CDR3 (cereblon + |
| | | thalidomide) |
| 17 | YSWWWMPI | Fab-TC2 LC-CDR3 (cereblon + |
| | | thalidomide) |
| 18 | WGYYLI | Fab-LC1 LC-CDR3 (cereblon + |
| | | lenalidomide) |
| 19 | SSYSYLF | Fab-PC1 LC-CDR3 (cereblon + |
| | | pomalidomide) |
| 20 | SDSMPV | Fab-PC2 LC-CDR3 (cereblon + |
| | | pomalidomide) |
| 21 | RASQSVSSAVA | LC-CDR1 |
| 22 | SASSLYS | LC-CDR2 |
| 23 | EISEVQLVESGGGLVQPGGSLRLSCAASGFNF | Heavy Chain Scaffold |
| | SSSSIHWVRQAPGKGLEWVASISSSYGYTYY | |
| | ADSVKGRFTISADTSKNTAYLQMNSLRAEDT | |
| | AVYYCARTVRGSKKPYFSGWAMDYWGQGT | |
| | LVTVSSASTKGPSVFPLAPSSKSTSGGTAALG | |
| | CLVKDYFPEPVTVSWNSGALTSGVHTFPAVL | |
| | QSSGLYSLSSVVTVPSSSLGTQTYICNVNHKP | |
| | SNTKVDKKVEPKSCDKTHTGGSHHHHHH | |
| 24 | SDIQMTQSPSSLSASVGDRVTITCRASQSVSS | Light Chain Scaffold |
| | AVAWYQQKPGKAPKLLIYSASSLYSGVPSRF | |
| | SGSRSGTDFTLTISSLQPEDFATYYCQQSSYS | |
| | LITFGQGTKVEIKRTVAAPSVFIFPPSDSQLKS | |
| | GTASVVCLLNNFYPREAKVQWKVDNALQSG | |
| | NSQESVTEQDSKDSTYSLSSTLTLSKADYEK | |
| | HKVYACEVTHQGLSSPVTKSFNRGECGGSD | |
| | YKDDDDK | |
| 25 | TSLCCKQCQETEITTKNEIFSLSLCGPMAAYV | IMiD-binding domain of cereblon |
| | NPHGYVHETLTVYKACNLNLIGRPSTEHSWF | |
| | PGYAWTVAQCKICASHIGWKFTATKKDMSP | |
| | QKFWGLTRSALLPTIP | |

What is claimed is:

1. A system comprising:
(a) a first chemically induced dimer (CID) component comprising a first binding moiety capable of interacting with an immunomodulatory imide drug (IMiD) selected from thalidomide and analogs thereof to form a complex between the first CID component and the IMiD, or a first nucleic acid encoding polypeptide components of the first CID component; and
(b) a second CID component comprising a second binding moiety that specifically binds to the complex between the first CID component and the IMiD, or a second nucleic acid encoding polypeptide components of the second CID component.

2. The system of claim 1, wherein the first binding moiety comprises cereblon or an IMiD-binding fragment or derivative thereof.

3. The system of claim 1 or 2, further comprising the IMiD, wherein the second CID component is bound to a complex between the IMiD and the first CID component at a site of the complex comprising at least a portion of the IMiD and a portion of the first binding moiety.

4. The system of any one of claims 1-3, wherein the site of the complex comprising at least a portion of the IMiD and a portion of the first binding moiety is an interface between the IMiD and a binding site of the first binding moiety for the IMiD, comprising at least one atom of the IMiD and one atom of the first binding moiety.

5. The system of any one of claims 1-4, wherein the second binding moiety is an antibody moiety that specifically binds to a chemical-epitope comprising at least a portion of the IMiD and a portion of the first binding moiety.

6. The system of any one of claims 1-5, wherein the second binding moiety is an antibody moiety comprising heavy chain and light chain complementarity determining regions (CDRs) from a Fab-phage clone according to Table 1.

7. The system of any one of claims 1-6, wherein the IMiD is thalidomide, lenalidomide, or pomalidomide.

8. The system of any one of claims 1-7, wherein the first binding domain comprises the amino acid sequence of SEQ ID NO: 25 or a variant thereof having at least 85% identity to the amino acid sequence of SEQ ID NO: 25.

9. The system of any one of claims 1-8, wherein the first CID component further comprises a first adapter moiety linked to the first binding moiety and/or the second CID component further comprises a second adapter moiety linked to the second binding moiety.

10. The system of claim 9, wherein
(a) the first adapter moiety comprises a DNA binding domain and the second adapter moiety comprises a transcriptional regulatory domain; or
(b) the second adapter moiety comprises a DNA binding domain and the first adapter moiety comprises a transcriptional regulatory domain,
wherein the first CID component and the second CID component are configured such that when dimerized in the presence of the IMiD to form the CID, the CID is capable of regulating transcription of a target gene.

11. The system of claim 10, wherein
(a) the transcriptional regulatory domain is a transcriptional activation domain, and the CID is capable of upregulating transcription of the target gene; or
(b) the transcriptional regulatory domain is a transcriptional repressor domain, and the CID is capable of downregulating transcription of the target gene.

12. The system of claim 10 or 11, wherein the DNA binding domain is derived from a naturally occurring transcriptional regulator.

13. The system of claim 10 or 11, wherein the DNA binding domain is derived from an RNA-guided endonuclease or a DNA-guided endonuclease.

14. The system of claim 13, wherein the RNA-guided endonuclease or DNA-guided endonuclease is catalytically dead.

15. The system of claim 14, wherein the DNA binding domain is derived from a catalytically dead Cas9 (dCas9).

16. The system of claim 9, wherein the first CID component and the second CID component are configured such that when dimerized in the presence of the IMiD to form a CID associated with a target cell, the CID is capable of inducing target cell death.

17. The system of claim 16, wherein the first adapter moiety and the second adapter moiety are together capable of inducing apoptosis in the target cell.

18. The system of claim 17, wherein the first adapter moiety and/or the second adapter moiety are derived from a caspase protein.

19. The system of claim 18, wherein the first adapter moiety and the second adapter moiety are derived from caspase-9.

20. The system of any one of claims 16-19, wherein the target cell is an engineered cell adoptively transferred to an individual.

21. The system of claim 20, wherein the target cell is a T cell expressing a chimeric antigen receptor (CAR).

22. The system of claim 9, wherein the first CID component and the second CID component are configured such that when dimerized in the presence of the IMiD to form a CID associated with a T cell, the CID is a heterodimeric CAR capable of activating the T cell upon binding a target antigen.

23. The system of claim 22, wherein
(a) the first adapter moiety comprises (i) a transmembrane domain; (ii) a cytoplasmic co-stimulatory domain; and (iii) a cytoplasmic signaling domain; and the second adapter moiety comprises an extracellular antigen-binding moiety; or
(b) the second adapter moiety comprises (i) a transmembrane domain; (ii) a cytoplasmic co-stimulatory domain; and (iii) a cytoplasmic signaling domain; and the first adapter moiety comprises an extracellular antigen-binding moiety;
wherein the extracellular antigen-binding moiety specifically binds to the target antigen.

24. The system of claim 23, wherein the CID component comprising the extracellular antigen-binding moiety further comprises a secretory signal peptide.

25. The system of claim 22, wherein
(a) the first adapter moiety comprises (i) a cytoplasmic co-stimulatory domain or a cytoplasmic signaling domain; (ii) a transmembrane domain; and (iii) an extracellular antigen-binding moiety; and the second adapter moiety comprises a cytoplasmic co-stimulatory domain or a cytoplasmic signaling domain; or
(b) the second adapter moiety comprises (i) a cytoplasmic co-stimulatory domain or a cytoplasmic signaling domain; (ii) a transmembrane domain; and (iii) an extracellular antigen-binding moiety; and the first adapter moiety comprises a cytoplasmic co-stimulatory domain or a cytoplasmic signaling domain;
wherein the extracellular antigen-binding moiety specifically binds to the target antigen.

26. The system of claim 22, wherein the first adapter moiety comprises (i) a cytoplasmic co-stimulatory domain or a cytoplasmic signaling domain; and (ii) a transmembrane domain; and the second adapter moiety comprises (i) a cytoplasmic co-stimulatory domain or a cytoplasmic signaling domain; and (ii) a transmembrane domain; wherein the first or second CID component further comprises an extracellular antigen-binding moiety linked to its binding moiety; and wherein the extracellular antigen-binding moiety specifically binds to the target antigen.

27. The system of claim 25 or 26, wherein the first and second CID components together comprise a cytoplasmic co-stimulatory domain and a cytoplasmic signaling domain.

28. The system of claim 9, wherein the first CID component and the second CID component are configured such that when dimerized in the presence of the IMiD to form a CID, the CID is a heterodimeric bispecific T cell engager capable of redirecting a T cell to a target cell.

29. The system of claim 28, wherein
(a) the first adapter moiety comprises a T cell antigen-binding moiety and the second adapter moiety comprises a target cell antigen-binding moiety; or
(b) the second adapter moiety comprises a T cell antigen-binding moiety and the first adapter moiety comprises a target cell antigen-binding moiety.

30. The system of claim 29, wherein the T cell antigen-binding moiety is an antibody moiety that specifically binds to CD3.

31. The system of claim 29 or 30, wherein the target cell antigen-binding moiety is an antibody moiety that specifically binds to a cell surface antigen associated with a diseased cell.

32. The system of claim 31, wherein the diseased cell is a cancer cell.

33. The system of claim 31 or 32, wherein the target cell antigen-binding moiety is an antibody moiety that specifically binds to CD19.

34. The system of claim 9, wherein the first CID component and the second CID component are configured such that when dimerized in the presence of the IMiD to form a CID associated with an immune cell, the CID is a heterodimeric signaling molecule capable of modulating activation of the immune cell.

35. The system of claim 34, wherein the first adapter moiety comprises (i) a transmembrane domain; and (ii) a cytoplasmic co-stimulatory domain; and the second adapter moiety comprises (i) a transmembrane domain; and (ii) a cytoplasmic co-stimulatory domain.

36. The system of claim 35, wherein the first adapter moiety further comprises a cytoplasmic signaling domain and/or the second adapter moiety further comprises a cytoplasmic signaling domain.

37. The system of any one of claims 34-36, wherein the immune cell is a T cell.

38. The system of claim 37, wherein the T cell is a CART cell.

39. The system of any one of claims 1-8, wherein the first CID component and the second CID component are configured such that when dimerized in the presence of the IMiD to form a CID in a cell, association of the second CID component with the first CID component allows for ubiquitination of the second CID component or a protein-of-interest (POI) associated with the second CID component.

40. The system of claim 39, wherein dimerization of the CID results in ubiquitination of the second CID component or associated POI such that it is targeted for degradation.

41. The system of claim 39 or 40, wherein the first CID component is endogenous cereblon in the cell.

42. The system of any one of claims 39-41, wherein the second CID component is part of a fusion protein comprising the second CID component fused to a POI.

43. The system of claim 42, wherein the POI is a therapeutic protein, a CAR, or a cytokine.

44. The system of any one of claims 39-41, wherein the second CID component comprises a second adapter moiety comprising a POI-binding moiety capable of binding the POI.

45. The system of claim 44, wherein the POI is a pathogen-associated protein, an aberrantly expressed endogenous protein, a CAR, or a cytokine.

46. A method of modulating the expression of a target gene in a cell, comprising expressing the first and second CID components of the system of any one of claims 10-15 in the cell and modifying the amount of the IMiD in the cell to modulate the expression of the target gene.

47. A method of treating a disease in an individual, comprising:
(A) expressing the first and second CID components of the system of any one of claims 10-15 in target cells in an individual, wherein the expression level of the target gene in the target cells is associated with the disease; and
(B) administering to the individual the IMiD in a regimen effective to treat the disease.

48. Nucleic acid encoding the first and second CID components of the system of any one of claims 10-15.

49. A cell comprising the first and second CID components of the system of any one of claims 10-15.

50. A method of controlling the survival of target cells in an individual, comprising:
(A) expressing the first and second CID components of the system of any one of claims 16-21 in the target cells; and
(B) administering to the individual the IMiD in a regimen effective to (I) kill a predetermined amount of the target cells; or (II) maintain a predetermined amount of the target cells.

51. The method of claim 50, wherein the target cells are part of an adoptive cell therapy in the individual.

52. The method of claim 51, wherein the target cells are CAR T cells.

53. A method of treating a disease in an individual, comprising:
(A) administering to the individual an adoptive cell therapy for the disease comprising modified cells, wherein the modified cells express the first and second CID components of the system of any one of claims 16-21; and
(B) administering to the individual the IMiD in a regimen effective to (I) kill a predetermined amount of the adoptively transferred cells; or (II) maintain a predetermined amount of the adoptively transferred cells.

54. The method of claim 53, wherein the adoptive cell therapy is a CAR T cell therapy.

55. Nucleic acid encoding the first and second CID components of the system of any one of claims 16-21.

56. A cell comprising the first and second CID components of the system of any one of claims 16-21.

57. The cell of claim 56, wherein the cell is part of an adoptive cell therapy.

58. The cell of claim 57, wherein the cell is a CART cell.

59. A method of modulating an immune response to a target cell in an individual, comprising:
(A) administering to the individual modified T cells expressing the first and second CID components of the system of any one of claims 22-27, wherein the target antigen is expressed on the surface of the target cell; and
(B) administering to the individual the IMiD in a regimen effective to modulate an immune response to the target cell.

60. A method of modulating an immune response to a target cell in an individual, comprising:
(A) administering to the individual modified T cells expressing the CID component of the system of claim 23 comprising the cytoplasmic signaling domain;
(B) administering to the individual the CID component of the system of claim 23 comprising the extracellular antigen-binding moiety, wherein the target antigen is expressed on the surface of the target cell; and
(C) administering to the individual the IMiD in a regimen effective to modulate an immune response to the target cell.

61. The method of claim 59 or 60, wherein the regimen is effective to maintain an immune response to the target cell with fewer adverse effects in the individual as compared to a corresponding method comprising administration of CAR T cells expressing a conventional CAR comprising the corresponding CAR domains of the CID.

62. A method of treating a disease characterized by a target cell in an individual, comprising:
(A) administering to the individual modified T cells expressing the first and second CID components of the system of any one of claims 22-27, wherein the target antigen is expressed on the surface of the target cell; and
(B) administering to the individual the IMiD in a regimen effective to treat the disease.

63. A method of treating a disease characterized by a target cell in an individual, comprising:
(A) administering to the individual modified T cells expressing the CID component of the system of claim 23 comprising the cytoplasmic signaling domain;
(B) administering to the individual the CID component of the system of claim 23 comprising the extracellular antigen-binding moiety, wherein the target antigen is expressed on the surface of the target cell; and
(C) administering to the individual the IMiD in a regimen effective to treat the disease.

64. The method of claim 62 or 63, wherein the regimen is effective to treat the disease with fewer adverse effects in the individual as compared to a corresponding method comprising administration of CAR T cells expressing a conventional CAR comprising the corresponding CAR domains of the CID.

65. Nucleic acid encoding the first and second CID components of the system of any one of claims 22-27.

66. A T cell comprising the first and second CID components of the system of any one of claims 22-27.

67. A T cell comprising the CID component of the system of claim 23 comprising the cytoplasmic signaling domain.

68. A method of modulating an immune response to a target cell in an individual, comprising:
(A) administering to the individual the first and second CID components of the system of any one of claims 28-33; and
(B) administering to the individual the IMiD in a regimen effective to modulate an immune response to the target cell.

69. The method of claim 68, wherein the regimen is effective to maintain an immune response to the target cell with fewer adverse effects in the individual as compared to a corresponding method comprising administration of a conventional bispecific T cell engager comprising the corresponding bispecific T cell engager domains of the CID.

70. A method of treating a disease characterized by a target cell in an individual, comprising:
(A) administering to the individual the first and second CID components of the system of any one of claims 28-33; and
(B) administering to the individual the IMiD in a regimen effective to treat the disease.

71. The method of claim 70, wherein the regimen is effective to treat the disease with fewer adverse effects in the individual as compared to a corresponding method comprising administration of a conventional bispecific T cell engager comprising the corresponding bispecific T cell engager domains of the CID.

72. Nucleic acid encoding the first and second CID components of the system of any one of claims 28-33.

73. A method of modulating an immune response mediated by T cells in an individual, comprising:
(A) expressing the first and second CID components of the system of any one of claims 34-38 in the T cells; and
(B) administering to the individual the IMiD in a regimen effective to modulate an immune response mediated by the T cells.

74. The method of claim 73, wherein the regimen is effective to maintain an immune response mediated by the T cells with fewer adverse effects in the individual as compared to a corresponding method comprising expression of a monomeric signaling molecule comprising the corresponding signaling domains of the CID in the T cells.

75. A method of treating a disease characterized by a target cell in an individual, comprising:
(A) expressing the first and second CID components of the system of any one of claims 34-38 in T cells in the individual capable of recognizing and killing the target cell; and
(B) administering to the individual the IMiD in a regimen effective to treat the disease.

76. The method of claim 75, wherein the regimen is effective to treat the disease with fewer adverse effects in the individual as compared to a corresponding method comprising expression of a monomeric signaling molecule comprising the corresponding signaling domains of the CID in the T cells.

77. The method of any one of claims 73-76, wherein the T cells are CART cells.

78. Nucleic acid encoding the first and second CID components of the system of any one of claims 34-38.

79. A T cell comprising the first and second CID components of the system of any one of claims 34-38.

80. The T cell of claim 79, wherein the T cell is a CART cell.

81. A method of modulating the level of a POI in a cell, comprising expressing the second CID component of the system of any one of claims 39-45 in the cell and modifying the amount of the IMiD in the cell to modulate the level of the POI.

82. A method of modulating an immune response to a target cell in an individual, comprising:
(A) administering to the individual modified T cells expressing the second CID component of the system of claim 42, wherein the POI is a CAR capable of activating the T cells upon binding a target antigen, and the target antigen is expressed on the surface of the target cell; and
(B) administering to the individual the IMiD in a regimen effective to modulate an immune response to the target cell.

83. A method of modulating an immune response to a target cell in an individual, comprising:
(A) administering to the individual modified T cells expressing the second CID component of the system of claim 42, wherein the POI is IL-2, IL-12, or IL-15, the T cells are CAR T cells expressing a CAR capable of activating the T cells upon binding a target antigen, and the target antigen is expressed on the surface of the target cell; and
(B) administering to the individual the IMiD in a regimen effective to modulate an immune response to the target cell.

84. A method of modulating an immune response to a target cell in an individual, comprising:
(A) administering to the individual modified T cells expressing the second CID component of the system of claim 44, wherein the POI is a CAR expressed in the T cells capable of activating the T cells upon binding a target antigen, and the target antigen is expressed on the surface of the target cell; and
(B) administering to the individual the IMiD in a regimen effective to modulate an immune response to the target cell.

85. A method of modulating an immune response to a target cell in an individual, comprising:
(A) administering to the individual modified T cells expressing the second CID component of the system of claim 44, wherein the POI is IL-2, IL-12, or IL-15, the T cells are CAR T cells expressing a CAR capable of activating the T cells upon binding a target antigen, and the target antigen is expressed on the surface of the target cell; and
(B) administering to the individual the IMiD in a regimen effective to modulate an immune response to the target cell.

86. A method of treating a disease characterized by a target cell in an individual, comprising:
(A) administering to the individual modified T cells expressing the second CID component of the system of claim 42, wherein the POI is a CAR capable of activating the T cells upon binding a target antigen, and the target antigen is expressed on the surface of the target cell; and
(B) administering to the individual the IMiD in a regimen effective to treat the disease.

87. A method of treating a disease characterized by a target cell in an individual, comprising:
(A) administering to the individual modified T cells expressing the second CID component of the system of claim 42, wherein the POI is IL-2, IL-12, or IL-15, the T cells are CAR T cells expressing a CAR capable of activating the T cells upon binding a target antigen, and the target antigen is expressed on the surface of the target cell; and
(B) administering to the individual the IMiD in a regimen effective to treat the disease.

88. A method of treating a disease characterized by a target cell in an individual, comprising:
(A) administering to the individual modified T cells expressing the second CID component of the system of claim 44, wherein the POI is a CAR expressed in the T cells capable of activating the T cells upon binding a target antigen, and the target antigen is expressed on the surface of the target cell; and
(B) administering to the individual the IMiD in a regimen effective to treat the disease.

89. A method of treating a disease characterized by a target cell in an individual, comprising:
(A) administering to the individual modified T cells expressing the second CID component of the system of claim 44, wherein the POI is IL-2, IL-12, or IL-15, the T cells are CAR T cells expressing a CAR capable of activating the T cells upon binding a target antigen, and the target antigen is expressed on the surface of the target cell; and
(B) administering to the individual the IMiD in a regimen effective to treat the disease.

90. A method of treating a disease in an individual, comprising:
(A) expressing the second CID component of the system of claim 44 in target cells in an individual, wherein a level of the POI in the target cells above a certain threshold is associated with the disease; and
(B) administering to the individual the IMiD in a regimen effective to treat the disease.

91. Nucleic acid encoding the second CID component of the system of any one of claims 39-45.

92. A cell comprising the first and second CID components of the system of any one of claims 39-45.


SEQUENCE LISTING (25 sequences)

SEQ ID NO: 1 - protein, 6 aa, Artificial sequence
  Synthetic polypeptide
  misc_feature: Fab-TC1 HC-CDR1 (cereblon + thalidomide)
ISSYSI

SEQ ID NO: 2 - protein, 6 aa, Artificial sequence
  Synthetic polypeptide
  misc_feature: Fab-TC2 HC-CDR1 (cereblon + thalidomide)
VSSSSI

SEQ ID NO: 3 - protein, 6 aa, Artificial Sequence
  Synthetic polypeptide
  misc_feature: Fab-LC1 HC-CDR1 (cereblon + lenalidomide)
FSSSSI

SEQ ID NO: 4 - protein, 6 aa, Artificial sequence
  Synthetic polypeptide
  misc_feature: Fab-PC1 HC-CDR1 (cereblon + pomalidomide)
VSSYSI

SEQ ID NO: 5 - protein, 6 aa, Artificial sequence
  Synthetic polypeptide
  misc_feature: Fab-PC2 HC-CDR1 (cereblon + pomalidomide)
VSYSSI

SEQ ID NO: 6 - protein, 10 aa, Artificial sequence
  Synthetic polypeptide
  misc_feature: Fab-TC1 HC-CDR2 (cereblon + thalidomide)
SISPSYGYTS

SEQ ID NO: 7 - protein, 10 aa, Artificial sequence
  Synthetic polypeptide
  misc_feature: Fab-TC2 HC-CDR2 (cereblon + thalidomide)
SIYSYYGSTY

SEQ ID NO: 8 - protein, 10 aa, Artificial sequence
  Synthetic polypeptide
  misc_feature: Fab-LC1 HC-CDR2 (cereblon + lenalidomide)
YISSSSGSTS

SEQ ID NO: 9 - protein, 10 aa, Artificial sequence
  Synthetic polypeptide
  misc_feature: Fab-PC1 HC-CDR2 (cereblon + pomalidomide)
SISSYSGSTS

SEQ ID NO: 10 - protein, 10 aa, Artificial sequence
  Synthetic polypeptide
  misc_feature: Fab-PC2 HC-CDR2 (cereblon + pomalidomide)
SIYSYSGSTS

SEQ ID NO: 11 - protein, 16 aa, Artificial sequence
  Synthetic polypeptide
  misc_feature: Fab-TC1 HC-CDR3 (cereblon + thalidomide)
SYYWQYYYQFGYPFGF

SEQ ID NO: 12 - protein, 12 aa, Artificial sequence
  Synthetic polypeptide
  misc_feature: Fab-TC2 HC-CDR3 (cereblon + thalidomide)
TDSWYYYRYGGM

SEQ ID NO: 13 - protein, 18 aa, Artificial sequence
  Synthetic polypeptide
  misc_feature: Fab-LC1 HC-CDR3 (cereblon + lenalidomide)
SEYPYSYWYMYGYPVGGF

SEQ ID NO: 14 - protein, 16 aa, Artificial sequence
  Synthetic polypeptide
  misc_feature: Fab-PC1 HC-CDR3 (cereblon + pomalidomide)
SYFVEYYYYYGWPWGL

SEQ ID NO: 15 - protein, 17 aa, Artificial sequence
  Synthetic polypeptide
  misc_feature: Fab-PC2 HC-CDR3 (cereblon + pomalidomide)
SSYSYWYYIMYGYWFAM

SEQ ID NO: 16 - protein, 8 aa, Artificial sequence
  Synthetic polypeptide
  misc_feature: Fab-TC1 LC-CDR3 (cereblon + thalidomide)
GSQSQMPF

SEQ ID NO: 17 - protein, 8 aa, Artificial sequence
  Synthetic polypeptide
  misc_feature: Fab-TC2 LC-CDR3 (cereblon + thalidomide)
YSWWWMPI

SEQ ID NO: 18 - protein, 6 aa, Artificial sequence
  Synthetic polypeptide
  misc_feature: Fab-LC1 LC-CDR3 (cereblon + lenalidomide)
WGYYLI

SEQ ID NO: 19 - protein, 7 aa, Artificial sequence
  Synthetic polypeptide
  misc_feature: Fab-PC1 LC-CDR3 (cereblon + pomalidomide)
SSYSYLF

SEQ ID NO: 20 - protein, 6 aa, Artificial sequence
  Synthetic polypeptide
  misc_feature: Fab-PC2 LC-CDR3 (cereblon + pomalidomide)
SDSMPV

SEQ ID NO: 21 - protein, 11 aa, Artificial sequence
  Synthetic polypeptide
  misc_feature: LC-CDR1
RASQSVSSAVA

SEQ ID NO: 22 - protein, 7 aa, Artificial sequence
  Synthetic polypeptide
  misc_feature: LC-CDR2
SASSLYS

SEQ ID NO: 23 - protein, 246 aa, Artificial sequence
  Synthetic polypeptide
  misc_feature: Heavy Chain Scaffold
EISEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSHHHHHH

SEQ ID NO: 24 - protein, 226 aa, Artificial sequence
  Synthetic polypeptide
  misc_feature: Light Chain Scaffold
SDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSDYKDDDDK

SEQ ID NO: 25 - protein, 109 aa, Artificial sequence
  Synthetic polypeptide
  misc_feature: IMiD-binding domain of cereblon
TSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIP